SEQUENCE LISTING (49 sequences)

SEQ ID NO: 1 - DNA, 52 bp, Homo sapiens
  source 1..52: mol_type = genomic DNA
aagtgctgcccggcgctccggaagcgattcatcgggccgcgagcgccctccc

SEQ ID NO: 2 - DNA, 119 bp, Homo sapiens
  source 1..119: mol_type = genomic DNA
cagcctctcggccccgcctcgatttttagctttataggaatgctgttgctttaaatccgaaatcccgtgccggtatcaactctcgcgatctccgaggccgcatacatattacccacaat

SEQ ID NO: 3 - DNA, 125 bp, Homo sapiens
  source 1..125: mol_type = genomic DNA
tggcgcgctgcgatgacgtcggcggcacgcctgcgactcgggctccgcgcaaaagatggggttggggtacggcgcgtagagatgacgtcgggttctacgcgcagtggtgacgtcacgggagcgcc

SEQ ID NO: 4 - DNA, 141 bp, Homo sapiens
  source 1..141: mol_type = genomic DNA
cttgccagccccggacttctgcgcgcgctgcatgcccattggatgtgctcattcccaccccagccaatcccgaaactcgctcggacgctgacagaagactgcgccgctgctttgggattggtagctgagttttgtgtcgcg

SEQ ID NO: 5 - DNA, 195 bp, Homo sapiens
  source 1..195: mol_type = genomic DNA
agaaggtggaaggagaaaagaaaacttcgcgccaatcggcgtggccccgcccccagaccgtcccttcgttgcgacaccaactagctcgccccgcccactccgtacacctaagaggggcgcatgacgtacatccgctaacatgcagccgccatcattgattcgataggcgacgcggggagcggaagccctttacac

SEQ ID NO: 6 - DNA, 239 bp, Homo sapiens
  source 1..239: mol_type = genomic DNA
agctcccactgccgctcagcgccgatgcgccgcccgcctcgagctcacattggtcctggcagccttcccgccacaccaaccaaccaatagacagggcgattctgcgctcccggccctgctgcaggctgtctcgcacttgtcattggtcactgcagccgccccaccccccccggcgcgccagtggctgggcggcctcgctggggcgggccgcagttcctgcgcgtgcgcgcttggcctcc

SEQ ID NO: 7 - DNA, 341 bp, Homo sapiens
  source 1..341: mol_type = genomic DNA
tcctgaaagacacgtcagccggaagcaagacacgggcacgctaggaaatgtagtttactttttccgagcggcggcgaagtaagccaatgggaagctccacaataatatgcaagggggcggaaggcgtaagtgcgtcacggagagcatctcgggaattgtagtgtgtttctgcaagccaatgggatccggggaaacatgataaggatgcggtcgaggctgggaataggctgagggagctggagagggtgggaccacgcaggaggcagatccaataaacaggaagatttttctcgtgacgtcgtcggcgcgcgccggaagcgcggatcacacgggcccctaca

SEQ ID NO: 8 - DNA, 343 bp, Homo sapiens
  source 1..343: mol_type = genomic DNA
gtatgcagctaactacttccgcttccgcttgcgtcccctgcgtccctggctcgaggttttccgccattttgagtgtggccctagattgttctcaatactctgtccttgggttttgtaagttgaaagttgagactaggaaggcatatcacaagaactataaacagagtttcaatgaggcaacactgaggaggagttctactccttcctcatccccttccaaagcccctgaactcgagtagccgcccctcccatcatggttcccttagtgtggtctcgccgcacaccccgcccattgacccggaattcttctctgcctggtcttcaggtcccaatcctccgcttc

SEQ ID NO: 9 - DNA, 371 bp, Homo sapiens
  source 1..371: mol_type = genomic DNA
tccggaaatacgtaaaaacagagcgccgggtccacgccttccccaaggaatggggcgtgtctcggcacccctcggtgacgcctccactgacatcatctgcggattcccgccccgaggtttctaaatccagacattcccgtttggctgagcactctaggcctacatccatgaagctaggagagcgacactcaaagactgcactattgagagaagctaacgttaaaggcagtgaatatattcgggagtccagctccggaacccgggagctcttttagtgggaggggcggcgctgatggcgcttctggcctccgaatgctagggggcgctgtgatcgccgggcggcctcttgggcgctgggagtccaccgcgca

SEQ ID NO: 10 - DNA, 441 bp, Homo sapiens
  source 1..441: mol_type = genomic DNA
cgttagaattcaaaggttcttctttttaaataggagttaaaccctctagctagaaagccgattggcctgcgctcgagcgaggctgccttgtgattggttgacaacaaacccgacgccttgagtcgcgtttaagggcgcacaccgcgcgcaggcgcacttgctccctaagaacccggaagtggtggagccaacggccagccagacctgtggcagtcccagccccagggtgctctccgcggtcggccatttatcggtgaagcaacagccccttgacgtcaagcttgacgcattggagataaagtccaaggaggtgaactaacctttgaacgtgccagtaagtaagatggcgagaaaagcaagagagagtgggaccgatcgatagcgcagcggtcggcttggcgccctttcagcgtgcgcagtgaacgtgcgctcggagcggta

SEQ ID NO: 11 - DNA, 527 bp, Homo sapiens
  source 1..527: mol_type = genomic DNA
gctcccctcactccagttgctcagcccgccgctgagcacagccaatccgcggccgtccactcactgtttcggccaatcaccgagcagacggcccggaagctcgagaggctcgtgccaggtcggggggcgaaggcgaggccagcccaccaagcaggggcgtcaccaagggaaagccaatcaccattatttctgaagcccaagggggcgggacgagaagtcaacataatggtggtgctgctggaaatctagcacctggggagagcctgagacttgtgtatctaattgcctattggacacctccatctgggtaaacccaggcatccagaactttccctcaagacttacctccctcagtgcaggtcgggctggcctcacgcatttcgcttggatgcctaaatctgtctccacgtggggccggtcgataaaacaacacttcccagaatacgctgcgatccattcgcggtctgcacccggaaagtacacctcccaggggtctgtgcgcgcctccagggaacgggtcttgtggc

SEQ ID NO: 12 - DNA, 546 bp, Homo sapiens
  source 1..546: mol_type = genomic DNA
gtcagctggttgtgtcacttgacccctcttgttgctaagaggcagcagggacacccattttcacttccgcttccggttgtcacaggcgacgaagggggaggacagaacagaggcgttgcccggatgtcgaggcgcgcgcaccgcggaaccacccctcaattccggttcgcctcccaacccctccccatccagcttccctgcggctgcgcaatacgcggcacgcgccaggccaaagactaggagcgaagcgtgcacgccttgtctttctccacctttcgtctttggtcacagcgccatctcatggttagctgaaactttgaaagaggattcattcttcagaggcaaggaatcggagtcctatgtctttctcgcgccttgaaggttacttagctaaagctaagatagcgagaaacagtacttttcaggtccttccatgatctacattcgtccttgttctcggaggacccagactctatggttaatgatgaagaggggcctctattcgttcagcgcttgcgcatgcgaggaggttccgcatgcgcggtg

SEQ ID NO: 13 - DNA, 611 bp, Homo sapiens
  source 1..611: mol_type = genomic DNA
ccgcgcttgctccgccgcgctacaggtcgcgggaggggccaggcctaggtgcttcgccaaaagagggcggagctgcgcacacgtgctggctccccgccgcctcgggctcggctctctccgcgcagaggtttcatggcttgcttcaatcttttgatatcttagagtaattgcttccaatttttgggcatcgccgcctcagtttacagaatagtatggatctggtgccagagccgggaaaacgatgcgcatcgtaaaccgcaaaacactgagcatccaggacagaagccaaggcgcttggcttggttgaaggccagtgcgcatgcgcgagtttacccttcgcggactgggcgagctcttgccactcctacctccgggcttcagtcttcgccgcctgttctggtcctttcccgggccgctattggaagaaccgtgaactatggaaggcccctctggattggctgggctggcccaagagtaaggagagccctcttcctggcggtggggaagggacggcggcgattggcggacgctcctggcaggagagcgcggattggtcaggcacggagcaggaggcggggctgatagcccagcagcagcagcggcggcggcgg

SEQ ID NO: 14 - DNA, 736 bp, Homo sapiens
  source 1..736: mol_type = genomic DNA
agccggtacgccccagaggctcaccggaagtgccggacccgagacccggagggggggtgcccgggacaaagcgtcggctgcaaaagagaaaaggccaggcgaagacgctaggctctaaaacaccctcagtagaagcattaaaatgccctttcgtgatttatttccccttttataggtatttttaaacgttattttaaattacgtgttgacagtattcccctccagacgaatttagtagggcccattgtccttacgactacactacccacaatgcactacccagactgcctctgcttcagccatcgaggactcgggcggaactaagctacaaatccttcttcaaaccacaccaaaaacctattgcaaacgtaaatgctgccccattgggtatgtgactgagtttaatgtatggttagaagtatactttccctgagggttcccagtgggaggcagtaggcttctcttcccaaagtaaagatgtccgcccagcgtcgcttttctgggtaaaagcgagtcagagttgaagggggcaacagtgtttgcgttcggcctcagaggtgacgcttctttgggggcgggctggagttttctggccgtgaatggcagagcgctaatggccgcctccaatgtggcccaatcagaaagaaaggaaggctgggaactaagaagctattggttggtgatccctggaccaatcggaggagccgtgatttggcgggagtcttgaccgccgccg

SEQ ID NO: 15 - DNA, 737 bp, Homo sapiens
  source 1..737: mol_type = genomic DNA
ttcttccttgtcaccctccgcagtttggcaggacagccaataggcacacgagatcctccatcaaggggcgttcccatctggggatccccacgccggcagcaccaaggcctctgggtcagcttctttaagggcaagcgcccttagagaagcagcacggaagcaaccaatcaactggtttctccaattagcccccaagtggatcagcctgctgaactgtcggctagggttataggtggttctgagtccttccggaagctgccgaatcttagaagtcgcgccactgttgtggcgatggaagaaggcgaagctctacagaaaccattttggttgagggaagagtggttttaaagttaatttggaaaggagccggcaagcaaccttcagtcggtgtgcggtttttacagttgaactgtttccagccgctaattatgattgttttagcggttcgtcatctgaaaactgcattctgcacggcggtggtggggggaggacgaggatggagaagcctgggcggacatatttattagctgcggtcctaagtcacgtgtgacattctgtatccagtctatcggttatcccaaggagaacgccctgccggaatggccaatcgccttgagatgtccccgccctattcgccatcttaacatagggcattccccacagctgcaggcaccagtgaagcgtctaagggaccatcttggttgagggcggaagtgaggactgtgtcgcgctggagg

SEQ ID NO: 16 - DNA, 813 bp, Homo sapiens
  source 1..813: mol_type = genomic DNA
gctgtgacctttcaccccgcccctcgcgcgccggcgcgccgccgcctcacgcgcagccgcactgctcgcctccgcgcaccctgccccctccactccggaacctccatctttctcctggactgggccttctggggtcatgctcctccctcccacccgcttacgacactgcttgtggggctggctcggtaggccctggcggtaaaatccagatttttccacccctccctctcttgtcccatatccgcaagatagtttttctcagccccgttcttcccagtttgtggtctccagagtgaccttcaaacgctgttgcctcctagtagggttttataaagttagttcttctaaatgtaaagaaatctgtggaaagctcaagtttaagttaggtgttttaaaacaaacgtagttaacaatcggccagatagattggctaccctccagcacgcctcaagttttaagatcacgtgattggcccaaacctcagagcctggaattttaccggcggactccagagggacggaacgccaccgccatttcctgtccctcgattgtggcgtgaacgtcacttccttagcaatgcgctttaactcttgctgtccacattttccacattgtactttataacatactctttgtgaaaatttatcttttatttctttaatccaagaaaattaatattcaagggatccgtaaaaattcaggccccccgagatgtagagcgctgggatggaggaggcagggcggagctcgacgtcatgtgactggttgggcccgcccccccccccccccccaacctgtgcagctgaggacttc

SEQ ID NO: 17 - DNA, 1433 bp, Homo sapiens
  source 1..1433: mol_type = genomic DNA
actctcgggtcttcccgcggccgccgaagacccgctgaggcagaagctgcccgcggcgcgcgccttatcaaaaacccgttttcttgccattgaaagagaaactgggaacttagataacaggaaaatctccacatacactaaattcacattattttataccctgtaaaagctcccaccagatataaaaactcattataatggcatgcgttccgtcacaacttcggacagaagttgggtctttgggcaggaagtggaagccatagaggaagtcatggagcgagagtgatgtctgtagtgaagaatttggcgccatcttgagaaggtcaaagtgattcttcgattaaatgccttacttatcgcctagtgtgacctggcgcttgggtttactctccccagcaactgcgcctcaaacgcaaagaaaactgcaggtcctcggggaaggatcgatttcaccattcaagagaaaaatccatttcccagattcctcccctttatgtaaacgtctggtacctagtagacgttaaatacacgtgagttttccggttttataagatcacatactgcaaaccaacagtaaattacattaatacgcatttctttagaaattcttaattgctaggcactgacatataaattgagtttgctgttgttttagagagaaaaatagacatattcattttttttcaatcaggacatggtattcgtttccttcttaagcttcccatttttgaggtaggaaagtattggtgtgtgtctctatctctaccttgctccctcattagcaggcttaatgggcaggaattatgtctgttttgctttccgttggatccccgggtctagtagagcgccttgacacatagtaggtactcgacacagatgctggcttcatgatgaagcaattgtcctcgaattaatacaaaagggaagattcagactcagcgagccggtaccattcgtctagggacacacagccaggaaggaaccgaaccaggattctaacccaggattacccaagttcgaattctaattgaacataagtccttagcgaccttggacggacttactaaactgccctgaatctcggcttccttagctgcaaaatagggttaggaaaagtggactcggtgctcctctgcgccgcattcccgcagcgggatttgtgaaagcgaacttgagccccacactgcagaagccctgaggtcctgtctcaaccagaatccagcacccaggacaagccctccagcgacttcagaccttcccgctcccgaccccgcccgcccccgagaggcgtggtcaaggcggggccagaaggcggacgcgtgcacggagcggtaaagcgcaggcgcaaagcttcggtgacgtcagagaggcgcgctcccagctcggagccgactcgcagacgcgccccgcccctcggcgtcgctctggactgg

SEQ ID NO: 18 - DNA, 2020 bp, Homo sapiens
  source 1..2020: mol_type = genomic DNA
gtctctctgcgcatgctcacacgcgacctccccacaaagctactccgcgggcctgcctggctactgagtcgcgaaaatatatcgtagtgccgcaatgacttctgggtactgtagtccaacatgtggactggccactcggcttgaagcttccttgggattggatgttgggaccgtccaaacacggcgacttctcatttgtcagcctgcggcagtttaaattgggtggcggtggtggcatgtcctgttagctgtggagtcttttcctcttatcttgcgcctccggttgtgccttaatgacacttttgacattgtgtatcacaaccaactggcaaaaactagtaaatcacgggctcctgacgtggcagctctggctaaaatgcaggtggacgtcctaggtgcccagggagttcgggctcactggggagtagggtgaaggaaaccctgaaaccgagaggctggaagcccgacttgcgcagcataaaaacacctgcagggggaaagcctacatgaggtacgggttgtcatcccgtctcatcttggccatctgactgatggagatcctcaggcacccagcagactggggtgtcacccccactacccactgcccagggctgacctcgggcggaagagcgacattcaaatatttgttgcatatggagcaacagaccagggtggaagagagagaaaggactttccaggaaggtccaggcttgccaggacttctatgcttggactgctctgtccccaagctcgctggtgcccatcaggagcctgcttggcacctttgcttatttcttcgattgttcctcaactcagagggggagaggcaagattatcactcccatagcctataacatgcacgcttaaacacaacccctctgacatggcttcagaccgcatccacaggtgaaaagcaccaaatccaaacaatcaggccacaaacccgtcaagagctcgccggtctggccgtccagtcgccgcccgccttgagactcagcctgagctacagcttcgaaactagcctctagccttctgggtctcagcacaactcttgagcaatccaaccctgtggccgtgggcagataccaagatgcacagttagcacctccgacaggaccgcggctcggcggggctcgccgtctacacgcccattcccaggcccacggccacgacacccagagctgcctcctgccctgggctccctggagcctggtggccgcggctcatgctgcagtcaccgtgtggccagctgccaagcgccactacttgcacctgaagtcgcagccccgcgctgcgcacccagtcgaggcgccaacacgcgaactgggcgggagtggccggcccgcgggggagcgaggaacgacagggttccccccactcgctgcccagactggccgcgggatagcgcgccccccccaaagcctgcacccctgccctgctcctctatcattccgtcccccttgctctgtcaaaccctgccggggctgctctgtctctgccgacccgccacttgcccgaagttcggcctgaggagcttggggccctgaggagcgccgacgtaggcagccagccagtcaatcagtcagagggtcacggcggcccctcggctactcagtggcagccaggccagaaagaagcagccgccaaccctggagcagaatcctacagccgacctgacctagtaggcccccagccctccagagcttggggtattgcagagccgccacaaggcctgccgggagtagtagtttgtatagttcgcagccggccagggcggagccgggtgggttggtggggagggcggggaaacaagcctggaccacaactcccaggagtacccgcgacggcggcggcctcgctgtcgcactcaggctatagggggcgcattgggcggaagaccctgggccggcaggaacttgcttgtgattggatgttgtgggacggcggcttctcattcgtcagcctgtgactgtggagtttgaattgggtggcggttgactgtagagccgctctctctcac

SEQ ID NO: 19 - DNA, 4357 bp, Homo sapiens
  source 1..4357: mol_type = genomic DNA
cacatctgcggcatcctctcggccaccgtagagtgtcccacgtgtccgcttcctcttccgctactcgcccgcgcgtctgtctgtcaagagagacttccggcatgccttttttttttttttttttccctttccccgcaccgtcacctggtggcgcttactggaagccgcaccgaggcgtgtgctgtattttaaaaatcccatttctggtcaatttctccacgtaatcagacacccgcagagaaactgcatataactctttgcctcaaggcttttagacaagcagagggccaaaacttgcataaaatggaaaatcatcatcattaacatcagtaccatctaaattaagagcttctaattgttttaagggaaggtggaagctaaaatgaaccggcccatccaacagagggcactatctgtggaggaccaacaaaggaaaacagagcggaaaatatgggcgcacatgtgcctccagttcatgaagttatttggagggaggggggaagaattaaacttacattattgattacaaaaacagttgaaaatctgttagagtgaaaacagccatggtaatctacagaagaaaagggagaatggaaagtaaagatcattcatgtcttgaaataagttgtctacagtctgtgagaacttgtgggaggtgatcctagtgtgatatattagtgcatgagcctagcaaggaaatcacatcaccacagggcttagcgcttagaattagaactaagtcctcattatttgtgagaaactcaaaacagaacttaacaaaagtgtttaacaactttcaatttttaagaagggacgctatctcattttcagacgccatggatcctcactagcaaaacacaatgtcacagacaaacgaggcatatcaggtttttctaactcatttcggtactccatcacttaatcatgaaataatgttgactatatatatatatatatatatatatatatagtcaacattatgggttttttttgttttttttttttttttttttttgagacggagtctcgctctgtcgcctagactggagtgcagtggcgaaatctcggctcaccgcaacctccgcctcccgggttcaagcgattctcctggctcagcctcccgagtagctgggactacaagcgcccgccaccacgcccagctaatttttgtatttttagtagagacggggttttactatgttggccaggctggtctcgaatccctgacctcgtgatccgcccgcctcagtctcccaaagtgataggattacaggtgtgagccaccacacccggctctgatgtttacaatattataccaacaagggttggatcgtgtgtgacttatctagaggttaagagcaaaagttatgaaatcacagtgccatcatgggagtctagctttcaccatttaaagttgtgtaggcagccaggcgtggtggctcacgcctgtaatcctagcactttgggaggccaaggtgggcggatcacctgaggtcaggagttcgggaccagcctggtcaacatggtgaaaccccatctctactaaaaatacaaaaattagctggggttggtggtgtgcgcctgtaatcccaggtactcgggaggctgaggcaggagaatcgcttgaagctgggaggtggaggttgcagtgagccgagaccatgccattgcactgcagcctgggcgacaagagtgaaactccatctcaaaaataaaaaaaaataaaaaataaagttgtgcaggccgggcgcggtggctcacgcctgtaatctcagcactttgggaggccgaggtgggcagatcatgaggtcaggaatttgagaccagcctgaccaatatggtgaaactccgtctctactaaaaaatacaaaaattagcctggtgtggtggcgcattcctgtaattccagcaactcaggaggctaaggcaggagaatcgcttaaatccgggaggcggaggttgcagtgagccaagattgtgccactgcactccagcctgggcaacagagcaagactctgcctcaaaaaaaaaaaaaaaaaaaatatatatatatatatatatagatatagatatagatatatgtgtatatatacacatacgtgtatatatacacgtatataaagtgtgaacttgggaaagttatttaatcttattgtgtgttagtttccccatttataacatgggtgagtaatatgtatcctatttcttagagtcaatgggaaggttaaatagcaaaatgaaggtaaaagtaattagaatgtaacaggcacattacaatcactcaataaatattcttctgcctcagcctcccgagtagctgggactacaggtgcgcaccaccatgcctggctaatttttgtatttttagtagagacggggtttcaccatattggctaggctggtcttgaactcctgacctcgtgatctgcctgcctcggccttccagagtgctgggattacaggcatgagccaccgtgcccagccggctgactattttcttaagaataaggatagttggtcacctcgggaggctgaggcagaagaatcgcttgaacttgggaggcggatgttgcagtgacccgagatcccgccactgcactccagcctgggtgacagagcgagatgcggtctcaaaaaaaaaaaaaaaaacaaaaaacgaaaaaaaagaaaaatagtcggccggggacggtggctcacgcctataatcccagcactttgagaggccgaggcgggggcggatcacttgcggtcaggagttggagaccagcctggccaacatggtgaaaccccgtctctcctaaaaatacaaaaattagctgggcattgtggcgcaggcctgtaattttagttactctggaagctgagccaggagaatcgcttgaatccgggaggtcgaggttgcagtgagccgcgatgaagccattgctctccaacctgggtgacaagagctagactctgtctcaggaaaaaaaaaaaaagaaaataggaaagctgcccgcaaagcctgcaccttggacacctcgtctgcactaacatttgggaaattcttcttattatttctccggaacatatatatttacattctttacatgtctgtttcacgttaaagaaaatatttctggagcaatcaatttttttttcctttttttttttttttttctgagacggagtttcgctcttattgcccaggcggagtgcaatggtgcgatctcggcccactgcaacctccgcctcccgggttcaagcgattctcctgtctcagcctcccagatagctgggattagaggcatgcaccaccacgcccggccaatttttttttttttgtagtagagacggggtttcgccattttgcccaggctggtatcgaacttctgacctcaggtcatctacctgcttcggcctcccaaagtgttgggattataggcgtgagccactgcccctggcttccaatttttttttccttttttttcttttcttttttttttttttctgagacggattttcgctcctgttgcccaggctggagtgcaatggtgtgatctcggcccactgcaacctttgcttcccgggttctggcgattctcctgcctcagcctcccgagtagctgggattacaggcatgagccaccacgccccgctaattttgtattttgtatttatttatttatttattttcgtagatacgtggtttcgccatgttggccaggctggtatcgaactcctgacctcaggtgatccgcccgccttgccctcccaaagtgctgggattacaggcgtggccatcgcgcccggccaaatatttttcaaaatctaattttgcataatgaccaagatttgcagaaaaatacaaattaaagagatcttttaaagtaaagattcgtcataaaagtcacagaaataatactcatgaataataaattagaatgggcaatgttaagtacttgaaataacaaaggttttagagtcagggtttctgcagtttccctagccatataacgaggagactgttagagaggatatcctaaaatgctttccaagtttacccacctctaactcggaagaaggcaatttaaactaatttggtttaccggaattcaaatacgtacaattcaacataatgaattgaactgaaattgaacacctgcttgcgtaaacctagtcggctcgagttgttctggttctggattggtcagtctagagttccaggggtgtggccaaaagtctgtgaggcggggccgtaggcagcctaaatcaggcatcggcgcggtcagcctcgtggcgcgcccacgcccccacgccggctcttcccggggtccttccgtgcgcgttgatatgattggccggcgaatcgtggttc

SEQ ID NO: 20 - DNA, 9648 bp, Homo sapiens
  source 1..9648: mol_type = genomic DNA
aaaggaaagctaggggccaccctggccgcttttcagggtctgcgcaggcgccttgagcactgcgtcgcgacgggagagagctttacggcttctgttgcggctaacgggcggggcgtctggtcagcggcgggaaggcccttaggcgctcagagcgggcgggccctgtgaggcggaagtggcgggctctgtgggcggaaggggcggggcctgggagtgtcgtgtgcggactgaagggaaggctgcgggagtgtgcttcgcgtcctctgtagtcgactaccgcagctcttctttgcattctggctctttaataatctcccttcgccctttctttaaatcagttcccaccctcccaacacctgtaatctttctcccctctggactccctcctccccgaatcccccgcatcagaatcttcctttggaatctactgcaagaaatctggctggattctcagattaaggacttccagaatcctatcctccctagaatccccctccttggggatcctctcccagaatccttttcccctcagagccccttcccctcagaatctccctttcaccagaatgtccttcctttcagtttcctcttcccagaatgtttcccatccttctctcagatggccaaccccaaatgtccctcttaagagtgctcaacttagaccccccttttccctgaatctccctccattcggtcctcgtcccctcagtcacccttcccagaatctttctcttgtcaggacccagtacccctggaatctccctcctccagaatctccctttcccggcaaatccccctttcccggcaaatccccctttcccagaacaccccgccccgcaaatccacaaaaacttttctctcagagacccgtccctcaaaatctacctttctcctgagtttatttcccttccgagactcctcttcccagcaactgccttccctggagttttccctccgttagaatccccctctcctaagagaccctcccccaagaattaacctcttccccgtccccaccctagaatctctctcttttttttgagacggagtttcgctcgttgccctggctagagtgcagtggtgcgatctcgtctcactgcaacctccgcctcccgggttcaagagattctcctgccttaggctcccgagtagctggaattacaggcgtctaccaccacgccctggtcgttttttgtatttttagtagaaatggggtttcaccacgttggccaggctggtctcgaactcctgacctcaggtgatgtgatccgcctcagcctcccagagtcctgggattacaggcgtgagccaccacgcccggccgaatctctctgttttttgttttttttttttcttttttttgagacagagtcctgctctgtcgcccaggctggagtgcagtggcatgatctctgctcactgcaacctctgcctccccggttaaagcgattctcctgcctcagcctcttgagtagctgagattacaggcatgcaccaccacgcccggctaatttttgtatttgtagtagagacggggtttcaccatattggccagactggtctcgaactcctgacatcaaatgatccactcatctcagcctcccaaagtgctgggattacaggcgtgagccaccgcgcccagcccctagaatctctttctccagaatctcccagtcatctttttcctctcccccaaatcaccctccacttaagcctcctccccacagaattctccatccccagccccccaaccccacctgaggaaccccaggattgggttgtcctctggttccccggcagatgtccctgcgtctcaccccatcaagagtctccccagcgaagaggttcctctctcagagctagagggagcccacagttgcctcagtttctctggcagtacagggtctgggatccagggttcaaaagggcgtcgtggtgcaccttgccaaagagggtggagacagggagacactggagggcaggtctggggccagagggaaaagccagcccggagtccagctctaaaagggtgaggggggagggggctgttgggagggaggaaggaagtacccagaggctgaccgggagggacacagacacagcccaaagaaatgtaggcacaagtggagaagaagagaaagatgaaaggccaagggagaagaggaggagaaaagggagagaccaggacagtcaagagatggacccccaaaacaagcccagtgacagatttagaggcagagaggccaaccctcagacaaaaaagagataaaggcaggcatgatgtttccaaaatacttgagacagaggccaggtgcagtggctcacacctataatcccagcactttgggaggccaaggcagcaggatcactcgagcccagggttcaagaccagcctggccaacatggtgaaaccccgcctttactaaaaatacaaaaatcagctgggcatggtggcaggtgcctgtatcccagccacaagggaggctgaggcaggagaaccgcttgaacccgggaggcggaggttgcagtgagctgagatgtcgccactgcaccctaggctgggcgccagagcaagactctgtctcaaaaataaataaataaataaactctgtctcaaaaataaataaataaaataaaataaatagagcagagacctcaaatattcacttgcagacacagataaaggctagcacaggtgttttccaatgaagacacaggcagatgagaggatgcacacattctacctgtactttttctttttttttttttaaattgagatagagtctgtgtcacccaggctggaatgcagtgatgcaatctgagctcactgcaacctccacctccccggttcaagtgattgtcctgcttcagactctggagtagctgagattacagattctcctgcttcagactcctgagtagcccagatcacgggcctccaccactatgcttgactaataatttttcttttttttttttgagatggagtatcgctctgtcgcccagactggagggcagtggcttgatctcagctcactgcaacgtcagcttcctgggttcaagcaattctcctgcatcagccttccaagtagctgggattaaaggcatgcaccaccatgcccagctaatttttgtatttattagtagagacagggtttcactatgctgggcagactagtcttgaactcctgacctcaagtgatccacttgcctcggcctcccaaagtgctgggattacaagtgtgagccactgtgcctggccactaatttttgtattttttagtagagacagggttccatcgtgttggccaggctggtcttgaactccttacctcaagtgatccgcccacctcggcctcccaaggttctgggattacaggtgtgagccacagcggttggcctagcctgcactgtcttaagggcctgttgggcaggggagttcaggttctctcgtctctccagattgatcttcacacatttgctaagtctcgtatcgtccctgagcttgggagtcagggagcgctctgacctaagccctcctgactttggggtcaccatctttccccagcctctatgggcacactccctgaattatctgtggcttgagtggagcaagcaggggatgggatgaggggtggcgggggagttcccctcatccacttcagccttccagtctgctcccccttcctgtacctctctcttccagaaccccaggccaagcaccagagaaggcctgggagtgagcaaggtgcccatgttgcttctgtggatcatcctcctgcttgtacagccctgggagggcctaggaacgcccgcccctctgctggagggagcctttttacttccttgcagccctctgaagataccagcgagtggggagcagagccagcagacggggtgggccaattgatggggcgtggtagggggtgtgtttgaggaagggtcagcctctttccctccaagatctgattctcctccttacccctgaactgtaggaaaacaaaaatgatgacaccctctacaccccagatgatctctcagtgagtattgcattcgcttctttctgcagttgcaggttttgagagaattcagtggtctccatgtgtgagggctgctgtgtgaccatatgaatatgttgcggggaggaagtagtgggactatctcctgacccagcccctagccttgacagatatattttgctcactgctgtctccaacacctagaatagggaggtatataataacaaatgatgatgatgactacagctgcatgtaaatagtgtttactctgtgccaggtattggtttaaatgctttaagtatgttagcttatttactctgtacaccaatcctataagatagataggaccaccatgtatggatgtgaatgttgtacactgcacaaggatcccacatctacaaagacagtactatattcacaggctggatattgtagatttgcatatttgccaacttgcggcagatggcagtaaagtatcttgttctaataaaatcaagacttagaaggtagaaggaacttaaggaaggtctttttattttcttttttttgagacagggtcttgctctgtcccctacgctggagtgcagtggcacaatcacagctcactgcagtcttgaccttttaggctcaagcaatcctcccacctcagcctccagagtagctgggaccacatacacgtgacaccacaaatagctaatggctaattttttttttttttttttgagacacagtgttgctctgttgccagctggagtgcagtggcgtgatctctgctcactgcaacctctgcgtcctgggttgaagcaattcttctgcctcagcctcccgagtagctgggactacaggcatgtgccaccatgcccagctaatttttgtatttttagtagagacagggtttcaccatgttggccaggatgttctcgatctcttgacctcgtgatccacctgcctcaacctcccaaagtgctgggattacaggcgtgagccaccgtgcccgacctttttttttttttttttttttttgtagatatggggtgtcgctgtgttgcccaggctggttttggacacctgggctcaagtgatcttcctgccttggcctctcaaagagctgggattacaggcatgagccactgtgcccagctgagtgtctttttctagcttgcacataagtacagcatgggatagtgtcaggccttacggttgggattatccagagccctatttacagatgaggaaactgaggcccagagaactgaaggggtttgttccaggacacagataagaaatcttgggagtggggacttggactcatgcccttacccatagataccaggtcactttgtccctactgtgtgtcctgctgagactctaggctgctccaggttggagctgtctgtgattgggtttgaggagggaccatctatgggaactgctgtgttccagctacagtacctgctggatctcctggggtcccggctgtgggtggccagccagggcccttgcctgtcctgtgaaggacatccccagagacctgtccccctcttggagttactacagggagcttgtgcttggcaccctaccacagcctgagctttggaggacatgggctcccaggtacacccagagaggctgggtagagagagggagggaatagtcttcagctccaactgccaaaccctaagccttttcttccctctaggccagaggaccccacctggagccctagtagccaaggggaagagttaagaaggaggaaagtaaggtgcgatctgaagtctctgacctcttgggcagggagttggataaattgccccacctgcccccattctcaccctcctttagatactcttctctcttcttccaggaagggacagagaagtgcacaggtattctctaacctcatccctataccttgagaacagggagggtcctgtggttcacatccctacatccctggctacaaaaaattaccacttgaagaactttcttttcttttcttttctgtttttgaggcagagtctcactctgttgcccaggctagagtgcagtggcatggtctcagctcgctgcaacctccacctcctgggttcaagtaattctcctgcctcagcctcctgagtaactgggattacaggcacgcgccactacacctgactaatttttgtatttttagtagagatggggtttcacaatgttggtcacgctggtctcgaacgcctgaccttgtgatctgcccacctcagcctcccaaactgctgggattacaggcgtgagccaccgtgcccagcccaccacttggagaactttcaagtaatactgatggccagaccccagcccagataatttgataattggtctctggtgaagcctgagcatctatattatttttattattatttagagacagggtttcactatgctgcacaggctgtaacttggcatctgcccatatatttttttgagacagggtctcactctgtcacccaggctggagtgcagtggcatgatcaaaactcattgcaggctcgatctttcaggctcaagtgactctcccacctcagtcccccaagtagctagcgctataggcgcatgccaccacactcagctaatttttgtattttttgtagagatggggttaccctttattgcccaggctaatcttgaactcctggctcaagcgatactccccccttggcctcccaaagtgctgggattataggcatgagccaccacacctgacattttttttttttttttgagacagagtctccctttgtcacccaggctggagtgcaccggctggtggactggatggccacatttccatcaccccaaagcattccttctctctcttcccagtcaatttccacctccctctccagccccagacatccactaatctgcattgtctgtataggttggttttgcttgctctggagtttcatatgaatggaatcatgctggacatctcctgatgtctggcttctttcactcagcataatgttttggagattcagtcatgggtgtattggtagttgttactattctgttgcatggatggatgtaccaaaatttgtttttcttttgagacgagtctcactctgttgcccaggctggagtgcagtggtgcgatctctgctcactgcaaccttcacctccctggttcaagtgattctcctgcctcagcctcccaagtagctgggattacaggcgtgcgccaccacacctggctattttttttttttttttttttgtatttttagtagagatggggtttcaccatattggccaggctggtcttgaactcctgacttcgtgatccgcctgccttggccttccaaagtgctgggattacaggcgtgagtcactgcgcccggccctaatttttgtgtttccagtttagggctattatgcaactctttctttctaaaatattctcaagtattctgatgtgctaccagagttgagttcagaaccacggatccaccaaggaagaaacttccatggctggggaaatgactgtgccctcttttttctgcagggaatggctttctgcagcagtgtctgtagcgatttaatatgtagaatcctattttcttgttgtttttttttttgagacggagtcttgctcttttgtcacagcaacctccacctctcaagttcaagagattctcctgccacagcctcccgagtagctgggattacgggtgtgcgccactacgccacctggctaatttttgtattttttagtagagacagggttttaccatgttggccaggctggtcttgaactcctgacctcaggtgatccgcctgccttggcctcccaaagtgctgggattacaggcgtgagccaccgcgcccggtggagaatcctttctttttttgagatggagtctagctctgttgcccaggctggagtgcagtagcacgatctcggctcactgcaagctcggcctcccgggttcacgccattctcctgcctcagccttacgtgtagctgggactacaggcgcccgccaccacgcccagctaatttttttttgtatttttagtagagatggggtttcaccgtgttagccaggatggtctcggtctcctgacctcgtgatccacccgcctcggcctcccaaagtgctgggattacagacgtgagccaccgcgcccggcccaggtgtgtcatacttatgtgagaaattttaccaatgtgtcctgaggcttcttttttttttttttttttttgagactgagtctcactctcgcccaggctggagtgcagtggcgcgatctcggctcactgcaagctccgcctcctgggttcacgccattctcctgcctcagcctcccgagtagctagaatcacaggcacatgccaccacgcccggctaatttttttgtatttttagtagagacggggtttcaccatgttggccagactggtttcgaactcctgacctcaggcaatctgcccgccccggcctcccaatgtgttgggattacaggcatgagccaccgcgactgacatagacttctgatttttaaaatttcccctatcaggttaatttttcaatttttaaaaatttaagttttaatttttttttttttttttttttagagacagggtctctattacccagtctggagtgcagtggtgcaatcatagctcactgaagcctcaacatgggttcaagtgatcctcccaccttagcctttggagtagcagggaccacaggcacacgccaccacaccacattaaaaaaaacacaaaaattggatacatggggtctcaatttgttgcccatgctagtctcaaatacctggcctcaggtgatcctcccgcctcagcctcccaaagcgctgggattacaggcgaaggttcatttttcatctctccattacacctgtccactctgccatcactcgtcagccgtcattactaatagataaattcgcctttaaatgaacttaggaagcctggaaagaaaaatagggacttgggccgggcgcggcaggttatgcctgtgatctcagcactttggcaggacgaggccggaggatggctttttagtttgagttagaggagtttgagaccaggccgggcaacagcccgactctaccaaaaaaaaaaaaaaagtacaaaaagaaagaaaaagaaggaaaaaccagggcttcacggtcactcagacttggcgaggaggcttgggtctgccgctttccactgagtttttctgagcttcagcgttctctccaaaacggggatacggatctcctggggatgctttagagggcctaggagataaagccttgtttcttttataatttgctattcgctgatttcgccgcctcctcgcagcttggcctaccgcgctagtgttccggactaacgaacaaacggctcactaaattcaaacatgagtttcagcctggtcgccgagccggcgggtagaaaaaacctttcccctcctaaccctccgcagatgtcggccccgcactccgcccaggccagccctgcctctctcaagatggcgacctggaagtgcgtcgcccgtgccagccccggaaatctcgtgttaggcgaggtcaacgggtaggttctcgcgagaggacccgtcagccccagtcaggcgtc

SEQ ID NO: 21 - DNA, 12561 bp, Homo sapiens
  source 1..12561: mol_type = genomic DNA
gaagaagctggcccacgcatgcgctcttcaaatttttgagacagtttacccagaatgcagtgctcaaaggaaacgcgtgcgcagtgtggtcaggttgtttcgctgggtgagtaaaatgaaatcttagaggcgttgtgggctggcccagttgatgacgtcaccataccacagcttctagtgctattctgcgccggtatccgaccgccgggcgcggtggcgcgtgcctgtagtcccagctactcgggaggctgaggctggaggatcgcttgagtccaggagttctgggctgtagtgcgctatgccgatcgggtgtccgcactaagttcggcatcaatatggtgacctcccgggagcgggggaccaccaggttgcctaaggaggggtgaaccggcccaggtcggaaacggagcaggtcaaaactcccgtgctgatcagtagtgggatcgcgcctgtgaatagccactgcactccagcctgggcaacatagcgagaccccgtctcttttgaacaataaatacgttaattttggactctcaagagatgaatattctcatagtcttttttattgattaaaccttttactttcttttaattaaaacgttacacttgggaaaggagtttttcctcgactcaccagtgacacctactggaataaatttacaaaacaacttaaaatcagctgtaatgatcacgagtcaacggtagggtttgcttttattttgttaaagagagtacgttttcaaatttatttttttgacacactcctccaagaaatacacctacaccattccaaaaaaaaatgagattctttggcatttagtgtatttgattcttaaatgtaattggctgaatgtaaagtaataaacaagtaatgtgcagtgtatgttgctttgaccagataatttggtgacaaaacaagtaatgaaaagcaaatgcaaaaatgcaaaaaaccaatgtcaataaacgaatttctgctgaaagatacctacattccatatggtcatttataatgcctccagataaaactgctctgaaatcaaagctttaactagatttataaaatgtagcacagggtgcaacaattccagaaacttttacatataaacttttataaattgaaatacaacacttctttaaggtatgttatgtcagggttctagtggggtccccctacccaatcattaagttaggcattgtattatatgattaaggaaggaggggagtcagagatttgtaaactagatataacttgctccttgccctcaaaattttctaatctgtgggaaacaggattatctccaagacttcaacatacaacgtttgtgcaccaacaacatatgtacagctaaggaaaatattctgtattaaatcatgggtgtgctcatgaagtacgaaaggagccaagaagagaaattaattccagttcgaggtacatttgagagacctttgaggtagaccttgaagaatgggatttctacaggcagcacttgacagaagggaatgcctggtgaaaggaagcgcttgagccaaggtaaggtgaacaactttgtgtaactagaatagaagatggacaaaaggcctagtgataggaatgaaaaggtattttattttaaagcctcattgggcaagatccaatgtcaagcttttacaactttcctataatttatatcagtcactaatgtgtggtttccaaaagccatttgcctctcaaaagctaatctaacatttgtccacactttcttaggcctcttctagttggtctccacaatatttaaaacattacagtggttctgagatcaattccattaatatttttagtactctgatatataagcctttgggcctaaagaagtgaattcatttaggagagaaaaaaagacaaatgagaagcaattagttgcacttcaacaatcttaatctgaatacttaagtaattcttaatacttattctagtctatacattattctctttgatagagatattgcaaaagtgcagctaagacgtcgtacttcctttcagttttaagataatgtgacaccatctatcccaaaccatgaaattatccatttctttttcaacaaactgagaacacagctttaaaagttcattttaactcggtgtttatcaagagcttttatgttttgttttgttttttttcctttttttttgagacggagtctcactctgtcgcccaggctggagtgcaatggcaccatcttggctaactgcaacctgcacctcctgggttcaagggattctcctgcctcagcctcccgagttgctgaaattacaggcacgtgccaccacacccgtctaattttttacatttttggtagagatggggtttcaccatgttggctaggctggtctcgaagtcctgacctcgagtaatccacccacctcagcctcccaaactgctgggattacaggagtgagccaccacgcccggcctaagagcttttatcttaatcctgtagtcttcttacagtttctgagtccttttttttttttttttttttttttttgagacggagtctggagtctcgctctgtgacccaggctggagtgtagtggtgcgatctcggcccactgcaagctccgcctcccgggttcacgccattctcctgcctcagcctcccgagtagctgggactacagcgcccgccaccatgcccggctaatttctttttgtatttttagtagagacagggtttcaccctgttagccaggatggtctcgatctcctgacctcatgatccgcccgcctcgacctcccaaagtgctgggattacaggcgtgagccatcttagttctcttttatacatgggtttgaaatgcttcttcttttagaacacattttcctttaataggatatttattgggtttgggttctttgttttttaaaaaatgtacaatgaagtgcaaagaaaaagaaaaaaaaaagtgccataatgtccttgcccagataaccctactaatatttttcttttgaaaaactaaaagtaatatacatgtaagatcgaagcattctaacactgcaaagtatagatcttatttttaagtacattggaataatatgcatgttaggacactattattcaagatgtctttaattttctttctcaaattattcttacagtcttctgtgatcatccatccagaatttaatcatttaaaaaccttcatcttagacgtatacactgctgatgggaatgtaaattacttcagcactgtggaaagcagtttggtgatttcttaaagaatttaaaacagaactgtcatttgaccagcaatcccattactggatatatacccaaaagaatataaatcattctacagtaaaggcaaatgcatatgtatgttcatcgcagcactattcacaatagcaaacacactgaatcaacctaaatagcaattttttaagcaacagtagactggataaagaaaatgtagtacatatacactatggaatgctacacagccacagaaaagaacaagatcatgtcctgtgcaacagcatggatggagctggaggccattatcctaaacaaactaacacaggaacagaaaaccaaacaccacgttctcacttataagtgagagctaaacattgaatacacatggacccaaagaaagtaatagacagcagggcctacttgagggtagagggtgggaggagggtgaggattcgaaaagtacctattaggtactaagcttattacctgggtgatgaaataataacccctgtgacacacaatttatctatagaaccaacctgtacatgtttccttgaaactaaaagtttttttaaaaacacctggccaggcatggtggctcacgcctgtaatcccagcactttgggaggctgaggcaggcagatcacaaggtcaggagttctagaccaacctggccaatatggtgaaacccggtctccactaaaaatacaaaaattagccaggcatggtggcgggcgcctgtagtcccagctactctggaggctgaggcaggagaatcgcttaaacccaggaggcagagcttgcagtgagccgagattgcgccactgcactctggcctgggcaacagagtgagattccgtctcaaaaaaaaacaaaaaacaaaaaaacctgcatcgctcttgagttactttcaattttggaacctttgggaatagaatcatacttttcctctaaactttacaaaacctattttaccatagacagagtctaaaatacatgtttcgcatgtttggttattacaatatgcacacttcagtatcatgccccggttttttccagttggttagaattaggtcttttttttttttttctttttgagacggagtctcactctgtcacccaggctggagtgcagtagggccatctcggctcactgcaacctccacctcccaggcttaagcgattctcctgcctcagtctcctgggtagctgagatcacaggcgtgtgccaccactcccagctaatttttgtgtttttagtaaagacggggttttgccatgttggccaggctggtctcgaacttctggcctctagtgatcttccccctcagcctcccaaagtgctgggattacaggcatcagccaccacgcccaaccagaattaggtctttattaataattattaacccttcaaaaaagcccagttgttcaatagtgttatgtgtttgtccatatcgattttagcaaatattaatattttaattaactgtacatattatattttaaagtaaaacatacatacttcaggaaaggggactcagaaaaaggaaaaaaatacatacatacatgcatacattaaatatattatcaaaagtaagtctttctccaaccctagacccctattttcccagtctccctgtccaaaccaacaagttagcagtttttagaggttttctgtttgtttgtttgtttgtttgtttgtttgttttcacagagtctcactctgttgcccaggctggagggcaatggtgcagtgcaatctcaatctcagctcagggcaacctccacctcccggctcaagcgattctcctgcctcagcctcccaagtagctgggattacaggcgcatgtcaccacacccagctaatttttgtatttttagtagacacggagtttcaccatgttggccaggctggtcttgaactcctgacctcaggtaatccacccgcctcagcctcccaaagtgctgggattacagggatgagccaccatgcctggcccaatttgatttttttttttttttttttgagatggagtcttgctctgtcagccaggatggagtgcagtggcgtgatctcggctcactgcaacctctgcttcccgggttcaaacgattcacctacctcagcctcccgagtagctgggattacaggcgcccaccaccatggctggctaattttttgtgtttttagtagagacagggtttcaccgtgttagccaggatggtctcgatatcctgacctcatgatccgcctgcctcggcctcccaaagtgctgggattgcaggcatgagccaccacgcccggccccaatttgattttttaaagtccacttagcaagattcaatttgattttctgatgagacagagcgtattttcctatgtttataagccagacatatatctttatctgggaacttttttgagtccacagcccaatttttttaatccattggtaagctagtttatatatttataaatacttatgtataaggaaatacacctttgaccttcatataagatgcaaagttattttctttgccaccatcccagattcttacttattttttaaatttgtttatggtatatttttccatgcaaaaagtttttttttttattttttatttttggctagtcaagtgaagcagtgggagtagagaaggaacaaataaatctgtaactggttgtaattaattagttgtaaactccactgcactcggaccagcaccatgcaaaaagttttaaagtctatgtttttttttttttttttttttgagacggagtttcactcttgttgcccaggctgaagtgcagtggcgtgatcttgcctcacctcaacctctgtctcccgggttcaagcgattctcctgcctcagcctcccgagtagctgggattacaggcatgcgccaccacccccagctaattttgtatttttagtagagacggggtttctccatgttggtcaggctggtcttgaacccccgccggccaaaagtctatgtattaaaattaacttgccgggcgcagtggctcacacctgtaatcccagcactttgggaggccgagacgcgtgggtcacctgaggtcaggagttcgagaccagcctgaccaacatggagaaaccacgtgtctacgaaaaacacaaaattagcccggcgtggtggtgcatgcctgtaatcccagctactcaggaggctgagacaggagaatcgtttgaacccagaaggcggaggttgcagtgagccgagattgcgccactgcactccagcctgggcagcaagagcgaaaccgcatctcaaaaaaaaaaaaattaaaattaacttgtcacactactgcctttaggttttgcatcaaaattctttaaaggttacatgtgttttcctgtgatatttttaaagtttttatttaaatcttgaaaccacctaaaatttatgtcactgttccccaggaatgaaggagaaattcagcttttttctcctcccaaatggctacttatcccaacacaagccgtattaattgcatgtaaggctgctgtattctagcacaatgcatgctttcagaaatatttgctaaatgaataaatgaaaagcagtatttgtttattctttctacaaatatgcattgagtaccgactgcatataaccactatgctaggctttgcatataacagtaatgactcagaagccaggaagcatatcatctagcggtgaagataagcattaaataagtaatcatatagcaatattttgaacaggataaaattcctcttacagtcaacactcaaatgtgctggataaaatatgacattttggccgggcgcagtggctcatgcctgtaatcccagcactttgggaggccaaggtaggtggatcacctgaggttaggagtttgagaccagcctgactaacatggtgaaactccgtctctactaaaaatgcaaaagttagccaggtgtagtgtaatcccagctactcaggaggctgaggcaggagaatcgcttgaacctgggaagtggaagttgcaatgagccgagatcacggcatcgcactccagcctgggcgacagagcaagactctgtctcaaaaaaaaaaaaaaaaaaaaaaaagacgttttaaaatgcagaggtgagctcacaagaaaggggtatactgggggcaggtgctaagtgggaactgaaaaaacagaggctgtcctggggcaatctgtcctcctaagtaatattacatataacctggattccaaagggagcaagagacaagcctttgggcaactgtatttaatcaaattgtgatgagtagaaatccatgaaggtttttgggagaaattaaatacagcatctgagacttgagttctagtcctgtttattctaacattgactgtctatgaggtcatggtcaaatagtgtacctttactcttaattccattccctcttgtgcaaaatgagagaattgaagtaaaatatctataaacgacatttcagctctaaaacttgaggattctatgataggatatccctctacaaatttaatttggctgcagtatggatggtggattattacaacagcctcctaactgatacatctgctttcaggcttgctctacctccaaaatcatctttttactgaccacaagaacctttcctgatcaggtctgaattttatcccgcttaaaatttttcaaaggttttccagaactttcagagcctgggggacagggtgtgaacctgtctcaaaaaaaaaaaaaatcctaagtgttaaatgaatgaataaatgtattgaagcaagaagacctggtaaatttagcaacagctctgtagcaaagtaatgcagctctgaattgatacggcagtgctaagaatggaaaggtggtcattcattagtaaataatcgtggaaacacaatctataagacattcaaagagtgtcactgatgaaaggcagaacttagaggagtgaaagttgcagataccaggatgaaatcacttttgtcagaaccaaacaaaatagggccagaaaggcaaaaaggaaagggctcatgtttgtatgtctgagataagaactgtttctgaagactttctaaaaactccacaagagatcacttcacatccttcacacatgtcctcctttgcacagttggcaggtttcagacatatacatatatttctatgacaaggcttatcattctttaggactgcagtaatttagataagaagctctcaaaagaacacttgcccagtaacagcatctccaccaatgaactgatgacagctctggctctgagcctctggaaccaatgaactctgttcattgaacagaactcttgcttctgtttccaagcagctttatttgaacttgccctttttgccaataaaagcttccttttacccttccctcactggacgtacttttggcttgccatttcgtgcatcctgaattctaatattctcttcccatttctgaataaattcaacatatttggagatatttttctccagtcttttttttcccttttttgagactgaaagaggtctgatgcttaggtgcatattttttaactatttagctgtattgagtcttagatgatgtttaccactaaaaaaaaaactaactccaacatctggatcaaatgtgctagggcaagcaagtctacactgtccatagattgtgacctcgttctttaagaatctgatgtgggtggggcatggtggctcatacctgtaatccgagcactttgggaggctgaggcaggaggacagcttgaggtcaggagttcaagaccagactaggcaacatagcaagaacccccatctctagcaaaaagtaaaaaaacaaacaaaaaaaccacacacatctggccatggtggcacgcacctgtagtctcagctactcgggaggctggggtaggaggatggcttgagcccaggagtttgaggttgcagtgagctatgaatgttgccactgcactccagcctgggtgaaaccctgtctccaaaactaagctaaactaaaaaaaaaaaaattctgatgtgactgtgattttactagctcaaacataatgagctacaaagtgagcaaagttcactatgactacaaatgaaaagttgaaagaaatccattgtcggcagggcgcggtggctcacgcctgtaatcccagcactttgggaggctggggcgggtggatcaccagaggtcaggagttcgagaccagcctgaccaatatggtgaaaccccgtctgtactaaaaatacaaaaattacctgggcatggtgacaggcacctgtaatcccagctactcaggaggctgaggcaggagaatcgcttgagccggggaggctgaggttgcagtgaggcgagatcgtgccactgcactccagcctgggcaacagagcggaaggaaggaagaaatgaaggagggagggagggagggagggagggaaccattatctgttgaatgcctagtatgtgccagaaagccagggaaataaatgaaaagtgcaattggagcttgcaaaaaggaaggtcaagatgctaaagaaggtttagcggaaactgaactgggatttgatcatattaattaatatgaacaaaaagagagaaaagaatgtagtttgtaggccaggtagaagaagaactaattttatagaagaaaaacaaatctaaatgttatatcttttatttctcttgtcaaaaacttgtagtccaattatttttaactccttatgtatttccattttcagtctgtctacaaggaaaactgactcgaggtaattaaggatagagatcaaggggcagattttatttgtatggtggtaaagttcaaagggctctttgctccacaatcgatccactaagaccaaaaactttaggtgtactctagagtgtcacatccaagtatttaaaccattaaatttgggtggacaaaggggaggatgtggaaagggaaatgagataccaaaaactgatagatgtttaaaaaagaggtagtttggaaactaccttttttaagactgacaattaggaagatacatttttggcagtatataccactgtttcactctaaaatcaccgttgaactgttatgtaactttcatctatttatttaattactgaatccatgcttcaacaacccaagcctaagtaacaccaaataaaggactgagattgaggctttttttttttttttttgagacaaggtctcgctctgtcgcccaggctggagggcagttcttcgctcactacctctgcctcctaggctcaagctgggactacagacgcgcgccgccccgcccaactaattttttcttcttcttttttttttttttttttgagagtctcgctctgtcgcccaggctggagtgcagtggcactatctcggctcactgcaacctccgtctcccaggttcaagcgattctcctgcctcagcttcccgggtagctgggactacaggtgcgcgccaacactctgtctaatttttgtatttttagtagagacggggtttcaccatgctggccaggctggtctccaactcccgacctcaggcgattctcccgcctcggcctcccaaagtgctgggattacagtcgtgagccaccgcgcccggcctagttaattttttaatttaatttttttgtggagacgaggtctcactattgcccaggctaatctcgaacttctgggctaagcgatcctcctgcctcgagcctcccaaattgttgggcttacaggcgtgagccaccgcacctggcggaacatttctactctaatgaaaattataacagtttttacagttttcctagctcccaactgtacctataagttcagagtccatttaatgtacttgtaataattcaggctggtgttttattatcttttctacattattaccctctcaaagaacaaaagcaggtaactcctataaagtctaatagtctgaaaattgtagtgtccgttttctctttagccataaagttttgcttaaatgctgagtcgcgttttaatgctctactagatgtacatataagaatactaaatggagggaaagtaatttcttttttttttttgagacggggtcactcaggctggagtgccagtgcgcgattacagctcaccggagcctctcgacctccccaggctcaagtgatcctgccacctcagcctccccaatagctgggactacagacacgcgccaccacgcccggctaatttttttgtatttttcttagagactgggttttgccatgttgcccaaactggtcttgaactcctgggctcaagcgacctgcctgcctcagccccgcaaagtgctgggattacaggcgtgagccaccgcgcccggccataattttatcttaatcacttttttccaggaactattacgtttcaaagaatgaggaattaactgacaacacttcaggttcaacatgtaaatagggacgctcatttgagaataaatcacattttgtcacagttgtattgaaaaatcggtatttctgaacgtcattttaccattacggaagtataactcagacacaataaaatgtttaatatacggctaggattaggttaaggtgagagaggcgcctagagcacaaattttaaggaggcaagaccacgaccccgaccgtgagcgcctgcttaacatttgctccccaggcgcttcccactccccagccgggccccgtcctagttcaatcaatcaagaaaatcaagagtacgaaggccttgtgcggtctggacggtctttccacgtctcaggtgcagttaagccagtcacactccatccacctacccgccatgaaaaattatttgttcagcaggaattgccaagtcaaatacagccatataaagccccccttcgacgtgccaggtgtgcctccggagagcagccaccaaaaccaccagcagcccgtccgcgccgggaaatgcgccctcgctggcttacggggccactcagacagttctaagtgcctttccctgtaacgcagaggcacacctgccttccctcgggaaacagcggcccgacgtgcgcccgcaacgcgtaaagcgtcaacagctgaaaccctcggaatcctccccgaacagaaacaaccaccgctgccagctgcgcgctcgggggaaaagacgttgcgcccccgccgactgccggtttcccgggcgcgagcccggatccaggtggtcagtcccggtacgcaaccacggcgagaacccggccctgctaagggagaagggaagccgtttcccgcgggcttcatacagacacggtgctaagacagcccgagtcgcggaccagtccaacaggcaggcagatcagtgggcagcccgcgtgc

SEQ ID NO: 22 - RNA, 111 nt, synthetic construct
  source 1..111: mol_type = other RNA
gggcgtgtctcggcacccctgtttcagagctatgctggaaacagcatagcaagttgaaataaggctagtccgttatcaacttgaaaaagtggcaccgagtcggtgcttttt

SEQ ID NO: 23 - RNA, 111 nt, synthetic construct
  source 1..111: mol_type = other RNA
gagctcccgggttccggagcgtttcagagctatgctggaaacagcatagcaagttgaaataaggctagtccgttatcaacttgaaaaagtggcaccgagtcggtgcttttt

SEQ ID NO: 24 - RNA, 111 nt, synthetic construct
  source 1..111: mol_type = other RNA
ggggcggctactcgagttcagtttcagagctatgctggaaacagcatagcaagttgaaataaggctagtccgttatcaacttgaaaaagtggcaccgagtcggtgcttttt

SEQ ID NO: 25 - RNA, 111 nt, synthetic construct
  source 1..111: mol_type = other RNA
gaattccgggtcaatgggcggtttcagagctatgctggaaacagcatagcaagttgaaataaggctagtccgttatcaacttgaaaaagtggcaccgagtcggtgcttttt

SEQ ID NO: 26 - DNA, 3411 bp, synthetic construct
  source 1..3411: mol_type = other DNA
gtggtggcgggcgcctgtagtcccagctactcgggactacaggagaatcgcttgaacccaagaggcggagtgttgcagtgagccaagatcgctccactgcactccagtctggcgacagagcgagaccccgtcttaaaaaaaacaaaacaaaacaaaaaaaaaacctttgctttcccgccttccaagaggaaggggcgcatctctgaggaaacaagcccggggaacccgcagcccggacggcatcctctcatctacacaacccgaggacatctccctcagctcccagaggacggcggaccatggtcaaggtcctctttccggcctttcacaactccaagccccgccccacaaaaccccctgcagtcctaccaagtcactcccatggcgtcgggccaggaaggtggcagaatcacacagtagaaacgcagggttccctgtccgcccacagaaactccttcagttccccaacacaaccttctgtctcagaagggactccggaaatacgtaaaaacagagcgccgggtccacgccttccccaaggaatggggcgcgtctcggcacctaggctagcgatcctctagattcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgactcgagagcgctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccggagcgactaacttctccctgctgaagcaggccggcgatgtagaagagaacccgggccctaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgaggcacccctcgatgacgcctccactgacatcatctgcggattcccgccccgaggtttctaaatccagacattcccgtttggctgagcactctaggcctacatccatgaagctaggagagcgacactcaaagactgcactattgagagaagctaacgttaaaggcagtgaatatattcgggagtacagctccggaacgcgtgagctcttttagtgggaggggcggcgctgatggcgcttctggcctccgaatgctagggggcgctgtgatcgccgggcggcctcttgggcgctgggagtccaccgcgcaagcgcatcctggcctttcttcagtccccacgtgcgatccttcccggcaactttttcgagaaaaatgcccaaattcaaggcggcccgtggggtggggggtcaggaaaaacatgcgcccctggccgatcagatcctggctgggaatgcggtgcgggcgggggtccgggagaagcggcggggtcgcgggacaggagaagcggaggaagagtatgtggggccccggctgagccgacggattttgcagcaagcacggcagcaacaggaggaactcgaggccgagcatgggactggggacaagcccgcggcgccgcgggaacgcaccacgcggctgggtgagtgtctgggatgaggtccgaggaagacagtggccagtagtggggcggggtgggcagctaaaaagtgtctggcaggggaattgcttcgagtcaacaaa

SEQ ID NO: 27 - DNA, 3411 bp, synthetic construct
  source 1..3411: mol_type = other DNA
gtggtggcgggcgcctgtagtcccagctactcgggactacaggagaatcgcttgaacccaagaggcggagtgttgcagtgagccaagatcgctccactgcactccagtctggcgacagagcgagaccccgtcttaaaaaaaacaaaacaaaacaaaaaaaaaacctttgctttcccgccttccaagaggaaggggcgcatctctgaggaaacaagcccggggaacccgcagcccggacggcatcctctcatctacacaacccgaggacatctccctcagctcccagaggacggcggaccatggtcaaggtcctctttccggcctttcacaactccaagccccgccccacaaaaccccctgcagtcctaccaagtcactcccatggcgtcgggccaggaaggtggcagaatcacacagtagaaacgcagggttccctgtccgcccacagaaactccttcagttccccaacacaaccttctgtctcagaagggactccggaaatacgtaaaaacagagcgccgggtccacgccttccccaaggaatggggcgcgtctcggcacccctcgatgacgcctccactgacatcatctgcggattcccgccccgaggtttctaaatccagacattcccgtttggctgagcactctaggcctacatccatgaagctaggagagcgacactcaaagactgcactattgagagaagctaacgttaaaggcagtgaatatattcgggagtacagctccggaacgcgctaggctagcgatcctctagattcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgactcgagagcgctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccggagcgactaacttctccctgctgaagcaggccggcgatgtagaagagaacccgggccctaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgagcgcgtgagctcttttagtgggaggggcggcgctgatggcgcttctggcctccgaatgctagggggcgctgtgatcgccgggcggcctcttgggcgctgggagtccaccgcgcaagcgcatcctggcctttcttcagtccccacgtgcgatccttcccggcaactttttcgagaaaaatgcccaaattcaaggcggcccgtggggtggggggtcaggaaaaacatgcgcccctggccgatcagatcctggctgggaatgcggtgcgggcgggggtccgggagaagcggcggggtcgcgggacaggagaagcggaggaagagtatgtggggccccggctgagccgacggattttgcagcaagcacggcagcaacaggaggaactcgaggccgagcatgggactggggacaagcccgcggcgccgcgggaacgcaccacgcggctgggtgagtgtctgggatgaggtccgaggaagacagtggccagtagtggggcggggtgggcagctaaaaagtgtctggcaggggaattgcttcgagtcaacaaa

SEQ ID NO: 28 - DNA, 3393 bp, synthetic construct
  source 1..3393: mol_type = other DNA
tccggcctcgcaaccgaccaccccttctgcaggagaggtacataatggtcgcaaagagcaggcgaaccgggcagagatcgctcgctcgctcgcagactgagcgaatccggactcacccgtctccttcgccaagtgataaaacttgtctcgtcggctcttgccaactttgcccttcttgcccatggtggaaagggggagtatccctcaatctggggagaaagtagaagttgggaacgtctctttccggagcaccagattccacttccagtttcccacttccagtttcccacttcccgcagcaattccaccccgcgcccctctccgcacactacctagacccagagccgctttctccacacttggaaccgcacaagtatgcagctaactacttccgcttccgcttgcgtcccctgcgtccctggctcgaggttttccgccattttgagtgtggccctagattgttctcaatactctgtccttgggttttgtaagttgaaagttgagactaggaaggcatatcacaagaactataaacagagtttcaatgaggcaacactgaggaggagttctactccttcctcatccccttccaaagcacctgaactctagctaggctagcgatcctctagattcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgactcgagagcgctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccggagcgactaacttctccctgctgaagcaggccggcgatgtagaagagaacccgggccctaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgagctagaagccgcccctcccatcatggttcccttagtgtggtctcgccgcacaacccgcccatgtacacggaattcttctctgcctggtcttcaggtcccaatcctccgcttccgcgcttgcgcgccaagacggctcggatgccggcggtctctgctgaagagagaagatggcgcttgacggaccagagcaggtatggcgggtgcagtggcggcccggcaggttacggggctgggtgcggagcgagcgtgatctgagtggagagcgggccggggcaggagcgtcgggtgggtcggaggtgcctggaccagtccatgctggacgctgcctgcgaccggatctgctgtgtcagcgccgccctcggtgagtcagggcaaggctgggagaggaagacgcggtagaagcggagtgagtgagggaagcgatgggcgcgggaatggccggcccacgggtcgcaggagacgggacgccagtcctttggctccgttccgctggctccttcgtcagtactgacacctcgggcttgtagagcacttcacgcagtgcaaagcgccccccgtctatatcatatcgcctctcggtcctcctaaaagtcgtatgaggtaggcggcgttcccattcttttattttagtcctgggaaaatggagggttccaagggagggagggattagaacc

SEQ ID NO: 29 - DNA, 3393 bp, synthetic construct
  source 1..3393: mol_type = other DNA
tccggcctcgcaaccgaccaccccttctgcaggagaggtacataatggtcgcaaagagcaggcgaaccgggcagagatcgctcgctcgctcgcagactgagcgaatccggactcacccgtctccttcgccaagtgataaaacttgtctcgtcggctcttgccaactttgcccttcttgcccatggtggaaagggggagtatccctcaatctggggagaaagtagaagttgggaacgtctctttccggagcaccagattccacttccagtttcccacttccagtttcccacttcccgcagcaattccaccccgcgcccctctccgcacactacctagacccagagccgctttctccacacttggaaccgcacaagtatgcagctaactacttccgcttccgcttgcgtcccctgcgtccctggctcgaggttttccgccattttgagtgtggccctagattgttctcaatactctgtccttgggttttgtaagttgaaagttgagactaggaaggcatatcacaagaactataaacagagtttcaatgaggcaacactgaggaggagttctactccttcctcatccccttccaaagcacctgaactctagaagccgcccctcccatcatggttcccttagtgtggtctcgccgcacaacccgcccatgtacctaggctagcgatcctctagattcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgactcgagagcgctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccggagcgactaacttctccctgctgaagcaggccggcgatgtagaagagaacccgggccctaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgaggtacacggaattcttctctgcctggtcttcaggtcccaatcctccgcttccgcgcttgcgcgccaagacggctcggatgccggcggtctctgctgaagagagaagatggcgcttgacggaccagagcaggtatggcgggtgcagtggcggcccggcaggttacggggctgggtgcggagcgagcgtgatctgagtggagagcgggccggggcaggagcgtcgggtgggtcggaggtgcctggaccagtccatgctggacgctgcctgcgaccggatctgctgtgtcagcgccgccctcggtgagtcagggcaaggctgggagaggaagacgcggtagaagcggagtgagtgagggaagcgatgggcgcgggaatggccggcccacgggtcgcaggagacgggacgccagtcctttggctccgttccgctggctccttcgtcagtactgacacctcgggcttgtagagcacttcacgcagtgcaaagcgccccccgtctatatcatatcgcctctcggtcctcctaaaagtcgtatgaggtaggcggcgttcccattcttttattttagtcctgggaaaatggagggttccaagggagggagggattagaacc

SEQ ID NO: 30 - protein, 335 aa, synthetic construct
MARLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSPKKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 31 - DNA, 50 bp, synthetic construct
  source 1..50: mol_type = other DNA
acacgacgctcttccgatctacataacggtgtggaaaggacgaaacaccg

SEQ ID NO: 32 - DNA, 50 bp, synthetic construct
  source 1..50: mol_type = other DNA
acacgacgctcttccgatcttagttacggtgtggaaaggacgaaacaccg

SEQ ID NO: 33 - DNA, 58 bp, synthetic construct
  source 1..58: mol_type = other DNA
aatgatacggcgaccaccgagatctacactctttccctacacgacgctcttccgatct

SEQ ID NO: 34 - DNA, 45 bp, synthetic construct
  source 1..45: mol_type = other DNA
caagcagaagacggcatacgagatggtctaaccagagagagccag

SEQ ID NO: 35 - DNA, 2579 bp, synthetic construct
  source 1..2579: mol_type = other DNA
gtggtggcgggcgcctgtagtcccagctactcgggactacaggagaatcgcttgaacccaagaggcggagtgttgcagtgagccaagatcgctccactgcactccagtctggcgacagagcgagaccccgtcttaaaaaaaacaaaacaaaacaaaaaaaaaacctttgctttcccgccttccaagaggaaggggcgcatctctgaggaaacaagcccggggaacccgcagcccggacggcatcctctcatctacacaacccgaggacatctccctcagctcccagaggacggcggaccatggtcaaggtcctctttccggcctttcacaactccaagccccgccccacaaaaccccctgcagtcctaccaagtcactcccatggcgtcgggccaggaaggtggcagaatcacacagtagaaacgcagggttccctgtccgcccacagaaactccttcagttccccaacacaaccttctgtctcagaagggactccggaaatacgtaaaaacagagcgccgggtccacgccttccccaaggaatggggcgcgtctcgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgcgatcgcgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagatgaaccggtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcccctcgatgacgcctccactgacatcatctgcggattcccgccccgaggtttctaaatccagacattcccgtttggctgagcactctaggcctacatccatgaagctaggagagcgacactcaaagactgcactattgagagaagctaacgttaaaggcagtgaatatattcgggagtacagctccggaacgcgtgagctcttttagtgggaggggcggcgctgatggcgcttctggcctccgaatgctagggggcgctgtgatcgccgggcggcctcttgggcgctgggagtccaccgcgcaagcgcatcctggcctttcttcagtccccacgtgcgatccttcccggcaactttttcgagaaaaatgcccaaattcaaggcggcccgtggggtggggggtcaggaaaaacatgcgcccctggccgatcagatcctggctgggaatgcggtgcgggcgggggtccgggagaagcggcggggtcgcgggacaggagaagcggaggaagagtatgtggggccccggctgagccgacggattttgcagcaagcacggcagcaacaggaggaactcgaggccgagcatgggactggggacaagcccgcggcgccgcgggaacgcaccacgcggctgggtgagtgtctgggatgaggtccgaggaagacagtggccagtagtggggcggggtgggcagctaaaaagtgtctggcaggggaattgcttcgagtcaacaa

SEQ ID NO: 36 - DNA, 2566 bp, synthetic construct
  source 1..2566: mol_type = other DNA
tccggcctcgcaaccgaccaccccttctgcaggagaggtacataatggtcgcaaagagcaggcgaaccgggcagagatcgctcgctcgctcgcagactgagcgaatccggactcacccgtctccttcgccaagtgataaaacttgtctcgtcggctcttgccaactttgcccttcttgcccatggtggaaagggggagtatccctcaatctggggagaaagtagaagttgggaacgtctctttccggagcaccagattccacttccagtttcccacttccagtttcccacttcccgcagcaattccaccccgcgcccctctccgcacactacctagacccagagccgctttctccacacttggaaccgcacaagtatgcagctaactacttccgcttccgcttgcgtcccctgcgtccctggctcgaggttttccgccattttgagtgtggccctagattgttctcaatactctgtccttgggttttgtaagttgaaagttgagactaggaaggcatatcacaagaactataaacagagtttcaatgaggcaacactgaggaggagttctactccttcctcatccccttccaaagcacctgaactctctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgcgatcgcgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagatgaaccggtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagaagccgcccctcccatcatggttcccttagtgtggtctcgccgcacaacccgcccatgtacacggaattcttctctgcctggtcttcaggtcccaatcctccgcttccgcgcttgcgcgccaagacggctcggatgccggcggtctctgctgaagagagaagatggcgcttgacggaccagagcaggtatggcgggtgcagtggcggcccggcaggttacggggctgggtgcggagcgagcgtgatctgagtggagagcgggccggggcaggagcgtcgggtgggtcggaggtgcctggaccagtccatgctggacgctgcctgcgaccggatctgctgtgtcagcgccgccctcggtgagtcagggcaaggctgggagaggaagacgcggtagaagcggagtgagtgagggaagcgatgggcgcgggaatggccggcccacgggtcgcaggagacgggacgccagtcctttggctccgttccgctggctccttcgtcagtactgacacctcgggcttgtagagcacttcacgcagtgcaaagcgccccccgtctatatcatatcgcctctcggtcctcctaaaagtcgtatgaggtaggcggcgttcccattcttttattttagtcctgggaaaatggagggttccaagggagggagggattagaacc

SEQ ID NO: 37 - DNA, 11363 bp, synthetic construct
  source 1..11363: mol_type = other DNA
ttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagggatccggagcgactaatttttccctgctgaagcaggccggcgatgtagaagaaaacccgggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaattcacgcgttaagtcgacgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgattccggggcggggcgaatcttttaaaagaaaaggggggattggggggtacagtgcaggggaaagaatagtagacataatagcaacagacatacaaactaaagaattacaaaaacaaattacaaaaattcaaaattttgattccggggcggggcgaatcgattccggggcggggcgaatcctcgagtttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtccctagtggccccactgtggtttcagagctatgctggaaacagcatagcaagttgaaataaggctagtccgttatcaacttgaaaaagtggcaccgagtcggtgctttttttaattaagggggcccgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacctctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggggagcgcttacgtagaaggagaggccaccatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaac

SEQ ID NO: 38 - DNA, 2937 bp, synthetic construct
  source 1..2937: mol_type = other DNA
tgctttctctgaccagcattctctcccctgggcctgtgccgctttctgtctgtagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgcgatcgcgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagatgaaccggtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggacttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 39 - RNA, 20 nt, synthetic construct
  source 1..20: mol_type = other RNA
gggcgtgtctcggcacccct

SEQ ID NO: 40 - RNA, 20 nt, synthetic construct
  source 1..20: mol_type = other RNA
gaattccgggtcaatgggcg

SEQ ID NO: 41 - RNA, 21 nt, synthetic construct
  source 1..21: mol_type = other RNA
gtccctagtggccccactgtg

SEQ ID NO: 42 - DNA, 20 bp, synthetic construct
  source 1..20: mol_type = other DNA
gtctggtctatctgcctggc

SEQ ID NO: 43 - DNA, 20 bp, synthetic construct
  source 1..20: mol_type = other DNA
aaacagacacaagcgggtct

SEQ ID NO: 44 - DNA, 20 bp, synthetic construct
  source 1..20: mol_type = other DNA
tcctgctgcaatcggagaag

SEQ ID NO: 45 - DNA, 20 bp, synthetic construct
  source 1..20: mol_type = other DNA
gttacgaaccatccccctgg

SEQ ID NO: 46 - DNA, 20 bp, synthetic construct
  source 1..20: mol_type = other DNA
acccttcctagctccctctg

SEQ ID NO: 47 - DNA, 20 bp, synthetic construct
  source 1..20: mol_type = other DNA
ggtagccaggatgtcgaagg

SEQ ID NO: 48 - DNA, 20 bp, synthetic construct
  source 1..20: mol_type = other DNA
ccggcgtctactatgtggac

SEQ ID NO: 49 - DNA, 21 bp, synthetic construct
  source 1..21: mol_type = other DNA
ggtctaaccagagagagccag

(19) United States
(12) Patent Application Publication
(10) Pub. No.: US 2023/0151392 A1
(43) Pub. Date: May 18, 2023

(54) SAFE HARBOR LOCI

(71) Applicant: Bit Bio Limited, Cambridge (GB)

(72) Inventors: Julian JUDE, Vienna (AT); Anatoly VASILYEV, Vienna (AT); Adam KREJCI, Vienna (AT); Tilmann BUERCKSTUEMMER, Vienna (AT); Anke LOREGGER, Vienna (AT); Lukas BADERTSCHER, Vienna (AT)

(21) Appl. No.: 17/815,928

(22) Filed: Jul. 28, 2022

Related U.S. Application Data
(63) Continuation of application No. PCT/EP2021/052101, filed on Jan. 29, 2021.

(30) Foreign Application Priority Data
Jan. 30, 2020 (EP) 20154496.2

(51) Int. Cl. C12N 15/90; C12N 15/11; C12N 9/22; C12N 5/0793; C12N 5/077

(52) U.S. Cl. CPC C12N 15/907; C12N 5/0619; C12N 5/0657; C12N 9/22; C12N 15/11; C12N 2310/20; C12N 2506/45; C12N 2800/80

(57) ABSTRACT

An isolated mammalian host cell comprising a heterologous gene of interest (GOI) chromosomally integrated at a target site within an intergenic region between a pair of adjacent essential genes.

### CROSS-REFERENCE TO RELATED APPLICATIONS

This application is a continuation of PCT/EP2021/052101, filed on Jan. 29, 2021, which claims the benefit of and priority to European Patent Application No. 20154496.2, filed Jan. 30, 2020, the disclosures of each of which are hereby incorporated by reference in their entireties for all purposes.

### REFERENCE TO A SEQUENCE LISTING XML

This application contains a Sequence Listing in XML format. The Sequence Listing XML is incorporated herein by reference. Said XML file, created on Jul. 25, 2022, is named BBIO-007WOC1_SL.xml and is 113,663 in size.

### FIELD OF THE INVENTION

The invention relates to a recombinant host cell and methods of expressing a gene of interest (GOI) from a host cell. The invention relates particularly to methods of improving a host cell’s capacity to stably express a GOI over a long period of time, and gene therapy employing the recombinant host cell.

### BACKGROUND OF THE INVENTION

Cell and gene therapy relies on the insertion of transgenes in target cells. Insertion can happen in many different ways, using for instance transposons or lentiviral vectors. Transgene insertion may also happen via homology-directed repair, facilitated by programmable nucleases such as Zinc finger nucleases (ZFNs), transcriptional activator-like effector nucleases (TALENs), Clustered Regularly Interspaced Short Palindromic Repeats (CRIPSRs), homing endonucleases and meganucleases. Popular transgenes include genes whose function is defective, but also chimeric antigen receptors (for CAR-T therapy), T cell receptors (for TCR therapy) or cDNAs whose function is defective in patients.

Stable expression in eukaryotic cell lines is useful for numerous projects. For example, it supports recombinant protein production over a prolonged period in a cell culture, or it helps in transgenic eukaryotes for optimization of protein productions at high yields. Maintaining stable expression of transgenes is important to ensure functionality. Stable expression implies insertion into the genome.

Randomly inserted genes are subject to position effects and silencing, making their expression unreliable and unpredictable. Targeted insertion in safe-harbor sites has made some progress. To this end, so called safe harbour loci can be targeted e.g., AAVS1 in human (also known as PPP1R12C) locus, a well-validated “safe harbor” in the human genome, or ROSA26 in mouse and human, that are actively transcribed.

Safe harbor sites are defined based on their position relative to contiguous coding genes, microRNAs and ultra-conserved regions and, for example understood to meet the following criteria: (i) distance of at least 50 kb from the 5’ end of any gene, (ii) distance of at least 300 kb from any cancer-related gene, (iii) distance of at least 300 kb from any microRNA (miRNA), (iv) location outside a transcription unit and (v) location outside ultra-conserved regions (UCRs) of the human genome (Bejerano G, et al.. Science. 2004;304:1321-1325; Pellenz S, et al., Hum Gene Ther. 2019;30(7):814-828).

Browning Jill et al. (Molecular Biology Reports 2019, 47(2):1491-1498) describe CRISPR targeting of the murine Hipp11 intergenic region to support inducible human transgene expression.

Keiichiro Suzuki et al. (Nature 2016, 540(7631):144-149) describe genome editing via CRISPR/Cas9 mediated homology-independent targeted integration.

Dahodwala Hussain et al. (Current Opinion in Biotechnology 2019, 60: 128-137) describe strategies to predict CHO cell line instability.

Nevertheless, transgene silencing remains an important problem that is not easily addressed and that hampers the feasibility of the above-mentioned approaches. Silencing is a phenomenon that is implemented by establishing repressive marks on DNA or histones e.g., trimethylation of histone H3 on lysine 9 or 27. When silencing occurs, these repressive chromatin marks are spread over a certain region, typically at least several kilobases of DNA sequence (Mlambo et al. Nucleic Acids Res. 2018; 46(9):4456-4468) and may not only affect the transgene itself, but also adjacent regions.

### SUMMARY OF THE INVENTION

It is the objective to overcome transgene silencing by selecting safe harbour loci in the genome that cannot be silenced. Another objective of the invention is to provide transgenic host cells with improved gene expression capabilities over a prolonged period of time.

The object is solved by the subject matter as claimed.

Provided herein is an isolated mammalian host cell comprising a heterologous gene of interest (GOI) chromosomally integrated at a target site within an intergenic region between a pair of adjacent essential genes.

The invention provides for an isolated mammalian host cell comprising a heterologous gene of interest (GOI) chromosomally integrated at a target site within an intergenic region between a pair of adjacent essential genes, wherein each of the adjacent essential genes of said pair has a transcription start site (TSS) and the distance between the TSSs is less than any one of 20.000, 15.000, 10.000, 5.000, 2.500, 1.000, 900, 800, 700, 600, 500, or 400 nucleotides (nt).

Specifically, the distance between the respective TSSs is at least any one of 80, 90, 100, 110, 120, 130, 140, 150, 160, 170, 180, 190, 200, 210, 220, 230, 240, 250, 260, 270, 280, 290 300, 310, 320, 330, or 340 nt.

Chromosomal integration is specifically obtained by one or more genetic modifications of the host cell chromosome within the intergenic region and/or at the target site.

Specifically, said one or more genetic modifications comprise an insertion or knock-in of the GOI or an expression construct comprising the GOI.

Specifically, the host cell is a diploid cell (which term is herein understood to encompass a near-diploid cell), such as containing two copies of each chromosome.

Specifically, the isolated host cell is provided in a host cell culture or provided as a pharmaceutical preparation or a donor cell for cell therapy.

Specifically, the adjacent essential genes are contiguous coding genes only separated by an intergenic region, and/or positioned such that the intergenic region between the two adjacent essential genes consists of a contiguous non-coding nucleotide sequence.

Specifically, the intergenic region described herein is understood to comprise or consist of a genomic safe harbor. Specifically, the safe harbor comprises a target site or locus for GOI insertion. The target site can be a randomly chosen site within the intergenic region, or a predetermined site.

Therefore, the intergenic region as described herein is understood to be the polynucleotide sequence between the adjacent essential genes, before a transgene (e.g., the GOI or an expression construct comprising the GOI) is inserted at the target site. In other words, the intergenic region is understood to comprise a certain contiguous sequence which is positioned between the adjacent essential genes that does not comprise a transgene. Where the cell is engineered to comprise a transgene within the intergenic region, the intergenic region is understood to comprise or consist of the polynucleotide sequence which identifies the region between the adjacent essential genes without the transgene, i.e., comprising or composed of two parts of the intergenic region flanking the 5’-end and 3’-end of the transgene, which parts are otherwise fused together, thereby obtaining a contiguous sequence comprising or composed of the nucleotide sequence of the flanking parts.

Specifically, the length of the intergenic region is less than any one of 20.000, 15.000, 10.000, 5.000, 2.500, 1.000, 900, 800, 700, 600, 500, or 400 nt.

Specifically, the intergenic region does not comprise a gene or part of a gene.

Specifically, the intergenic region is non-coding.

Specifically, at least any one of 60%, 70%, 80%, 85%, 90%, 91%, 92%, 93%, 94%, 95%, 96%, 97%, 98%, 99%, or 100% of the intergenic region consists of a polynucleotide of human origin, such as a human polynucleotide. Specifically, the intergenic region comprises a contiguous part of any one of SEQ ID NO:1-21, which is at least any one of 60%, 70%, 80%, 85%, 90%, 91%, 92%, 93%, 94%, 95%, 96%, 97%, 98%, 99%, or 100% of the full-length of the respective sequence of SEQ ID NO:1-21.

Specifically, the intergenic region comprises or consists of the nucleic acid sequence which is at least any one of 90%, 91%, 92%, 93%, 94%, 95%, 96%, 97%, 98%, 99%, or 100% identical to any one of SEQ ID NO:1-21.

According to a specific embodiment, the intergenic region comprises or consists of a polynucleotide which is of human origin, such as a human polynucleotide which is 100% identical to any one of SEQ ID NO:1-21.

Specifically, the intergenic region comprises or consists of a polynucleotide which is a naturally-occurring mammalian or human variant of any one of SEQ ID NO:1-21, such as e.g., a polynucleotide identified by any one of SEQ ID NO:1-21, wherein the polynucleotide sequence is modified by one or more single-nucleotide polymorphisms that naturally-occur within the polynucleotide sequence in the human genome.

Specifically, each of the essential genes is of human origin and/or has at least any one of 85%, 90%, 95%, 96%, 97%, 98%, 99% sequence identity to the respective human gene sequence, or is 100% to the respective human gene sequence.

Specifically, the essential genes are human genes or their natural variants originating from different human beings, or their analogs originating from different mammalian species, such as mouse, hamster, rat, dog or pig. The sequence of an essential gene is largely conserved, with a sequence identity of at least 80% or 85% or 90% of corresponding genes in non-human animals as compared to human beings. For example, the nucleotide sequence of MED20 from human is 93% identical to the corresponding gene in hamster (CHO), 90% identical to the corresponding gene in mouse, 99% identical to the corresponding gene in dog, and 98% identical to the corresponding gene in pig.

Specifically, each of the essential genes of said pair of essential genes is known to express proteins at a high expression level. Essential genes have core functions in the cell and are often highly conserved between species. They are defined as genes whose inactivation or deletion results in the death of the cell.

Specifically, the pair of essential genes is a pair consisting of a first and a second gene, wherein
i) the first gene is NFS1 and the second gene is ROMO1; or
ii) the first gene is MED22 and the second gene is RPLA7; or
iii) the first gene is DDX51 and the second gene is NOC4L; or
iv) the first gene is CENPK and the second gene is PPWD1; or
v) the first gene is ORC1 and the second gene is PRPF38A; or
vi) the first gene is POLR3K and the second gene is SNRNP25; or
vii) the first gene is COPE and the second gene is DDX49; or
viii) the first gene is FTSJ3 and the second gene is PSMC5; or
ix) the first gene is MED20 and the second gene is BYSL; or
x) the first gene is AURKA and the second gene is CSTF1; or
xi) the first gene is NUP88 and the second gene is RPAIN; or
xii) the first gene is ATP6V1D and the second gene is EIF2S1; or
xiii) the first gene is POLR2I and the second gene is TBCB; or
xiv) the first gene is UFD1 and the second gene is CDC45; or
xv) the first gene is CCDC115 and the second gene is IMP4; or
xvi) the first gene is NAA50 and the second gene is ATP6V1A; or
xvii) the first gene is SART3 and the second gene is ISCU; or
xviii) the first gene is C1 orf109 and the second gene is CDCA8; or
xix) the first gene is POLR3A and the second gene is RPS24; or
xx) the first gene is RPS16 and the second gene is SUPT5H; or
xxi) the first gene is RPS29 and the second gene is LRR1.

Each of the essential genes referred to herein is characterized by an Entrez Gene ID number. Entrez Gene is National Center for Biotechnology Information (NCBI)’s database for gene-specific information generating unique integers (GeneID) as stable identifiers for genes (Maglott et al. Nucleic Acids Res. 2011; 39 (Database issue):D52-D57). Each of the genes has a transcription start site (TSS) which denotes the start of transcription initiated by the promoter of the respective gene. The TSS in a nucleotide sequence can be identified using public databases such as RefSeq or FANTOM.

Specifically, the pair of essential genes and the respective intergenic region is characterized as shown in Table 1.

TABLE 1

Exemplary pairs of essential genes and intergenic regions comprising a target site.

| Chr | genel | GenelD gene1 | TSS gene1 | gene2 | GeneID gene2 | TSS gene2 | Intergenic Region, nt* | Intergenic region start | Intergenic region end | SEQ ID NO |
|---|---|---|---|---|---|---|---|---|---|---|
| chr20 | NFS1 | 9054 | 35699348 | ROMO1 | 140823 | 35699401 | 52 | 35699349 | 35699400 | 1 |
| chr9 | MED22 | 6837 | 133348090 | RPL7A | 6130 | 133348210 | 119 | 133348091 | 133348209 | 2 |
| chr12 | DDX51 | 317781 | 132144324 | NOC4L | 79050 | 132144450 | 125 | 132144325 | 132144449 | 3 |
| chr5 | CENPK | 64105 | 65563146 | PPWD1 | 23398 | 65563288 | 141 | 65563147 | 65563287 | 4 |
| chr1 | ORC1 | 4998 | 52404410 | PRPF38A | 84950 | 52404606 | 195 | 52404411 | 52404605 | 5 |
| chr16 | POLR3K | 51728 | 53618 | SNRNP25 | 79622 | 53858 | 239 | 53619 | 53857 | 6 |
| chr19 | COPE | 11316 | 18919374 | DDX49 | 54555 | 18919716 | 341 | 18919375 | 18919715 | 7 |
| chr17 | FTSJ3 | 117246 | 63827092 | PSMC5 | 5705 | 63827436 | 343 | 63827093 | 63827435 | 8 |
| chr6 | MED20 | 9477 | 41921128 | BYSL | 705 | 41921500 | 371 | 41921129 | 41921499 | 9 |
| chr20 | AURKA | 6790 | 56392204 | CSTF1 | 1477 | 56392646 | 441 | 56392205 | 56392645 | 10 |
| chr17 | NUP88 | 4927 | 5419662 | RPAIN | 84268 | 5420190 | 527 | 5419663 | 5420189 | 11 |
| chr14 | ATP6V1D | 51382 | 67359791 | EIF2S1 | 1965 | 67360338 | 546 | 67359792 | 67360337 | 12 |
| chr19 | POLR2I | 5438 | 36114890 | TBCB | 1155 | 36115502 | 611 | 36114891 | 36115501 | 13 |
| chr22 | UFD1 | 7353 | 19479184 | CDC45 | 8318 | 19479921 | 736 | 19479185 | 19479920 | 14 |
| chr2 | CCDC115 | 84317 | 130342152 | IMP4 | 92856 | 130342890 | 737 | 130342153 | 130342889 | 15 |
| chr3 | NAA50 | 80218 | 113746236 | ATP6V1A | 523 | 113747050 | 813 | 113746237 | 113747049 | 16 |
| chr12 | SART3 | 9733 | 108561169 | ISCU | 23479 | 108562603 | 1433 | 108561170 | 108562602 | 17 |
| chr1 | C1orf109 | 54955 | 37690515 | CDCA8 | 55143 | 37692536 | 2020 | 37690516 | 37692535 | 18 |
| chr10 | POLR3A | 11128 | 78029506 | RPS24 | 6229 | 78033862 | 4357 | 78029507 | 78033863 | 19 |
| chr19 | RPS16 | 6217 | 39435944 | SUPT5H | 6829 | 39445593 | 9648 | 39435945 | 39445592 | 20 |
| chr14 | RPS29 | 6235 | 49586376 | LRR1 | 122769 | 49598938 | 12561 | 49586377 | 49598937 | 21 |

* Between the TSS of gene 1 and the TSS of gene 2

According to a specific aspect, regulatory sequences selected from expression control sequences are employed, such as a promoter, an operator sequence, a transcriptional enhancer sequence or a transcriptional silencer sequence; or nucleotide sequences encoding expression factors, such as enhancers or inhibitors. Specifically, one or more regulatory sequences e.g., a promoter, are located between 2.000 or 1.000 bp upstream and 100 bp downstream of TSS of said GOI, in some cases of a small intergenic region, e.g., between any one of 1.000, 5.000, 1.000, 900, 800, 700, 600, 500, 400, 300, 200, 150, 100, or 50 bp upstream, and any one of 50 or 100 bp downstream a TSS.

Specifically, the transcription of the pair of the essential genes is in opposite orientation.

Specifically, each of the genes of a pair of essential genes is under transcriptional control of a respective essential gene promoter (EGP), preferably the EGP naturally-occurring with the respective gene, being either constitutive or inducible.

Specifically, the genes of said pair of essential genes are oriented such that their EGPs face in opposite directions.

Specifically, the EGPs are adjacent to each other.

More specifically, the EGPs are adjacent and face in opposite directions.

The respective promoter regions operably linked to the first and the second genes of a pair of essential genes are preferably adjacent, or in close proximity, such that the distance between the respective TSSs is less than any one of 20.000, 15.000, 10.000, 5.000, 2.500, 1.000, 900, 800, 700, 600, 500, or 400 nt.

Specifically, the intergenic region comprising the target site comprises or consists of the region between the respective TSSs. Preferably, the target site is comprised in such region between the TSSs where the two essential genes are encoded on opposite strands and the TSSs are in close proximity.

According to a specific embodiment, the target site is at least partly or fully comprised within a promoter region. For example, a transgene is inserted within a region that is considered a promoter of one of the two essential genes, in particular where it is proven that the essential gene function is unaffected by the insertion of the transgene.

Specifically, the intergenic region is the region between and spanning the transcriptional start sites of the EGPs.

Specifically, the essential genes are so called “core essential genes”, i.e., they are essential in many or all cell types or cell states studied to date. Consequently, their function needs to be maintained in all cell types and cell states of a certain species. For example, human core essential genes are considered essential in all human cell lines. Core essential genes may be determined by a genome-wide CRISPR/SpCas9 knockout screens, such as described by Hart el al. (G3: Genes, Genomes, Genetics Aug. 1, 2017 vol. 7 no. 8 2719-2727) or Wang et al. (Science. 2014; 343(6166):80-84). Exemplary human core essential genes are listed in Table 1 below (as gene 1 and/or gene 2).

According to a specific aspect, the GOI is under transcriptional control of a heterologous promoter. The GOI can be integrated at the target site in a forward or reverse orientation.

Specifically, the GOI is comprised in a heterologous expression cassette integrated at the target site. The expression cassette may comprise at least one of a control element, promoter or regulatory element, which is operatively linked to the GOI. In some embodiments, the control element, promoter or regulatory element operatively linked to the GOI is inducible.

Specifically, the GOI is a heterologous gene or a transgene.

Specifically, the GOI is selected from any one of: a nucleic acid, an inhibitor, a cDNA or gene encoding a peptide or polypeptide, and a cDNA or gene encoding an antibody or antibody fragment, fusion protein, antigen, antagonist, agonist, RNAi molecule, or miRNA. In some embodiments, the cDNA or gene encodes one or more transcription factors.

The target site is specifically understood as an insertion site, to insert the GOI or the expression cassette comprising the GOI.

The target site can comprise at least one or two sites (i.e., at least one or two locations) for the insertion of one or more GOIs (or expression constructs comprising a GOI) within the intergenic region at the target site. The intergenic region may be naturally-occurring or originating from a naturally occurring sequence and further be modified e.g., to incorporate a recognition target site or any other heterologous means to prepare for the knock-in of a transgene.

Besides insertion of the GOI (or the expression cassette comprising the GOI) at the target site, the intergenic region may be further modified by one or more mutations, such as deletion, insertion, or substitution of one or more nucleotides, which may be only a few e.g., up to 20 nt, or up to 15 nt, or up to 10 nt, but may also change the nucleotide sequence such that the modified intergenic region (excluding the GOI sequence or a heterologous expression cassette) has at least any one of 50%, 60%, 70%, 80%, 85%, 90%, 95%, 98%, or 99% sequence identity, or is 100% identical to the naturally occurring intergenic region.

The exemplary intergenic regions characterized by any one of SEQ ID NO:1-21 may or may not harbor one or more single-nucleotide polymorphisms, in particular those which are naturally-occurring. Specifically, the intergenic region is a naturally-occurring intergenic region which may differ from any one of SEQ ID NO:1-21 by one or more single-nucleotide polymorphisms, or which are identical to any one of SEQ ID NO:1-21 and devoid of any single-nucleotide polymorphisms.

Exemplary host cells are stem cells including e.g., a hematopoietic stem cell, an embryonic stem cell, a pluripotent stem cell, an induced pluripotent stem cell, or an endothelial cell.

According to a specific aspect, the host cell is a primary cell or an immune cell, such as of a cell type selected from the group consisting of a Natural Killer (NK) cell, a microglia cell, a macrophage, or a T cell, such as a cytotoxic T lymphocyte (CTL), a regulatory T cell or a T helper cell. Such host cell is specifically used in a pharmaceutical preparation, and/or cellular therapy and/or gene therapy. For example, engineered T cells may be engineered to express a T cell Receptor (TCR), specifically a heterologous TCR, or a chimeric antigen receptor (CAR).

Specifically, the host cell is an immune effector cell comprising a chimeric antigen receptor (CAR), wherein the GOI encodes the CAR expressed by said immune effector cell.

Specifically, the host cell is an immune effector cell comprising a chimeric antigen receptor (CAR), such as a CAR-T cell or a CAR macrophage, and the GOI comprises a nucleotide sequence encoding a CAR or part of a CAR, such as any one or more of the CAR domains, that can be expressed by said host cell on its surface.

According to a specific aspect, the host cell is of human origin, yet can be of non-human animal origin or mammalian, such as of mouse, rat, hamster, dog, pig or cattle. Specifically, the host cell is not a murine host cell.

According to another specific aspect, the host cell is a production host cell as used to produce a protein of interest in an in vitro host cell culture.

Specifically, the mammalian cell is a human or rodent or bovine cell, cell line or cell strain. Examples of specific mammalian cells suitable as host cells described herein are mouse myeloma (NSO)-cell lines, Chinese hamster ovary (CHO)-cell lines, HT1080, H9, HepG2, MCF7, MDBK Jurkat, MDCK, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EBI, EB2, EB3, oncolytic or hybridoma-cell lines.

The invention further provides for an isolated clone of the host cell described herein, or an in vitro cell culture of such clone.

According to a specific aspect, the invention provides for a pharmaceutical composition comprising the host cell or a population of host cells described herein, and a pharmaceutically acceptable carrier.

The invention further provides for a preparation comprising a population of host cells, in particular a population or repertoire of different (e.g., polyclonal) cells, wherein at least any one of 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of the cells are host cells described herein, with chromosomal identity, or with the identical genotype or identical chromosomotype (i.e., identity based on chromosomal evidence).

Specific examples refer to pharmaceutical preparation comprising a population of CAR-T cells, wherein at least any one of 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of the cells are host cells described herein, with chromosomal identity or with the identical genotype or identical chromosomotype, which population stably expresses the GOI, such as to provide for expression over a prolonged period of time.

The invention further provides for an in vitro host cell culture of a clone or the preparation described herein, which is maintained under conditions to express said GOI, in particular to stably express said GOI, thereby either obtaining the respective polypeptide or protein of interest e.g., which is bound on the surface of the host cell, or secreted into the host cell culture medium.

The invention further provides for a non-human animal comprising the host cell described herein. Specifically, the non-human animal is a genetically modified animal (also referred to as a transgenic animal) comprising a donor sequence comprising the GOI, which donor sequence is inserted at a predetermined insertion site on a chromosome of the animal, wherein the predetermined insertion site is the target site further described herein. Specifically, the methods described herein do not comprise modifying the germ line genetic identity of human beings.

An exemplary production method comprises,
a) generating a cell with the donor sequence inserted at the predetermined insertion site; and
b) introducing the cell generated by a) into a carrier animal to produce the genetically modified animal.

Specifically, the cell is a zygote or a pluripotent stem cell.

The invention further provides for a wildcard mammalian host cell comprising heterologous means for site-directed chromosomal integration of an exogenous gene of interest (GOI), in particular a heterologous GOI, at a target site within an intergenic region between a pair of adjacent essential genes. Such wildcard host cell is preferably a diploid host cell.

Exemplary means may be one or more nucleic acid modifications to incorporate a restriction cloning site into the intergenic region, or to incorporate one or more target sites for one or more nucleases. The respective nucleases may be selected from a zinc finger nuclease (ZFN), a TAL-effector domain nuclease (TALEN), or a CRISPR/Cas system. A specific target site may be for a guide RNA (gRNA), such as for a sequence-specific nuclease selected from any of: a TAL-nuclease, a zinc-finger nuclease (ZFN), a meganuclease, a megaTAL, or an RNA guide endonuclease (e.g., Cas9, Cpf1, Cas9 nickase).

Specific means may include one or more nucleic acid modifications to the naturally-occurring intergenic sequence at or adjacent to the target site.

According to a specific aspect, the means are selected from the group consisting of heterologous target sites recognized by a recombinase to mediate the knock-in of a transgene at the respective target site. For example, recognition target sites can be incorporated within the intergenic region as described herein to engineer the target site, and the respective recombinase recognizing such target sites is used to chromosomally integrate the GOI. Any of the commonly used site-directed recombination technology can be used, e.g., those requiring preparatory means to provide the wildcard host cell as described herein. Exemplary site-directed recombination technologies are Flp-FRT recombination, Cre-Lox recombination, or phage lambda site-specific recombination

The invention further provides for a production method to produce recombinant host cells expressing a heterologous GOI, wherein the GOI is inserted at a target site comprised in an intergenic region between a pair of adjacent essential genes.

The invention further provides for a method of producing a gene product encoded by a gene of interest (GOI), by transforming a host cell to chromosomally integrate the GOI within an intergenic region between a pair of adjacent essential genes, and culturing the transformed host cell under conditions to express said GOI, preferably wherein each of the adjacent essential genes of said pair has a transcription start site (TSS) and the distance between the TSSs is least any one of 80, 90, 100, or 110 nt, and less than any one of 20.000, 15.000, 10.000, 5.000, 2.500, or 1.000 nt.

The invention further provides for a method of modifying a mammalian cell by site directed chromosomal integration of an exogenous, in particular a heterologous GOI, gene of interest (GOI), in particular a heterologous GOI, within an intergenic region between a pair of adjacent essential genes, preferably wherein each of the adjacent essential genes of said pair has a transcription start site (TSS) and the distance between the TSSs is least any one of 80, 90, 100, or 110 nt, and less than any one of 20.000, 15.000, 10.000, 5.000, 2.500, or 1.000 nt.

According to a specific embodiment, the GOI (or the expression cassette comprising the GOI) is inserted by a method employing a nuclease. The nuclease may comprise a DNA binding domain to bind to the target site, and a nuclease cleavage domain. In particular a nuclease is used to generate a double strand break (DSB) at the target site or within the intergenic region. The double-strand break could facilitate insertion of the transgene by non-homologous end-joining or, in the presence of a donor sequences bearing homologous regions, by homology-directed repair.

Specifically, the method employs any one of a programmable nuclease, such as an engineered endonuclease, preferably a zinc finger nuclease (ZFN), a transcription activator-like effector nuclease (TALEN), a CRISPR endonuclease or an Argonaute protein for chromosomal integration. In particular, the method employs a gene-editing protein selected from a CRISPR/Cas9, TALEN and a zinc finger nuclease.

Specifically, the method employs a gene editing nucleic acid sequence. The gene editing nucleic acid sequence may encode a gene editing molecule selected from the group consisting of: a sequence specific nuclease, one or more guide RNA, CRISPR/Cas, MAD7, a ribonucleoprotein (RNP), or deactivated CAS for CRISPRi or CRISPRa systems, or any combination thereof. The endonuclease activity of a protein can be assessed by techniques known to those of skill in the art.

Specific preferred host cells are those transduced with the CRISPR-Cas system.

Argonaute family proteins may as well be used, as well-known in the art. For example, TtAgo, PfAgo and NgAgo have been shown to elicit targeted DNA double-strand breaks that could be used to facilitate transgene insertion by homology-direct repair (Swarts et al. Nature. 2014;507(7491):258-261).

Specifically, the method employs means or method steps of a genome editing technique to insert the GOI at the target site.

According to a specific aspect, the intergenic region is modified to insert, at least one or more of the following:
(i) a gene editing nucleic acid sequence;
(ii) a target site for one or more nucleases;
(iii) a GOI; or
(iv) a guide RNA (gRNA) recognition site for an RNA-guided DNA endonuclease.

According to a specific embodiment, the GOI is inserted into the cellular genome by homologous recombination. For example, a donor sequence comprising the GOI may be inserted into the chromosome at or adjacent to the target site through homologous recombination. Specifically, a vector for gene editing can be used for knock-in of a desired nucleic acid sequence.

To this end, a vector is conveniently used e.g., a viral vector, such as an adeno-associated viral vector, which comprises a target site 5’ homology arm, the expression cassette comprising the GOI, and a target site 3’ homology arm, wherein the 5’ homology arm and the 3′ homology arm bind to the target site located in the intergenic region, in particular within the intergenic region, and wherein the 5’ and 3’ homology arms guide homologous recombination into a locus (in particular a safe harbor locus) located within the intergenic region. Exemplary 5’ and 3’ homology arms are between 30-2000 bp in length. The 5’ homology arm and the 3’ homology arm particularly bind to target sites that are spatially distinct nucleic acid sequences in the same intergenic region. A certain degree of homology is typically employed e.g., any one or both of the homology arms can be at least any one of 65%, 70%, 75%, 80%, 85%, 90%, 91%, 92%, 93%, 94%, 95%, 96%, 97%, 98%, 99%, or 100% homology complementary to a target sequence within the intergenic region to hybridize with such complementary target sequence, or be 100% homologous and complementary to the target sequence.

Specific methods of homologous recombination described herein may employ a vector comprising:
a) an expression cassette comprising a promoter and at least one GOI, or comprising a promoter operably linked to at least one GOI; and
b) two self-complementary sequences, e.g., asymmetrical or symmetrical, flanking said expression cassette, which are a 5’ homology arm being homologous to a nucleotide sequence upstream of a nuclease cleavage site, and a 3’ homology arm being homologous to a nucleotide sequence downstream of the nuclease cleavage site.

The invention further provides for a set of tools for genetically engineering a host cell, comprising
a) a wildcard host cell described herein; and
b) a vector and optionally auxiliary agents adapted to said means for site-directed chromosomal integration of an expression cassette that expresses the GOI upon site directed integration.

Specifically, the GOI is supplied by or comprised in an expression cassette that is incorporated at the target site. The expression cassette may comprise one or more regulatory elements, in particular a promoter, and at least one GOI. Specifically, the expression cassette is a heterologous one.

The invention further provides for a substance, composition of matter or material, which is selected from the host cell, the clone, the preparation, the cell culture, the wildcard host cell, and the set of tools described herein, for medical use, in particular for cellular therapy (e.g., employing CAR-T cells or CAR macrophage cells), T cell receptors (for TCR therapy), or cDNAs for gene therapy.

A specific application of the gene therapy involves the treatment of disorders that are either caused by an insufficiency of a secreted gene product or that are treatable by secretion of a therapeutic protein. Such disorders are potentially addressable via delivery of a therapeutic GOI to a number of cells, provided that each recipient cell expresses a high level of the therapeutic GOI. Such applications typically require stable, safe, and high levels of transgene expression.

A therapeutic GOI may be a cDNA which when chromosomally integrated into the target cell is expressed for therapeutic activity, in particular for gene therapy.

Exemplary methods of gene therapy employ a therapeutic GOI expressing a coagulation factor e.g., factor VIII or factor IX, for use in a method of treating hemophilia, such as hemophilia A or B.

Specifically, the substance, composition of matter or material is used as described herein wherein a subject is treated by expressing said GOI in vivo, e.g., by a transgenic animal, such as a human being or a non-human animal. Typically, the in vivo use is for gene therapy.

The invention further provides for a method of treating a subject comprising administering an effective amount of a substance, composition of matter or material, which is selected from the host cell, the clone, the preparation, the cell culture, the wildcard host cell, and the set of tools described herein, to express said GOI in vivo.

According to a specific aspect, the medical use comprises treating a cancer patient by a method comprising:
a) isolating a cell from a mammalian subject, preferably autologous or allogenic to the cancer patient, the cell being an immune cell or hematopoietic cell;
b) modifying the cell by one or more genetic modifications to chromosomally integrate a heterologous gene of interest (GOI) at a target site within an intergenic region between a pair of adjacent essential genes, wherein the GOI encodes a chimeric antigen receptor (CAR); and
c) administering the cell to the subject.

Specifically, the immune cell is a T cell, a microglia, or a macrophage.

The invention further provides for a method for making a chimeric antigen receptor (CAR) T cell, comprising:
(a) obtaining a T cell from a mammalian subject, preferably a human T cell; and
(b) modifying the T cell by one or more genetic modifications to chromosomally integrate a heterologous gene of interest (GOI) at a target site within an intergenic region between a pair of adjacent essential genes, wherein the GOI encodes a chimeric antigen receptor (CAR).

The invention further provides for a method for making or producing a chimeric antigen receptor (CAR) microglia or macrophage, comprising:
(a) obtaining a microglia or macrophage from a mammalian subject, preferably a human microglia or macrophage; and
(b) modifying the microglia or macrophage by one or more genetic modifications to chromosomally integrate a heterologous gene of interest (GOI) at a target site within an intergenic region between a pair of adjacent essential genes, wherein the GOI encodes a chimeric antigen receptor (CAR).

Specifically, the modified microglia or macrophage is characterized by features of the host cell as further described herein.

Specifically, the intergenic region between the pair of adjacent essential genes is characterized as further described herein, such as characterized by one or more of he following preferred features:
a) the intergenic region is between a pair of adjacent essential genes, wherein each of the adjacent essential genes of said pair has a transcription start site (TSS) and the distance between the TSSs is less than 20.000 nt, and preferably at least 80 nt;
b) the length of the intergenic region is less than 20.000 nt;
c) the intergenic region comprises or consists of the nucleic acid sequence which is at least 90% identical to any one of SEQ ID NO:1-21;
d) the essential genes of said pair are oriented such that their promoters face in opposite directions; or
e) the GOI is comprised in a heterologous expression cassette integrated at the target site.The invention further provides for an ex vivo use of the substance, composition of matter or material, which is selected from the host cell, the clone, the preparation, the cell culture, the wildcard host cell, and the set of tools described herein, for in vitro engineering transgenic cells or recombinant host cells. Such cells can be provided for expressing the GOI in vitro e.g., in a cell culture, or expressing the GOI in vivo e.g., upon administering the transgenic cells to a subject in need of such cellular treatment.

### FIGURES

FIG. 1: Transgene expression at the indicated locus is not silenced. Transgenes are inserted at a locus that resides between two essential genes, where the essential gene promoters are located in close proximity. Epigenetic silencing of the transgene spreads to one of the neighbouring essential genes (or both). This will lead to cell death. Consequently, all surviving cells maintain transgene expression at high level.

FIG. 2: Experimental approach to fine-map the region that allows transgene insertion. Transgene insertion may compromise cell viability. To map the precise location where transgene insertion is tolerated, the region between two essential genes is tiled with an array of guide RNAs. As reference, the neighboring essential genes are also targeted with a suitable set of guide RNAs. Guide RNAs whose activity is tolerated by the cell are identified in the ensuing CRISPR screen.

FIG. 3: Experimental approach to assess the impact of silencing at the specified locus. A locus between two essential genes is targeted with an expression cassette harboring a reporter gene (eGFP) whose activity is regulated by a suitable constitutive promoter (SFFV). Nearby, an array of TetO sites is included. Upon insertion of the transgene, cells turn GFP-positive as transgene expression is supported. A population of GFP-positive cells is then subjected to treatment with TetR-KRAB. TetR-KRAB silences the TetO sites, which leads to dampened GFP expression. As silencing spreads to the neighboring essential genes, cells in which GFP silencing has occurred succumb to cell death. GFP-negative survivors are not isolated.

FIG. 4: CRISPR screen to identify feasible loci for intergenic targeting. In this screen, essential genes, intergenic regions and non-essential genes were targeted with a set of sgRNAs (as described in the Examples). Cells were harvested at day 3 and day 21, genomic DNA was extracted and guide RNAs were amplified by PCR. In this figure, the fold change between day 21 and day 3 was plotted for each sgRNA queried here.

FIG. 5: CRISPR screen to identify feasible loci for intergenic targeting. In this screen, essential genes, intergenic regions and non-essential genes were targeted with a set of sgRNAs (as described in the Examples). Cells were harvested at day 3 and day 21, genomic DNA was extracted and guide RNAs were amplified by PCR. In this figure, the fold change between day 21 and day 3 was aggregated at the level of each gene/ locus and was then plotted.

FIG. 6: Schematic of transgene insertion and PCR verification at the different loci. Three loci (AAVS1, MED, FTSJ3) were targeted as described in the Examples. Presence of the transgene cassette (SV40-mKate2-polyA) was verified by PCR as indicated.

FIG. 7: Confirmation of transgene expression by PCR. Genomic DNA was isolated from cells harboring a transgene insertion between two essential genes (see Examples for details). Next, PCRs were conducted in which one primer targets the transgene cassette and the other targets the region outside the homology arms (see FIG. 6 for a schematic).

FIG. 8: Transgene expression is detected by flow cytometry. Cells harboring the SV40-mKate2-polyA transgene at two loci (MED20, FTSJ3) were analyzed by flowcytometry. mKate2 expression was monitored in the PE channel.

FIG. 9: Sequences as herein described.

SEQ ID NO:1-21 : human intergenic regions

SEQ ID NO:26:
MED20 Donor1 (TetO sites in bold, eGFP underlined, 2A-PuroR in italic).

SEQ ID NO:27:
MED20 Donor 2 (TetO sites in bold, eGFP underlined, 2A-PuroR in italic).

SEQ ID NO:28:
FTSJ3 Donor 1 (TetO sites in bold, eGFP underlined, 2A-PuroR in italic).

SEQ ID NO:29:
FTSJ3 Donor 2 (TetO sites in bold, eGFP underlined, 2A-PuroR in italic).

SEQ ID NO:30:
TetR-KRAB

SEQ ID NO:35:
MED20 homology donor (Left homology arm bold, right homology arm italic)

SEQ ID NO:36:
FTSJ3 homology donor (Left homology arm in bold, right homology arm in italic)

SEQ ID NO:37:
Cas9 expression vector (with AAVS1 sgRNA integrated)

SEQ ID NO:38:
AAVS1 homology donor (Left homology arm in bold, right homology arm in italic,
transgene cassette in capital letters, SV40 promoter in underscore, mKate2 in bold and
underscore, polyadenylation signal in bold and italic).

SEQ ID NO:39 (MED20 sgRNA): GGGCGTGTCTCGGCACCCCT

SEQ IDNO:40 (FTSJ3 sgRNA): GAATTCCGGGTCAATGGGCG

SEQ IDNO:41 (AAVS1 sgRNA): gtccctagtggccccactgtg

### DETAILED DESCRIPTION OF THE INVENTION

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (2nd Ed.), Vols. 1 -3, Cold Spring Harbor Laboratory Press (1989); Lewin, “Genes IV”, Oxford University Press, New York, (1990), and Janeway et al, “Immunobiology” (5th Ed., or more recent editions), Garland Science, New York, 2001.

The subject matter of the claims specifically refers to artificial products or methods employing or producing such artificial products, which may be variants of native (wild-type) products. Though there can be a certain degree of sequence identity to the native structure, it is well understood that the materials, methods and uses of the invention, e.g., specifically referring to isolated nucleic acid sequences, amino acid sequences, fusion constructs, expression constructs, transformed host cells and modified proteins, are “man-made” or synthetic, and are therefore not considered as a result of “laws of nature”.

Specific terms as used throughout the specification have the following meaning.

The term “host cell” as used herein is understood as any cell type that is susceptible to transformation, transfection, transduction, or the like with nucleic acid constructs or expression vectors comprising one or more polynucleotides encoding expression products described herein, or susceptible to otherwise introduce any or each of the components of the CRISPR complex described herein. The term “host cell” encompasses any progeny of a parent cell that is not identical to the parent cell due to modifications e.g., by a method described herein, or that occur during replication, and shall particularly refer to a single cell, a single cell clone, a population of cells, such as a population comprising polyclonal cells, or a cell line of a host cell.

The term “cell line” as used herein refers to an established clone of a particular cell type that has acquired the ability to proliferate over a prolonged period of time. A cell line is typically used for expressing an endogenous or recombinant gene, or products of a metabolic pathway to produce polypeptides or cell metabolites mediated by such polypeptides. A “production host cell line” or “production cell line”, such as used herein for producing a POI, is commonly understood to be a cell line ready-to-use for cell culture in a bioreactor to obtain the product of a production process. A recombinant POI can be produced using the host cell and the respective cell line described herein, by culturing in an appropriate medium, isolating the expressed product or metabolite from the culture, and optionally purifying it by a suitable method.

As used herein, the term “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as a product of gene expression (“gene product”), such as produced by expressing a GOI. If the polynucleotide is derived from genomic DNA, expression may include splicing of the mRNA in a eukaryotic cell.

The term “expression cassette” as used herein refers to a nucleotide sequence or respective polynucleotide or nucleic acid molecule containing a desired coding sequence and regulatory or control sequences in operable linkage, so that hosts transformed or transfected with these sequences are capable of producing the encoded proteins or host cell metabolites. Exemplary expression control sequences may include any of a promoter, ribosomal binding site, transcriptional or translational start and stop sequences, or of an enhancer or activator sequence.

An expression cassette may comprise at least one intron. Usually, introns are placed at the 5’ end of the open reading frame but may also be placed at the 3’ end. Said intron may be located between the promoter and or promoter/enhancer element(s) and the 5’ end of the open reading frame of the polynucleotide encoding the product of interest to be expressed. Several suitable introns are known in the state of the art that can be used in conjunction with the present disclosure.

In order to effect transformation, the expression system may be included in an expression construct e.g., in the form of a “vector”, or expression cassettes integrated in a host cell’s chromosome. Expression cassettes are typically DNA sequences that are required for the transcription of cloned recombinant nucleotide sequences, i.e. of recombinant genes and the translation of their mRNA in a suitable host organism. Expression vectors usually comprise one or more of an origin for autonomous replication or a locus for genome integration in the host cells, selectable markers (e.g., an amino acid synthesis gene or a gene conferring resistance to antibiotics such as zeocin, kanamycin, G418 or hygromycin, nourseothricin), a number of restriction enzyme cleavage sites, and regulatory sequences e.g., any one or more of a suitable promoter sequence, operator, enhancer, ribosomal binding site, and sequences that control transcription and translation initiation and termination. The regulatory sequences are typically operably linked to the DNA sequence to be expressed.

A “vector” is herein understood to be capable of transferring nucleic acid sequences to target cells (e.g., viral vectors, non-viral vectors, particulate carriers, and liposomes). Typically, the vector is understood as an “expression vector” or a “gene transfer vector”, which is any nucleic acid construct capable of directing the expression of a nucleic acid of interest and which can transfer nucleic acid sequences to target cells. Thus, the term includes cloning and expression vehicles, as well as viral vectors.

Specific vectors include autonomously replicating nucleotide sequences (e.g. plasmids) as well as genome integrating nucleotide sequences, such as artificial chromosomes e.g., a yeast artificial chromosome (YAC). Expression vectors may include but are not limited to cloning vectors, modified cloning vectors and specifically designed plasmids. Preferred expression vectors described herein are expression vectors suitable for expressing of a recombinant gene in a eukaryotic host cell and are selected depending on the host organism.

The relevant DNA may be integrated into a host cell chromosome. Expression by a host cell may refer to secreted or non-secreted expression products, including polypeptides or metabolites. To allow expression of a recombinant nucleotide sequence in a host cell, the expression cassette or vector described herein typically comprises a promoter operably linked to the GOI, e.g., a promoter nucleotide sequence which is adjacent to the 5’ end of the coding sequence, or upstream from and adjacent to a gene of interest (GOI), or if a signal or leader sequence is used, upstream from and adjacent to said signal and leader sequence, respectively, to facilitate expression of the GOI. The promoter sequence is typically regulating and initiating transcription of the downstream nucleotide sequence, with which it is operably linked, including in particular the GOI.

Specifically, the promoter is a heterologous promoter, in particular heterologous to the host cell and/or not natively associated with the GOI.

In specific embodiments, multicloning vectors may be used, which are vectors having a multicloning site. Specifically, a desired heterologous gene can be integrated or incorporated at a multicloning site to prepare an expression vector. In the case of multicloning vectors, a promoter is typically placed upstream of the multicloning site.

Specifically, preferred embodiments of a CRISPR system employ a delivery system, comprising one or more vectors, optionally wherein the vectors comprise one or more viral vectors. Specifically, one or more viral vectors may be used which are selected from the group consisting of lentivirus, retrovirus, adenovirus, adeno-associated virus or herpes simplex virus, lentiviral, adenoviral or adeno-associated viral (AAV) vectors. Specific examples are selected from the group consisting of HIV-based lentiviruses. Lentiviral vectors may harbor certain safety features, e.g., they may rely on multiple packaging plasmids or they may have truncated long terminal repeats. All of these features are deemed to reduce the chance of obtaining a replication-competent virus, i.e., typically, these viruses can only undergo a single infection cycle.

In some alternative embodiments, elements of a CRISPR system are delivered via liposomes, particles, cell penetrating peptides, exosomes, microvesicles, or a gene-gun or via electroporation of the target cell.

The expression level can be determined quantitatively or qualitatively, by measuring the mRNA or protein level of said at least one gene, in particular the level of an expression product of a target gene. Specifically, the expression level can be determined by measuring the host cell’s transcriptome, or by assessing protein levels at the cell surface by flow cytometry.

In certain embodiments, the expression level is determined using a method selected from the group consisting of quantitative RT-PCR (qPCR), RNA sequencing, hybridization to a microarray, and molecular tagging.

“Gene expression” specifically refers to the conversion of the information, contained in a gene, into a gene product. Gene expression is meant to encompass at least one step selected from the group consisting of DNA transcription into mRNA, mRNA processing, mRNA maturation, mRNA export, translation, protein folding and/or protein transport.

As used herein, the term “gene” e.g., as used in the term “gene of interest” or “GOI” includes a DNA region encoding a gene product, and optionally one or more (or all) DNA regions which regulate the production of the gene product, whether or not such regulatory sequences are adjacent to coding and/or transcribed sequences. Accordingly, a gene may be understood to include promoter sequences, terminators, translational regulatory sequences such as ribosome binding sites and internal ribosome entry sites, enhancers, silencers, insulators, boundary elements, replication origins, matrix attachment sites and locus control regions.

According to further specific examples, a GOI as used in the context of the present invention is a cDNA, and/or encodes a biological product, a ligand (such as an antibody), or a receptor, such as chimeric antigen receptor (CAR) or a T cell receptor.

A suitable CAR may include an antigen-binding part or domain (as an extracellular binding portion), a transmembrane domain, and an intracellular signaling domain, and optionally further includes one or more linker sequences between the various domains.

Specifically, the CAR includes an antigen-binding portion that binds to a target antigen of interest, e.g., a particular antigen on the surface of a target cell. For example, the antigen-binding portion that may be comprised in the CAR may include an antibody, a receptor (e.g., a variable T cell receptor or lymphocyte receptor), a receptor fragment (e.g., an Fc receptor fragment), a ligand, a cytokine, a DARPin, an adnectin, a nanobody, and a peptide.

The antibody included in the antigen-binding portion is preferably capable of specifically recognizing an antigen of clinical relevance and may be selected from the group consisting of a full-length antibody, and antibody fragment comprising at least one antibody variable domain or antigen-binding site (preferably a single-chain variable fragment, scFv).

The transmembrane domain specifically fuses the extracellular binding portion and intracellular signaling domain and anchors the CAR to the plasma membrane of the cell. Various suitable transmembrane domains are known in the art.

According to a specific aspect, at least one intracellular signaling domain of a CAR is used.

The intracellular signaling domain of a CAR specifically is the part of a CAR that participates in transducing the signal from CAR binding to a target cell into the interior of the immune effector cell to elicit effector cell function, e.g., activation, cytokine production, proliferation and/or cytotoxic activity, including the release of cytotoxic factors to the CAR-bound target cell, or other cellular responses elicited with target binding to the extracellular CAR domain.

The intracellular signaling domain of the CAR is responsible for activation of at least one of the normal effector functions of the immune cell in which the CAR has been placed in. Effector function of a T cell, for example, may be cytolytic activity or helper activity including the secretion of cytokines. Specifically, the term “intracellular signaling domain” refers to the portion of a protein which transduces the effector function signal and directs the cell to perform a specialized function.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

T cell activation can be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the CARs disclosed herein include those derived from TCR zeta (CD3 Zeta), FcR gamma, FcR beta, CD3 gamma, CD3 delta, CD3 epsilon, CD5, CD22, CD79a, CD79b, and CD66d. Specific, non-limiting examples, of the ITAM include peptides having sequences of amino acid numbers 51 to 164 of CD3.zeta. (NCBI RefSeq: NP.sub.--932170.1), amino acid numbers 45 to 86 of Fc.epsilon.Rl.gamma. (NCBI RefSeq: NP.sub.--004097.1), amino acid numbers 201 to 244 of Fc.epsilon.Rl.beta. (NCBI RefSeq: NP.sub.--000130.1), amino acid numbers 139 to 182 of CD3.gamma. (NCBI RefSeq: NP.sub.--000064.1), amino acid numbers 128 to 171 of CD3.delta. (NCBI RefSeq: NP.sub.--000723.1), amino acid numbers 153 to 207 of CD3.epsilon. (NCBI RefSeq: NP.sub.--000724.1), amino acid numbers 402 to 495 of CD5 (NCBI RefSeq: NP.sub.--055022.2), amino acid numbers 707 to 847 of 0022 (NCBI RefSeq: NP.sub.--001762.2), amino acid numbers 166 to 226 of CD79a (NCBI RefSeq: NP.sub.--001774.1), amino acid numbers 182 to 229 of CD79b (NCBI RefSeq: NP.sub.-000617.1), and amino acid numbers 177 to 252 of CD66d (NCBI RefSeq: NP.sub.--001806.2), and their variants having the same function as these peptides have. The amino acid number based on amino acid sequence information of NCBI RefSeq ID or GenBank described herein is numbered based on the full length of the precursor (comprising a signal peptide sequence etc.) of each protein.

In one embodiment, the cytoplasmic signaling molecule in the CAR comprises a cytoplasmic signaling sequence derived from CD3 zeta.

In a specific embodiment, the intracellular domain of the CAR can be designed to comprise the CD3-zeta signaling domain by itself or combined with any other desired cytoplasmic domain(s) useful in the context of the CAR. For example, the intracellular domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such costimulatory molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

Specific, non-limiting examples, of such costimulatory molecules include peptides having sequences of amino acid numbers 236 to 351 of CD2 (NCBI RefSeq: NP.sub.--001758.2), amino acid numbers 421 to 458 of CD4 (NCBI RefSeq: NP.sub.--000607.1), amino acid numbers 402 to 495 of CD5 (NCBI RefSeq: NP.sub.--055022.2), amino acid numbers 207 to 235 of CD8.alpha. (NCBI RefSeq: NP.sub.--001759.3), amino acid numbers 196 to 210 of CD83 (GenBank: AAA35664.1), amino acid numbers 181 to 220 of CD28 (NCBI RefSeq: NP.sub.--006130.1), amino acid numbers 214 to 255 of CD137 (4-1BB, NCBI RefSeq: NP.sub.--001552.2), amino acid numbers 241 to 277 of CD134 (OX40, NCBI RefSeq: NP.sub.--003318.1), and amino acid numbers 166 to 199 of ICOS (NCBI RefSeq: NP.sub.--036224.1), and their variants having the same function as these peptides have. Thus, while the disclosure herein is exemplified primarily with 4-1 BB as the co-stimulatory signaling element, other costimulatory elements are within the scope of the disclosure.

The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

In a specific embodiment, the intracellular domain is designed to comprise the signaling domain of CD3-zeta and the signaling domain of CD28. In another embodiment, the intracellular domain is designed to comprise the signaling domain of CD3-zeta and the signaling domain of 4-1BB. In yet another embodiment, the intracellular domain is designed to comprise the signaling domain of CD3-zeta and the signaling domain of CD28 and 4-1BB.

Specific preferred embodiments include any one or more of the following:
a) the intracellular signaling domain comprises a CD3 zeta intracellular domain,
b) the intracellular signaling domain comprises a costimulatory domain, a primary signaling domain, or any combination thereof,
c) the intracellular signaling domain comprises a costimulatory domain comprising a functional signaling domain of a protein selected from the group consisting of: OX40, CD70, CD27, CD28, CD5, ICAM-1, LFA-1 (CD11a/CD18), ICOS (CD278), DAP10, DAP12, and 4-1 BB (CD137).

According to a specific aspect, the CAR specifically recognizes and binds to a molecule on the surface of the target cell. According to specific examples, the target cell is a tumor or cancer cell, and the CAR specifically recognizes a tumor associated antigen (TAA), preferably any one of CD19, CD20, CD22, B7-H3 (CD276), CD133, GD2, EGFRvlll, BMSA, MSLN, CEA and HER2.

According to further specific examples, a GOI as used in the context of the present invention is selected from the group consisting of DNA vaccines, or therapeutic DNA molecules e.g., a therapeutic DNA molecule which
a) expresses a functional gene in a subject having a genetic disorder caused by a dysfunctional version of said functional gene (e.g., gene for Duchenne muscular dystrophy, cystic fibrosis, Gaucher’s Disease, and adenosine deaminase (ADA) deficiency, inflammatory diseases, autoimmune, chronic and infectious diseases, AIDS, cancer, neurological diseases, cardiovascular disease, hypercholesterolemia, various blood disorders (including various anaemias, thalassemia and haemophilia, and emphysema), and solid tumors);
b) encodes a toxic peptide (i.e., chemotherapeutic agents such as ricin, diphtheria toxin and cobra venom factor), tumor suppressor genes (such as p53), genes coding for mRNA sequences which are antisense to transforming oncogenes, antineoplastic peptides such as tumor necrosis factor (TNF) and other cytokines, or transdominant negative mutants of transforming oncogenes;
c) encodes an active RNA form (e.g., a small interfering RNA (siRNA, miRNA, shRNA), or a small activating RNA (saRNA); or,
d) encodes a CRISPR/Cas component (such as a Cas9 enzyme or an sgRNA).
e) According to further specific examples, the GOI as used in the context of the present invention expresses a protein of interest (POI) e.g., in a cell culture.

Specifically, the POI is heterologous to the host cell species.

Specifically, the POI is a secreted peptide, polypeptide, or protein, i.e., secreted from the host cell into the cell culture supernatant.

Specifically, the POI is a eukaryotic protein, preferably a mammalian derived or related protein such as a human protein or a protein comprising a human protein sequence, or a bacterial protein or bacterial derived protein

Preferably, the POI is a therapeutic protein functioning in mammals.

In specific cases, the POI is a multimeric protein, specifically a dimer or tetramer.

According to a specific aspect, the POI is a peptide or protein selected from the group consisting of an antigen-binding protein, a therapeutic protein, an enzyme, a peptide, a protein antibiotic, a toxin fusion protein, a carbohydrate - protein conjugate, a structural protein, a regulatory protein, one or more transcription factors, a vaccine antigen, a growth factor, a hormone, a cytokine, a process enzyme, and a metabolic enzyme.

Specifically, the antigen-binding protein is selected from the group consisting of
a) antibodies or antibody fragments, such as any of chimeric antibodies, humanized antibodies, bi-specific antibodies, Fab, Fd, scFv, diabodies, triabodies, Fv tetramers, minibodies, single-domain antibodies like VH, VHH, IgNARs, or V-NAR;
b) antibody mimetics, such as Adnectins, Affibodies, Affilins, Affimers, Affitins, Alphabodies, Anticalins, Avimers, DARPins, Fynomers, Kunitz domain peptides, Monobodies, or NanoCLAMPS; or
c) fusion proteins comprising one or more immunoglobulin-fold domains, antibody domains or antibody mimetics.

A specific POI is an antigen-binding molecule such as an antibody, or a fragment thereof, in particular an antibody fragment comprising an antigen-binding domain. Among specific POIs are antibodies such as monoclonal antibodies (mAbs), immunoglobulin (Ig) or immunoglobulin class G (IgG), heavy-chain antibodies (HcAb’s), or fragments thereof such as fragment-antigen binding (Fab), Fd, single-chain variable fragment (scFv), or engineered variants thereof such as for example Fv dimers (diabodies), Fv trimers (triabodies), Fv tetramers, or minibodies and single-domain antibodies like VH, VHH, IgNARs, or V-NAR, or any protein comprising an immunoglobulin-fold domain. Further antigen-binding molecules may be selected from antibody mimetics, or (alternative) scaffold proteins such as e.g., engineered Kunitz domains, Adnectins, Affibodies, Affiline, Anticalins, or DARPins.

The term “endogenous” as used herein is meant to include those molecules and sequences, in particular endogenous genes or proteins, which are present in a naturally-occurring, wild-type (native) host cell. In particular, an endogenous nucleic acid molecule (e.g., a gene) or protein that does occur in (and can be obtained from) a particular host cell as it is found in nature, is understood to be “host cell endogenous” or “endogenous to the host cell”. Moreover, a cell “endogenously expressing” a nucleic acid or protein expresses that nucleic acid or protein as does a host of the same particular type as it is found in nature. Moreover, a host cell “endogenously producing” or that “endogenously produces” a nucleic acid, protein, or other compound produces that nucleic acid, protein, or compound as does a host cell of the same particular type as it is found in nature.

The term “heterologous” as used herein with respect to a nucleotide sequence, construct such as GOI, a promoter, an expression cassette, amino acid sequence or protein, refers to a compound which is either foreign to a given host cell, i.e., “exogenous”, such as not found in nature in said host cell; or that is naturally found in a given host cell, e.g., is “endogenous”, however, in the context of a heterologous construct or integrated in such heterologous construct, e.g., employing a heterologous nucleic acid fused or in conjunction with an endogenous nucleic acid, thereby rendering the construct heterologous. The heterologous nucleotide sequence as found endogenously may also be produced in an unnatural, e.g., greater than expected or greater than naturally found, amount in the cell. The heterologous nucleotide sequence, or a nucleic acid comprising the heterologous nucleotide sequence, possibly differs in sequence from the endogenous nucleotide sequence but encodes the same protein as found endogenously. Specifically, heterologous nucleotide sequences are those not found in the same relationship to a host cell in nature. Any recombinant or artificial nucleotide sequence is understood to be heterologous. An example of a heterologous polynucleotide is a nucleotide sequence not natively associated with a promoter, e.g., to obtain a hybrid promoter, or operably linked to a coding sequence, as described herein. As a result, a hybrid or chimeric polynucleotide may be obtained. A further example of a heterologous compound is a POI encoding polynucleotide operably linked to a transcriptional control element, e.g., a promoter, to which an endogenous, naturally-occurring POI coding sequence is not normally operably linked.

The term “isolated” or “isolation” as used herein shall refer to such compound that has been sufficiently separated from the environment with which it would naturally be associated, so as to exist in “purified” or “substantially pure” form. The term “isolated” can refer to material that is free, substantially free, or essentially free to varying degrees from components which normally accompany it as found in its native state. “Isolate” also denotes a degree of separation from original source or surroundings. Yet, “isolated” does not necessarily mean the exclusion of artificial or synthetic mixtures with other compounds or materials, or the presence of impurities that do not interfere with the fundamental activity, and that may be present, for example, due to incomplete purification. Isolated compounds can be further formulated to produce preparations thereof, and still for practical purposes be isolated - for example, host cells or a POI can be mixed with pharmaceutically acceptable carriers or excipients when used in diagnosis or therapy.

As used herein, the term “isolated cell” refers to a cell that is separated from the molecular and/or cellular components that naturally accompany the cell.

The term “operably linked” as used herein refers to the association of nucleotide sequences on a single nucleic acid molecule, e.g., a vector, or an expression cassette, in a way such that the function of one or more nucleotide sequences is affected by at least one other nucleotide sequence present on said nucleic acid molecule. By operably linking, a nucleic acid sequence is placed into a functional relationship with another nucleic acid sequence on the same nucleic acid molecule. For example, a promoter is operably linked with a coding sequence of a recombinant gene, when it is capable of effecting the expression of that coding sequence. As a further example, a nucleic acid encoding a signal peptide is operably linked to a GOI or a nucleic acid sequence encoding a POI, when it is capable of expressing a protein in the secreted form, such as a preform of a mature protein or the mature protein. Specifically, such nucleic acids operably linked to each other may be immediately linked, i.e., without further elements or nucleic acid sequences in between the nucleic acid encoding the signal peptide and the nucleic acid sequence encoding a POI.

The term “recombinant” as used herein shall mean “being prepared by or the result of genetic engineering. A recombinant host may be engineered to insert one or more nucleotides, polynucleotides or nucleotide sequences, and may specifically comprise an expression vector or cloning vector containing a recombinant nucleic acid sequence, in particular employing nucleotide sequence foreign to the host. A recombinant host cell can be produced by using genetic engineering, i.e., by human intervention, such as to insert a GOI at a certain chromosomal locus. When a host cell is engineered to incorporate a GOI at a safe harbor locus for stable expression of the GOI, the host cell is manipulated such that the host cell has the capability to express such gene at a certain level over a prolonged period of cultivation (e.g., in situ, in vivo, ex vivo or in vitro) which is higher than the expression level of the host cell under the same condition prior to manipulation, or compared to the host cells which are not engineered for GOI expression.

Genetic engineering is conveniently performed by genome editing techniques, homologous recombination or other site-directed recombination technologies such as by Flp-FRT recombination, Cre-Lox recombination, or phage lambda site-specific recombination

Flp-FRT recombination is understood as a site-directed recombination technology and involves the recombination of sequences between short flippase recognition target (FRT) sites by the recombinase flippase (Flp) derived from the 2 µ plasmid of baker’s yeast Saccharomyces cerevisiae.

Cre-Lox recombination is understood as a site-specific recombinase technology. The system consists of a single enzyme, Cre recombinase, which recombines a pair of short target sequences called the Lox sequences.

Phage lambda site-specific recombination employs topoisomerase activity of bacteriophage lambda Int protein introducing single-strand breaks into duplex DNA at recognition sequences.

The term “recombinant” with respect to a cell, GOI or POI as used herein, includes a cell, molecule or product of interest that is prepared, expressed, created or isolated by recombinant means, such as a cell engineered to express a GOI as described herein, a GOI chromosomally integrated into a host cell as described herein, or a POI produced by a host cell transformed to express the POI, as described herein. In accordance with the present invention conventional molecular biology, microbiology, and recombinant DNA techniques within the skill of the art may be employed. Such techniques are explained fully in the literature. See, e.g., Maniatis, Fritsch & Sambrook, “Molecular Cloning: A Laboratory Manual, Cold Spring Harbor, (1982).

The term “sequence identity” of a variant, homologue or orthologue as compared to a parent nucleotide or amino acid sequence indicates the degree of identity of two or more sequences. Two or more amino acid sequences may have the same or conserved amino acid residues at a corresponding position, to a certain degree, up to 100%. Two or more nucleotide sequences may have the same or conserved base pairs at a corresponding position, to a certain degree, up to 100%.

Sequence similarity searching is an effective and reliable strategy for identifying homologs with excess (e.g., at least 50%) sequence identity. Sequence similarity search tools frequently used are e.g., BLAST, FASTA, and HMMER.

Sequence similarity searches can identify such homologous proteins or genes by detecting excess similarity, and statistically significant similarity that reflects common ancestry. Homologues may encompass orthologues, which are herein understood as the same protein in different organisms, e.g., variants of such protein in different different organisms or species.

To determine the % complementarity of two complementary sequences, one of the two sequences needs to be converted to its complementary sequence before the % complementarity can then be calculated as the % identity between the first sequence and the second converted sequences using the above-mentioned algorithm.

“Percent (%) amino acid sequence identity” with respect to an amino acid sequence, homologs and orthologues described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

For purposes described herein, the sequence identity between two amino acid sequences is determined using the NCBI BLAST program version BLASTP 2.8.1 with the following exemplary parameters: Program: blastp, Word size: 6, Expect value: 10, Hitlist size: 100, Gapcosts: 11.1, Matrix: BLOSUM62, Filter string: F, Compositional adjustment: Conditional compositional score matrix adjustment.

“Percent (%) identity” with respect to a nucleotide sequence e.g., of a nucleic acid molecule or a part thereof, in particular a coding DNA sequence, is defined as the percentage of nucleotides in a candidate DNA sequence that is identical with the nucleotides in the DNA sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

Optimal alignment may be determined with the use of any suitable algorithm tor aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomies.org.cn), and Maq (available at maq.sourceforge.net).

The term “stable” as used herein in the context of expression, expressers or expression constructs, means that a host cell is capable of correctly expressing the GOI over a prolonged period of time. A stable expresser is specifically understood to refer to a host cell maintaining the genetic properties, specifically keeping a GOI expression at a high and/or about constant level, even after about 20 generations, preferably at least 30 generations, more preferably at least 40 generations, most preferred of at least 50 generations. Specific embodiments refer to a stable expression during at least 5, or 10, or 20, or 30, or 40, or 50 doublings or passages.

According to a specific aspect, a population of host cells is provided wherein said cells comprise stably integrated into their chromosome a heterologous GOI, wherein on average at least 20%, at least 30%, at least 40%, at least 50%, at least 60% of the cells originating from said population do not lose more than 70%, preferably not more than 50%, of their gene product expression titer over a time period of at least 4 weeks, preferably 8 weeks, preferably 10 weeks, more preferably over a time period of 12 weeks. Expression may be monitored ex-vivo (in cell culture) or in vivo (upon transplantation). As can be shown in the examples, after transfection and identification of stably transfected cells, the amount of cells which do not show a gradual loss in productivity during prolonged culturing is increased when using the cells described herein, i.e., more stable cell clones are obtained from a selected cell population. The stability property can be tested, by cultivating individual cells from said population as cell clones and determining the titer over the indicated time period. The host cell’s expression of the GOI can be determined by various methods, e.g., by ELISA, by Western blotting, by radioimmunoassays, by immunoprecipitation, by assaying for the biological activity of the gene product, or by immunostaining followed by FACS analysis. In a specific aspect, the expression of the GOI is determined by Western blotting.

The stability can be tested by the absence of gene silencing, such as described in Example 3. It can also be tested by culturing cells over extended period of time and monitoring transgene expression levels in vitro or in vivo, by methods known in the art. As transgene insertion may occur in vivo, transgene expression can be monitored in vivo, e.g., by taking patient samples (e.g., blood or biopsies from the relevant tissue). Typically, the expression is considered stable, if transgene expression is about the same (±50% or ±40% or ±30% or ±20% or ±10%) during the observation period.

The stability rates can vary from project to project depending on the expressed gene product. However, the abundance of cells with stable expression characteristics can be significantly increased in the population of successfully transduced host cells. Therefore, the risk that an instable clone which gradually loses productivity during prolonged culturing is chosen for therapy or any other industrial application can be significantly reduced.

Specifically, a stable recombinant host cell line is provided which is considered a great advantage when used for industrial scale production.

Stable expression can be effected upon transduction, e.g., by genome editing or other approaches for targeted transgene insertion known in the art, thereby integrating an expression construct into the host cell’s genome, wherein the targeted insertion is within a predetermined chromosomal region that is considered to be a “safe harbor”, such as within an intergenic region as further described herein. Stable transduction is preferred over a transient one to generate high expressing host cells or clones for expressing a GOI and producing a gene product, or a POI in vivo, or in vitro such as on industrial scale. A population of stable expressers includes a relative high proportion of high and stable producing clones. Stability is particularly increased when incorporating a GOI into a safe harbor locus such as within the intergenic region as described herein, thereby reducing or avoiding transcriptional gene silencing.

In the context of the present invention, the stable expression of a heterologous GOI is achieved by allowing the respective expression construct to be inserted into the intergenic region between two adjacent essential genes, as described herein. Such intergenic region is understood as a hot spot region, meaning that it supports high transcription of introduced genes and that this transcription is stable over time and reproducible for different genes and different culture conditions.

The term “essential gene” is herein understood to include those genes of a host cell coding for an essential polypeptide, and is preferably a gene that has not been shown to be non-essential in the host cell. In one embodiment, the essential gene is a gene whose deficiency renders the host cell non-viable under certain culture conditions because of lower or no expression in the host cell of the essential polypeptide. The essential polypeptide may be a polypeptide able to produce a nutrient which is essential for cell viability (e.g., wherein the essential polypeptide is an enzyme able to produce the nutrient essential for cell viability) or a polypeptide involved in the production of a nutrient which is essential for cell viability (e.g., wherein the essential polypeptide is an enzyme involved in the metabolic pathway which leads to the production of a nutrient essential for cell viability). Yet, the essential gene coding for the essential polypeptide may be a gene whose deficiency renders the host cell non-viable under all conditions and in any type of nutrient medium. The essential polypeptide may be a polypeptide whose lower or no expression in the host cell renders the host cell non-viable under all conditions and in any nutrient medium, such as minimal or complex medium. Preferably, the essential gene coding for an essential polypeptide is a gene essential in eukaryotes coding for a polypeptide essential in eukaryotes. Suitable examples of classes of essential genes include, but are not limited to, genes involved in DNA synthesis and modification, RNA synthesis and modification, protein synthesis and modification, proteasome function, the secretory pathway, cell wall biogenesis and cell division. It is well-known that many genes encoding compounds involved in primary metabolism and metabolic pathways can be essential genes, whose deficiency renders the host cell non-viable.

The term “wildcard host cell” shall mean a host cell, which is prepared by genetic engineering to comprise an artificial or heterologous nucleotide sequence, such as described herein for site-directed insertion of a GOI, and which is ready to incorporate the GOI. A wildcard cell is also understood as an “empty” host cell (i.e., a recombinant host cell without the transgene) that can be used e.g., as cloning cell line for recombinant production technologies. A respective cell can be transfected with a heterologous GOI, e.g., using an appropriate expression vector.

The wildcard cell line is thus a recombinant host cell line, which is characterized for its expression capacity of any desired GOI. This follows an innovative “wildcard” strategy for the generation of producer cell lines, e.g., using site-specific recombinase-mediated cassette exchange or homologous recombination. Such a new host cell facilitates the cloning of a GOI, e.g., into predetermined genomic expression hot spots within days in order to get reproducible, highly efficient production cell lines.

Therefore, the present invention provides for a novel solution to overcome transgene silencing by selecting safe harbour loci in the genome that cannot be silenced. Silencing is a phenomenon that is implemented by establishing repressive marks on DNA or histones (e.g., trimethylation of histone H3 on lysine 9 or 27). Herein described are target regions, each being surrounded by two essential genes. Preferably, the two essential genes are oriented such that their promoters are immediately adjacent to the safe harbour site and face in opposite directions. And preferably, the two promoters are very close to one another, thus ensuring that silencing of the transgene, if it occurred, would silence the neighbouring essential genes. Consequently, silencing of the transgene would lead to cell death, thus implementing a mechanism by which one can select for cell types and cell states in which no silencing of the transgene occurs.

The essential genes are preferably highly essential across multiple cell types and thus belong to the “core essential gene set”, pertaining to genes (i) which are highly essential and thus absolutely required and (ii) which are essential in every cell of a higher organism, such as the human body, in order to make the strategy universally applicable. Suitable pairs of essential genes are ideally close to one another (preferably with their promoter regions), such as to allow the introduction of a transgene in between them. An exemplary selection of around 20 suitable loci, flanked by two highly essential genes is particularly provided.

A transgene can be inserted into the described safe harbour loci in several ways. Those include the use of Zinc finger nucleases in which an array of Zinc finger proteins mediates binding to a specific genomic sequence and Fokl nuclease triggers the DNA double-strand break near the Zinc finger binding site. Alternatively, Transcription Activator-like Effector Nuclease (TALENs) consisting of an array of plant-derived transcription activator-like effectors and Fokl nuclease is equally suited. In addition, DNA- or RNA-guided nucleases can be used. Those include the popular CRISPR enzymes Cas9, Cas12a/ Cpf1 or Cas12b, CasX, but also more complex CRISPR systems such as the Cascade complex. It may also include bacterial Argonautes which have been used for targeted DNA double-strand break induction. The transgene can also be delivered by a combination of a programmable endonuclease with AAV-derived DNA donor, which is often chosen to enhance rates of homologous recombination.

The expression of transgenes from safe harbour loci is a particularly relevant when creating human or other (e.g., CHO) cell lines expressing high levels of gene products, such as antibodies or cytokines, utilized as production cell lines.

It can also be exploited for gene and cell therapy. For instance, it can be used to introduce a chimeric antigen receptor (CAR) into a T cell in a targeted fashion in order to ensure that the CAR is stably expressed and is not silenced during T cell expansion or re-perfusion. Likewise, it can be applied in T cell receptor (TCR) therapy where the endogenous TCR is replaced with an exogenous TCR in order to reprogram the specificity of a T cell and engineer it to recognize a novel target cell (e.g., a tumour cell).

The approach described here can also be utilized to engineer stable induced pluripotent stem (iPS) cell lines which express a transgene to high levels and where transgene expression is unaffected by iPS cell differentiation. The latter remains a significant problem in stem cell biology.

Transgene expression can be monitored at the mRNA or protein level in various ways. mRNA levels can be quantified by quantitative RT-PCR or by RNA sequencing. Protein levels can be determined by Western Blot or ELISA. At the single cell level, protein expression can be determined by flow cytometry. This does not only report the protein level in individual cells, but also reports the fraction of cells in a population that express the transgene. When a transgene is well expressed, its expression can be monitored in single cells by flow cytometry, thus reporting which fraction of a total population expresses the transgene. At the DNA level, the fraction of cells harbouring the transgene can be estimated by digital droplet PCR. Alternatively, one may sort single cells into individual wells of a 96well plate and determine the fraction of transgene-positive cells by a PCR-based approach.

The issue of gene silencing can be addressed in two ways:

Human iPS cells can be utilized and an expression cassette introduced into the candidate safe harbour locus using CRISPR/Cas9. The expression cassette contains a strong promoter (e.g., EF1A) driving eGFP or mCherry, thus ensuring that the transgene is strongly expressed. Once a stable clone has been isolated in which transgene integration has been verified by PCR, iPS cells are differentiated into various lineages (e.g., neurons or cardiomyocytes). Transgene expression is monitored by FACS throughout the differentiation process to ensure that expression is stably maintained and not subject to silencing.

A human cell line (e.g., HEK293, Jurkat, U937, Hela or human iPS cells) is used to introduce an expression cassette into the candidate safe harbour locus using CRISPR/Cas9. The expression cassette contains eGFP driven by a promoter (e.g., SFFV, CMV or EF1As) that is flanked by Tetracycline resistance (tetO) sequences. In the absence of TetR-KRAB, eGFP is strongly expressed in the recipient cells and a stable population or a clonal cell line can be obtained by FACS sorting. Introduction of TetR-KRAB leads to the silencing of eGFP. However, the silencing will spread to the neighbouring promoters governing two essential genes which leads to the death of the cells in which effective silencing occurs. Consequently, one would expect that all cells that lose eGFP expression will die. Survival of a significant fraction of eGFP-negative cells indicates that, contrary to such expectation, silencing of this locus can indeed occur.

The foregoing description will be more fully understood with reference to the following examples. Such examples are, however, merely representative of methods of practicing one or more embodiments of the present invention and should not be read as limiting the scope of invention.

### EXAMPLES

### Example 1: CRISPR Screen to Fine Map the Dual Essential Gene Loci

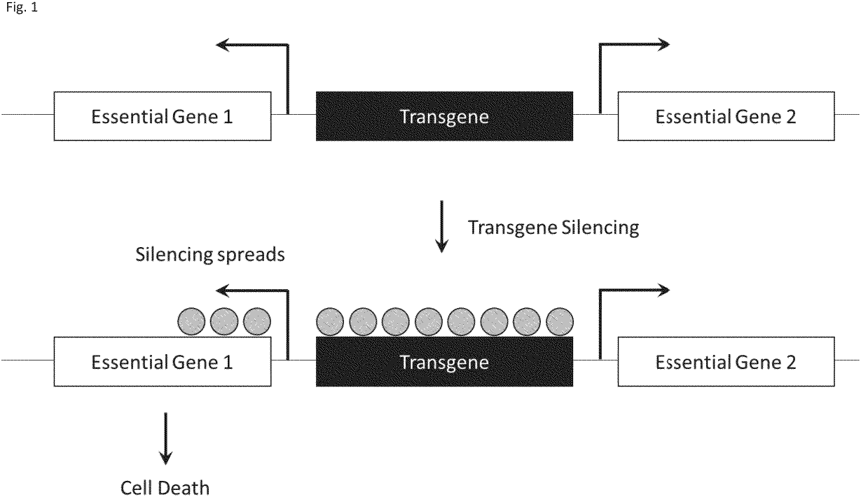

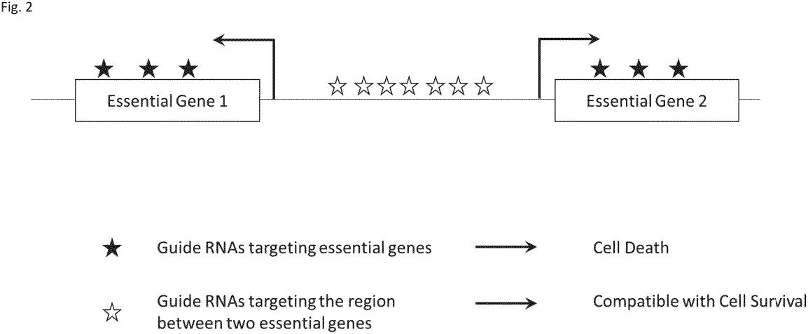

Transgene insertion between two essential genes could be harmful to the recipient cell as it may alter the expression of at least one of the two essential genes. Hence, it is assessed which guide RNAs can be utilized for transgene insertion without compromising cell fitness. To identify these, the entire region between the transcriptional start sites of each essential gene pair is tiled with all possible guide RNAs (FIG. 2) and their fitness phenotype is assessed in a dropout screen. As references, guide RNAs targeting the coding regions of the essential genes in question are included, thus confirming that these genes are indeed essential. Negative controls are also included, i.e., guide RNAs targeting non-essential genes or intergenic regions.

### Example 2: Targeting Safe Harbour Loci and Addressing the “Silenceability” Experimentally

Two human gene pairs have been identified and targeting the respective intergenic regions is evaluated. The two gene pairs are: MED20/ BYSL and FTSJ3/ PSMC5.

The first pair of essential genes: the first gene is FTSJ3 (GeneID: 117246) and the second gene is PSMC5 (GeneID: 5705), intergenic region: SEQ ID NO:8.

The second pair of essential genes; the first gene is MED20 (GeneID: 9477) and the second gene is BYSL (GeneID: 705), intergenic region: SEQ ID NO:9.

For each of them, the region between the two essential genes is targeted using the following guide RNAs:

MED20 guide RNA 1 (SEQ ID NO:22):
GGGCGUGUCUCGGCACCCCUGUUUCAGAGCUAUGCUGGAAACAGCAUAGCAAGUUGAAAUAAGGCUAGUCCGUUAUCAACUUGAAAAAGUGGCACCGAGUCGGUGCUUUUU

MED20 guide RNA 2 (SEQ ID NO:23):
GAGCUCCCGGGUUCCGGAGCGUUUCAGAGCUAUGCUGGAAACAGCAUAGCAAGUUGAAAUAAGGCUAGUCCGUUAUCAACUUGAAAAAGUGGCACCGAGUCGGUGCUUUUU

FTSJ3 guide RNA 1 (SEQ ID NO:24):
GGGGCGGCUACUCGAGUUCAGUUUCAGAGCUAUGCUGGAAACAGCAUAGCAAGUUGAAAUAAGGCUAGUCCGUUAUCAACUUGAAAAAGUGGCACCGAGUCGGUGCUUUUU

FTSJ3 guide RNA 2 (SEQ ID NO:25):
GAAUUCCGGGUCAAUGGGCGGUUUCAGAGCUAUGCUGGAAACAGCAUAGCAAGUUGAAAUAAGGCUAGUCCGUUAUCAACUUGAAAAAGUGGCACCGAGUCGGUGCUUUUU

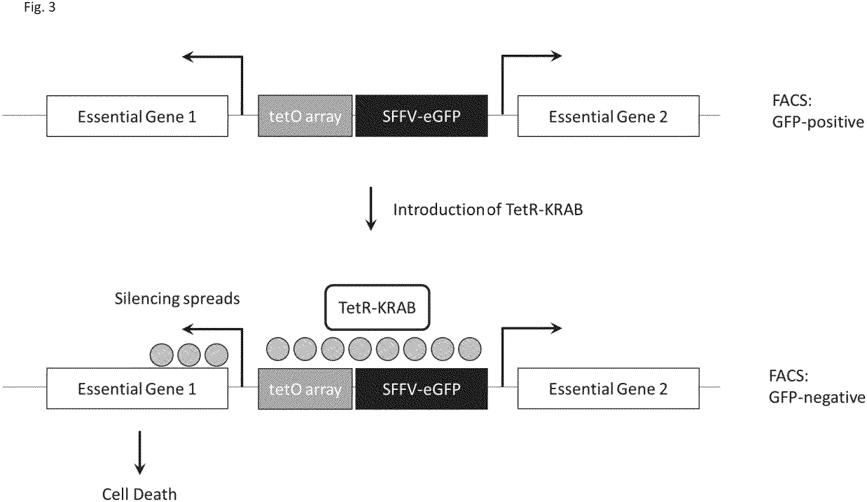

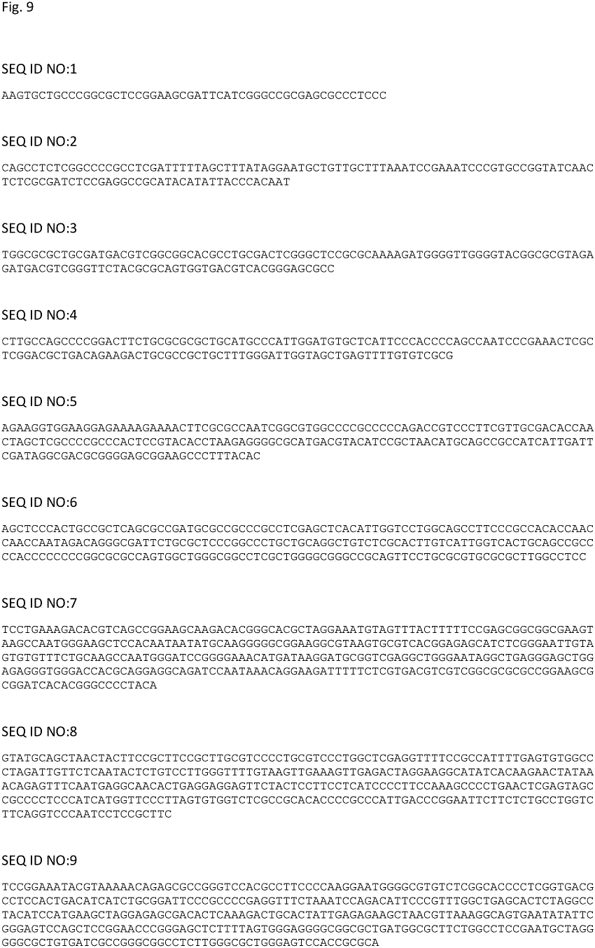

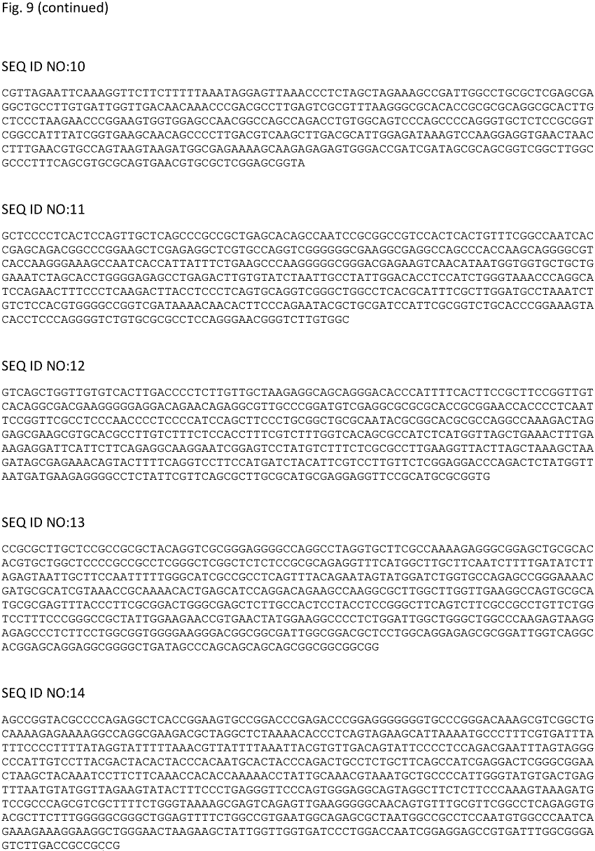

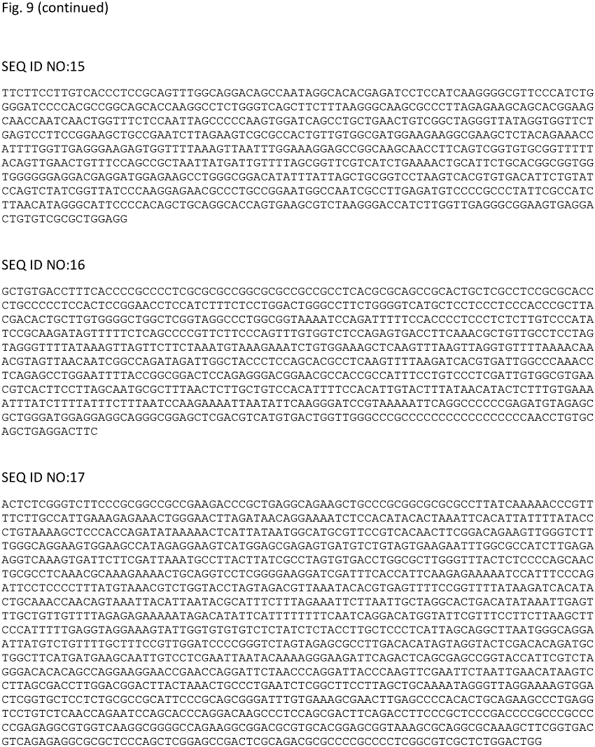

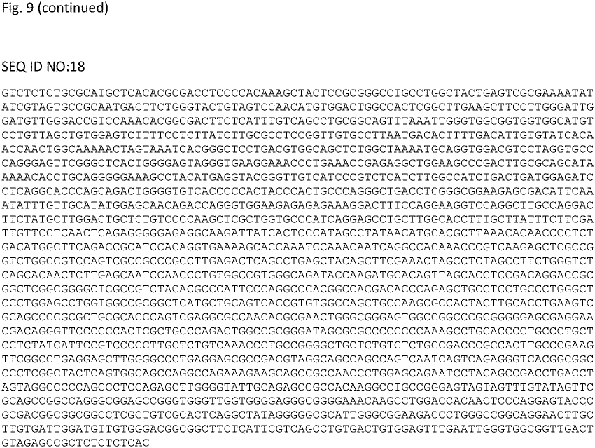

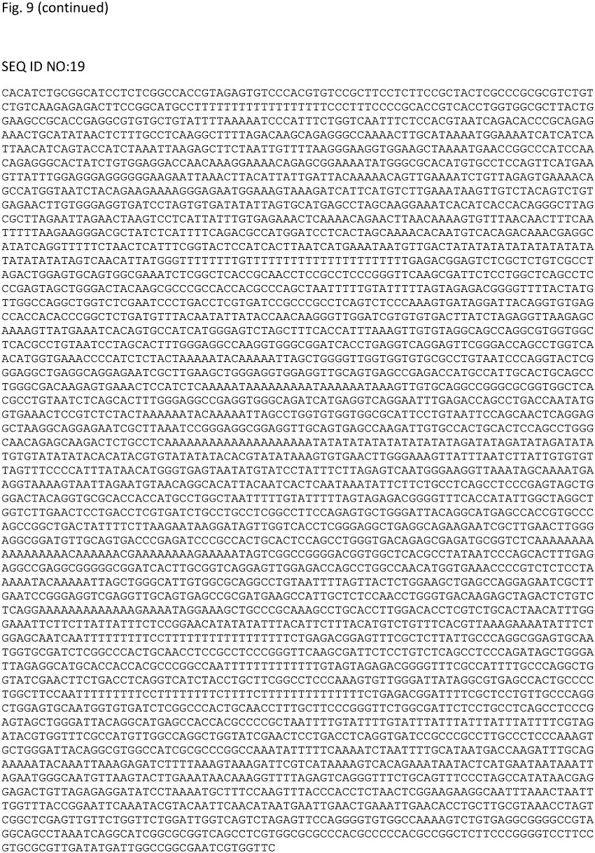

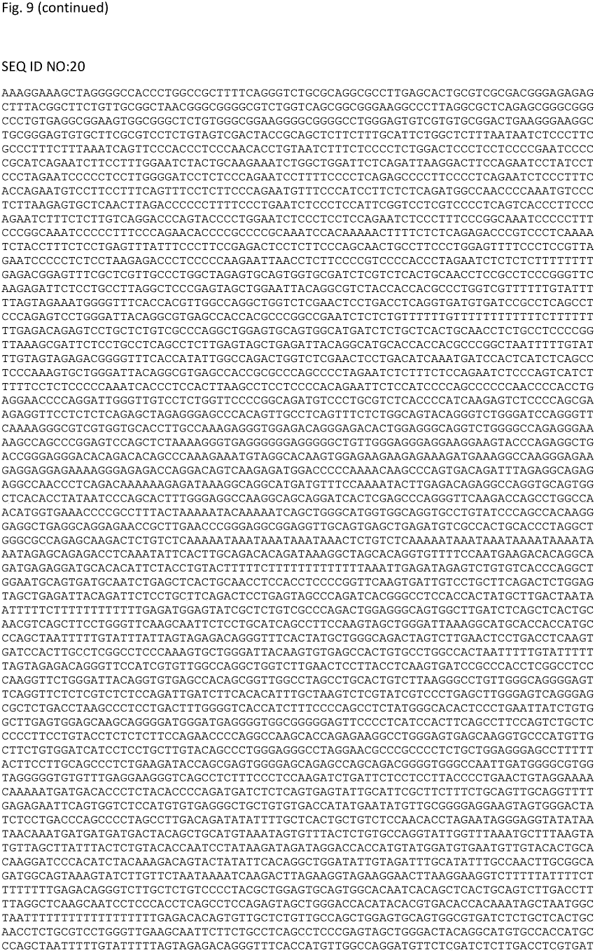

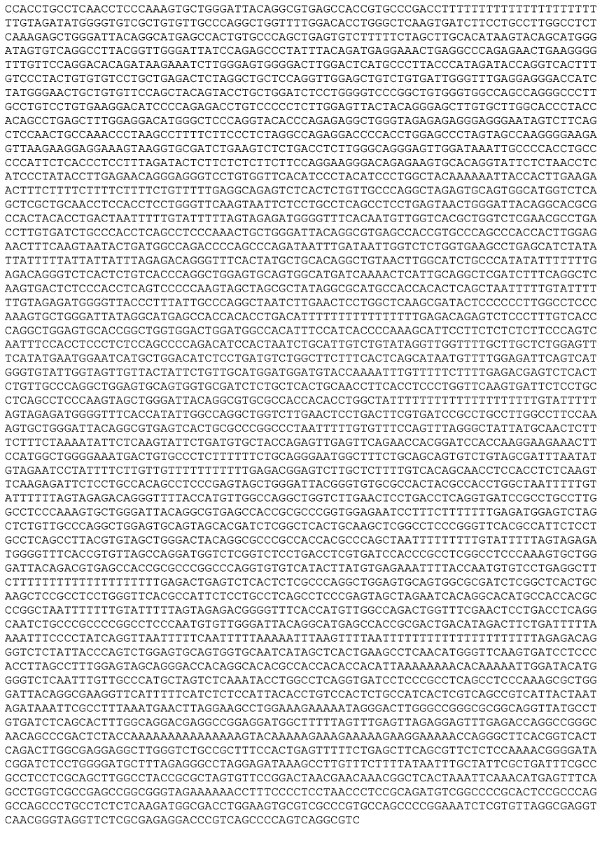

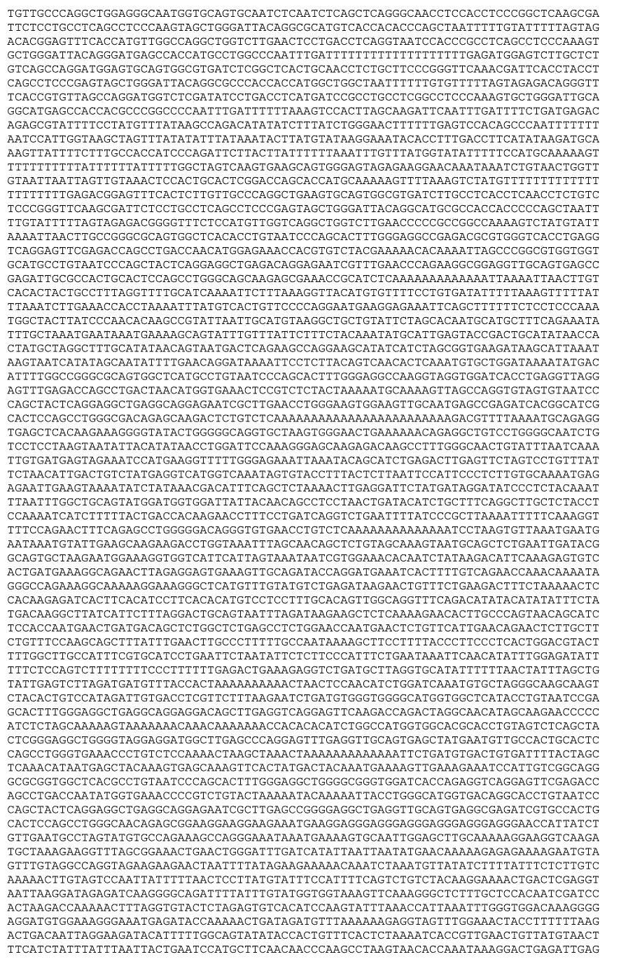

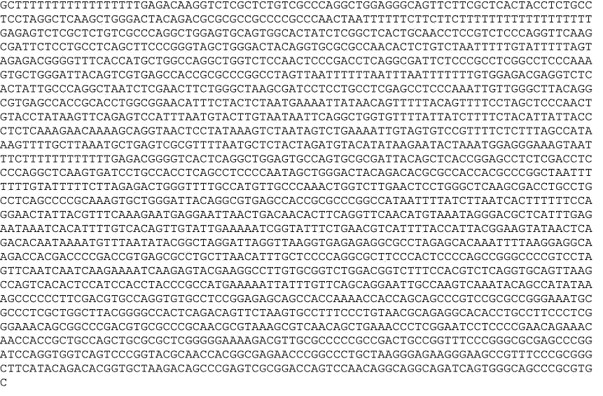

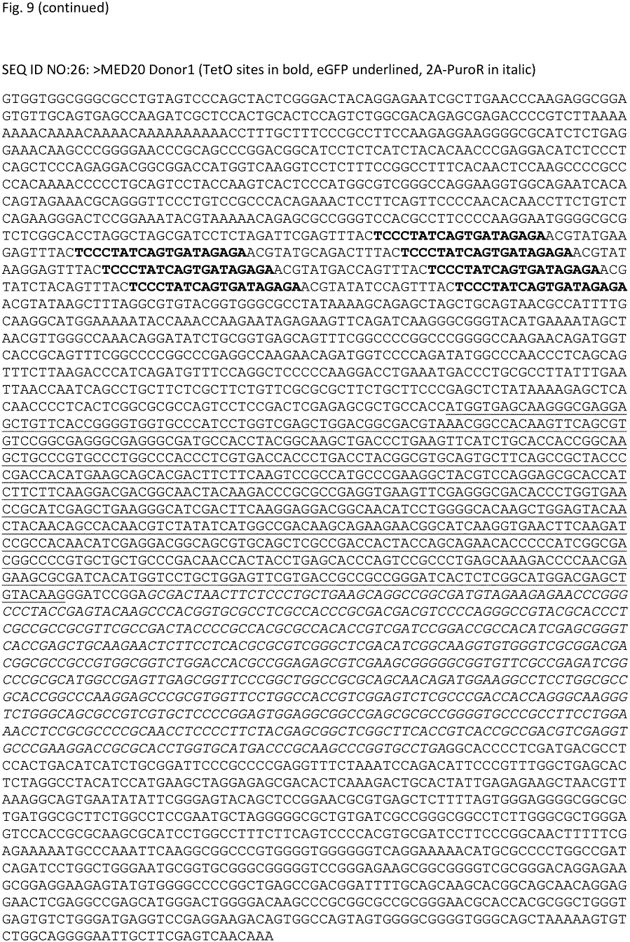

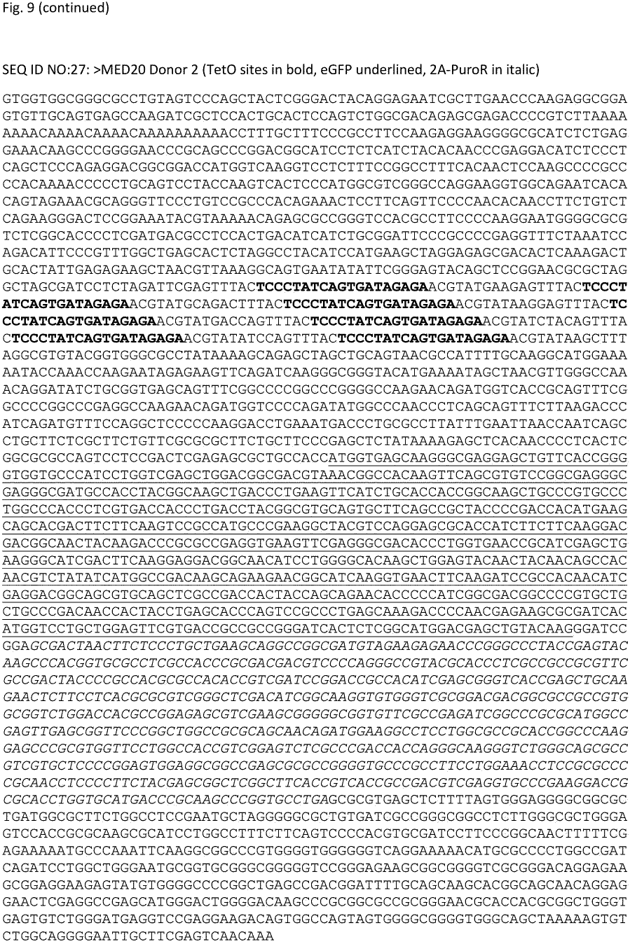

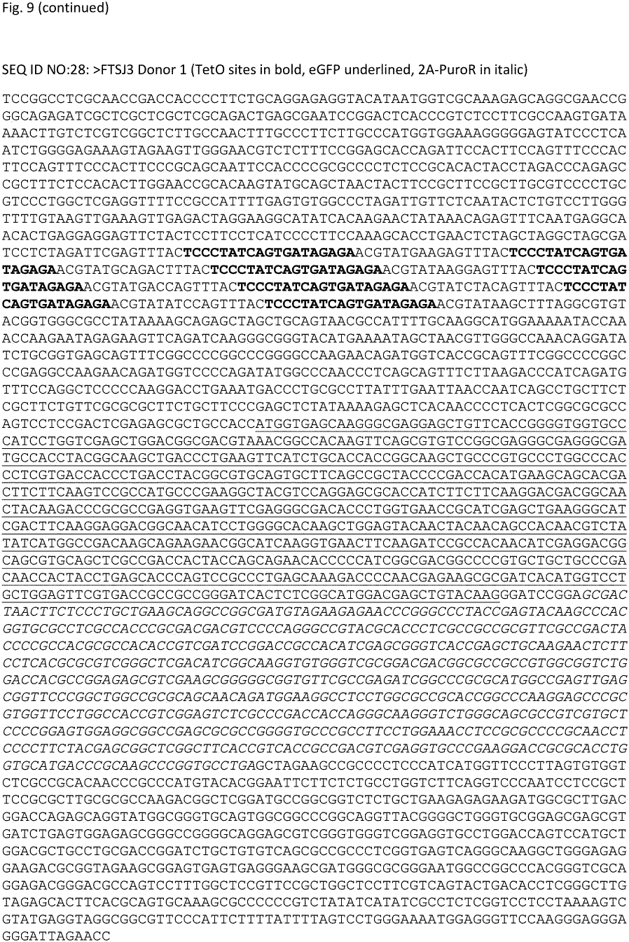

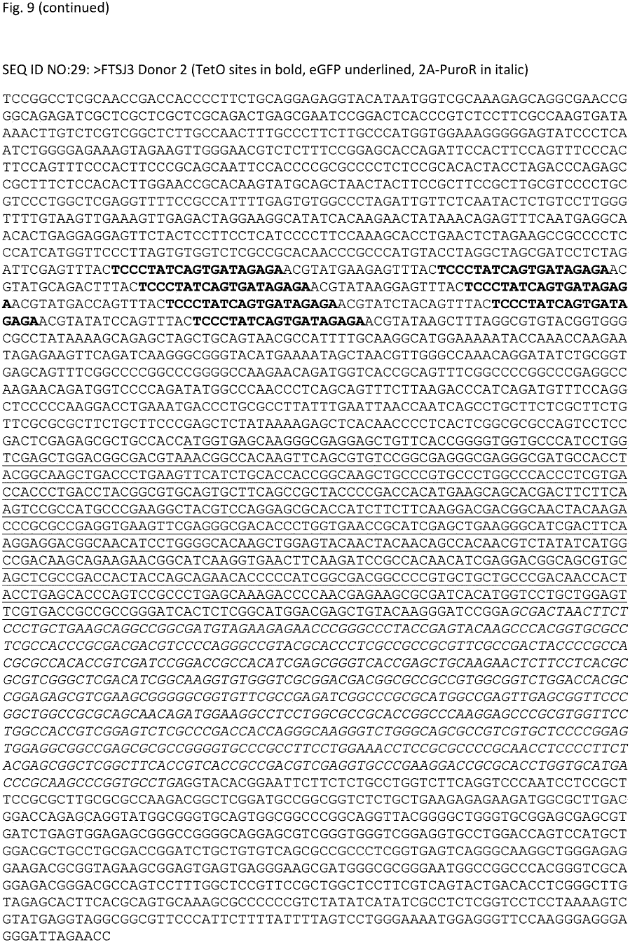

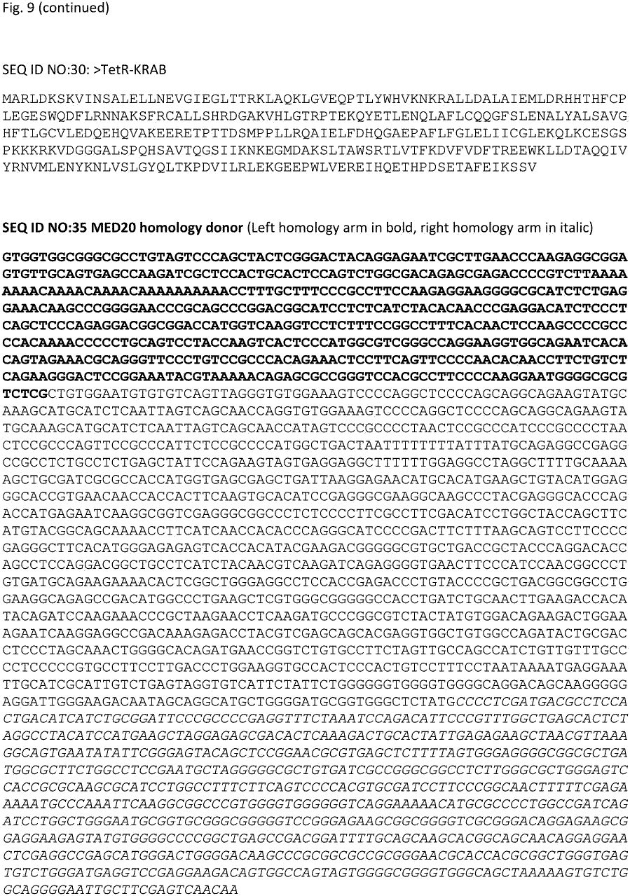

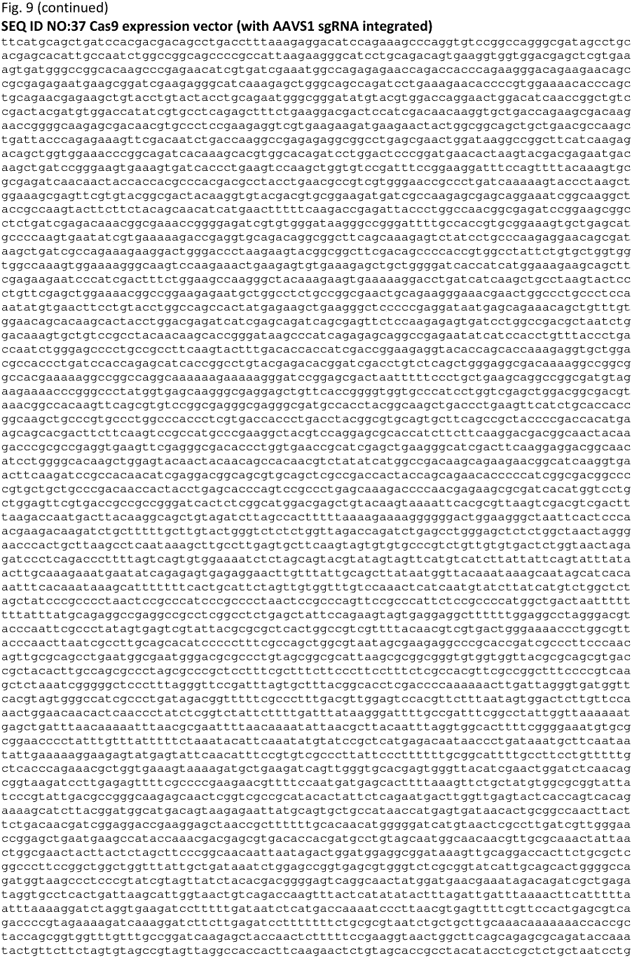

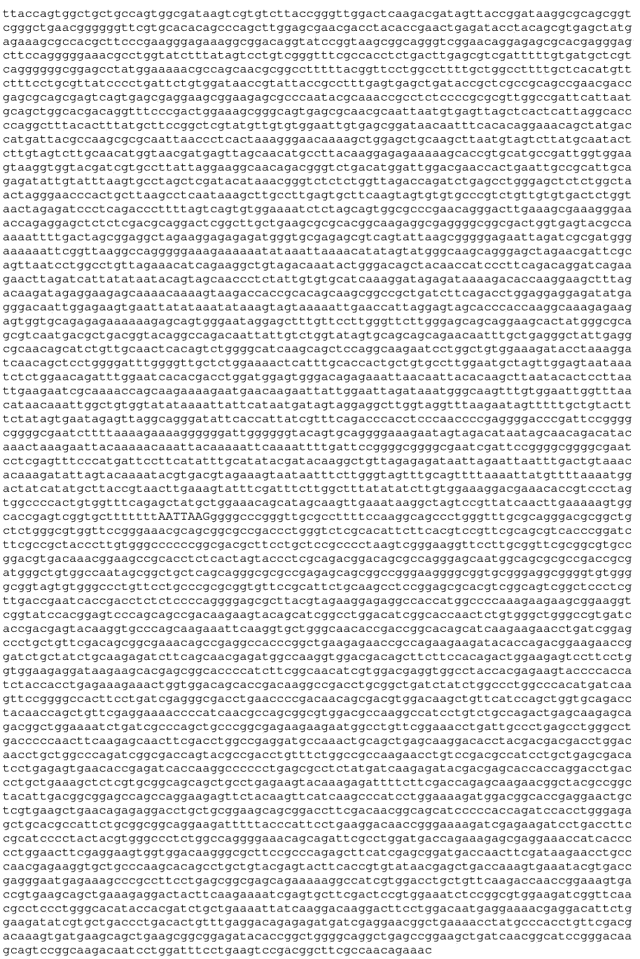

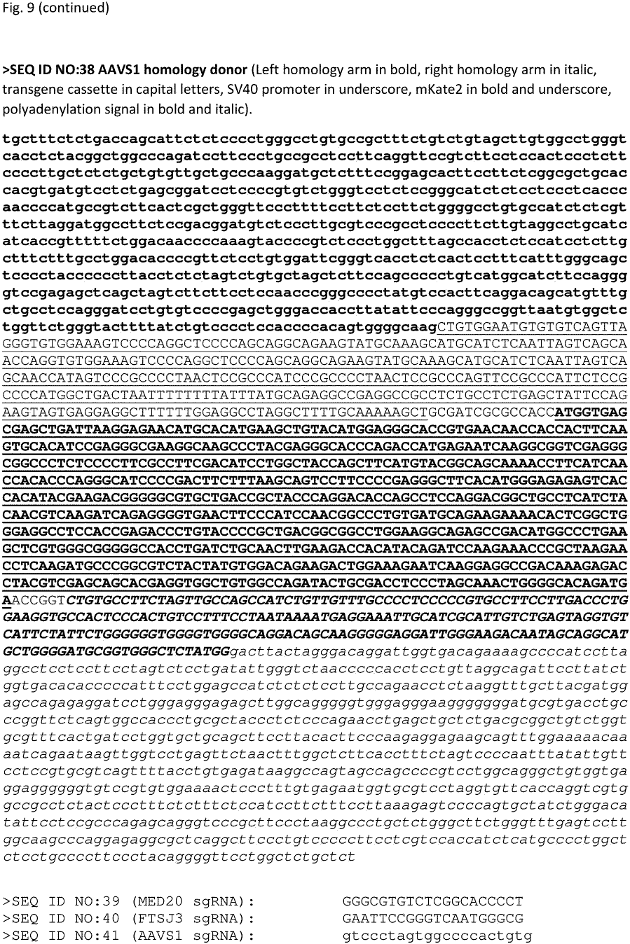

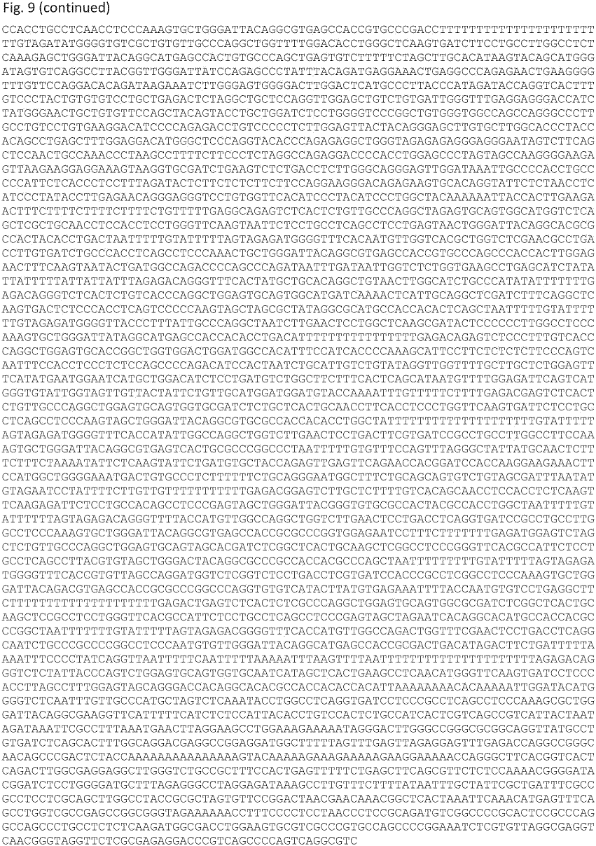

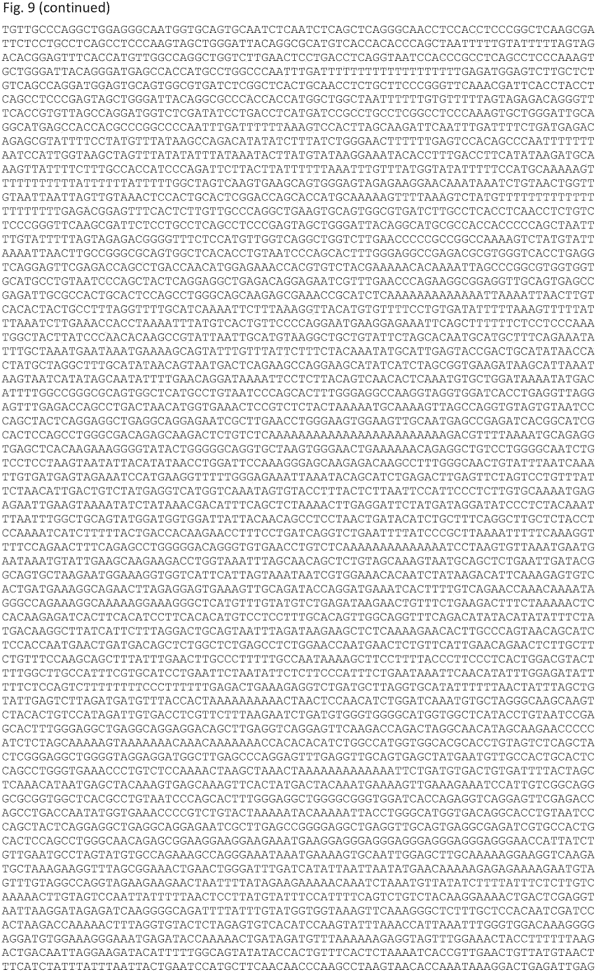

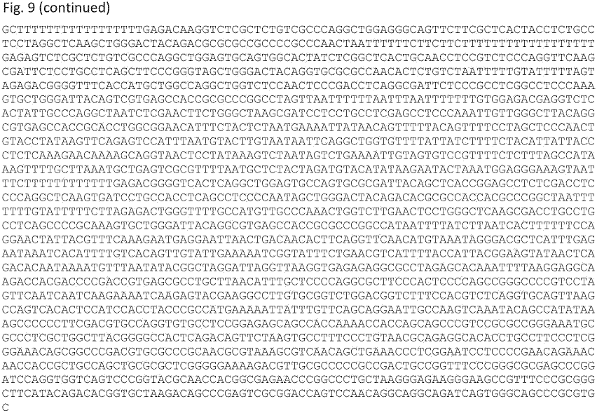

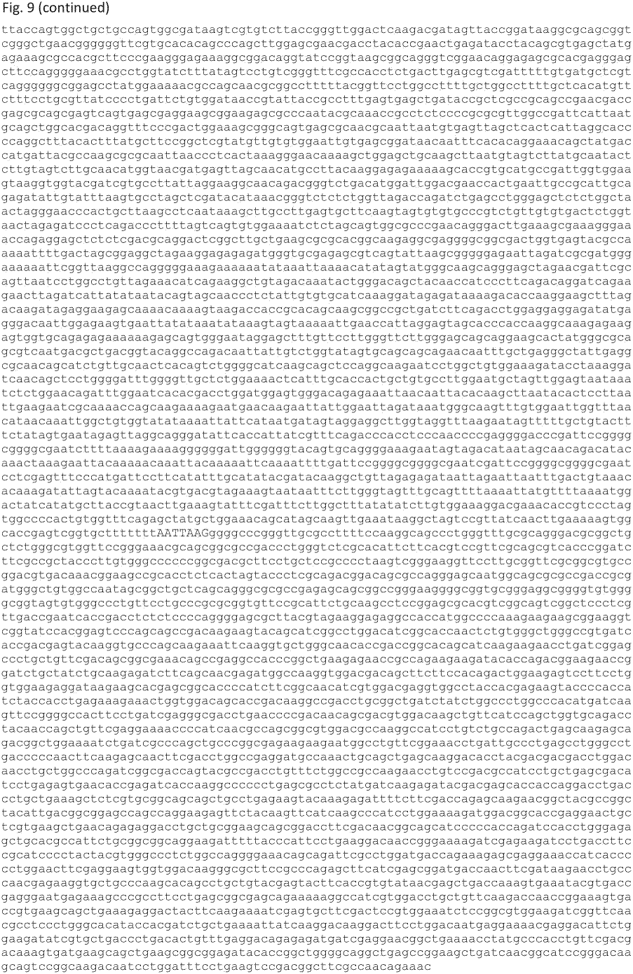

An expression cassette (FIG. 3) is introduced into each of the specified loci by homology-directed repair. The expression cassette harbours eGFP linked to Puromycin resistance via a 2A peptide, the expression of which is driven by an SFFV promoter. The SFFV promoter is flanked by an array of seven tetO sites as specified in the respective donor sequences, FIG. 9:

SEQ ID NO:26, >MED20 Donor1 (TetO sites in bold, eGFP underlined, 2A-PuroR in italic).

SEQ ID NO:27: >MED20 Donor 2 (TetO sites in bold, eGFP underlined, 2A-PuroR in italic).

SEQ ID NO:28: >FTSJ3 Donor 1 (TetO sites in bold, eGFP underlined, 2A-PuroR in italic).

SEQ ID NO:29: >FTSJ3 Donor 2 (TetO sites in bold, eGFP underlined, 2A-PuroR in italic).

HEK293 cells are transduced with a Cas9 expression plasmid, alongside with the following combination of guide RNA expression plasmid and donor plasmid:
MED20 guide RNA 1 with MED20 donor 1
MED20 guide RNA 2 with MED20 donor 2
FTSJ3 guide RNA 1 with FTSJ3 donor 1
FTSJ3 guide RNA 2 with FTSJ3 donor 2

Following expression of Cas9 and the guide RNA, the donor is incorporated by homology-directed repair at the target site. Cells bearing the cognate insertion of the transgene cassette can be selected for using puromycin and enriched by subsequent FACS sorting. Ultimately, clones bearing the transgene insertion are obtained by limiting dilution and will be genotyped using PCR.

Once a stable cell line (monoclonal or polyclonal) has been obtained, cells are transfected with an expression cassette harbouring the TetR-KRAB construct specified in FIG. 8, SEQ ID NO:30.

The TetR-KRAB construct, upon introduction in the recipient cell, is expressed and binds to the array of tetO sites. Binding of TetR-KRAB induces silencing of the SFFV promoter, thus effectively silencing the eGFP transgene. However, silencing is spread to the adjacent promoters governing the two essential genes (MED20/ BYSL or FTSJ3/ PSMC5). Consequently, cells in which eGFP silencing has occurred die.

Surviving cells are assayed by flow cytometry for GFP positivity. To prove that the novel safe harbour loci that are located between two essential genes are functional, there are no surviving cells that are GFP-negative and all surviving cells maintain GFP expression.

### Example 3 Assessing Transgene Silencing in Human iPS Cells

Human iPS cell lines such as BOB-C or KOLF2 are used, which are obtained from the HipSci consortium. A GFP reporter under the control of a constitutive promoter (e.g., CMV, SFFV, EF1As, EF1A) is introduced into the safe harbour locus of interest. Two exemplary combinations of guide RNA and donor vector are provided in Example 2. In contrast to Example 2, the donor vector does not harbour any TetO sites.

Following introduction via CRISPR-Cas9 as specified in Example 2, a stable population of cells (polyclonal or monoclonal) harbouring the transgene at the desired locus is obtained. This population is differentiated into various lineages using protocols known in the art (Volpato V et al. Stem Cell Reports. 2018;11(4):897-911; Mummery CL et al. Circ Res. 2012;111(3):344-358). Transgene expression is assessed prior to differentiation and at the various stages of differentiation (early, late, terminal). Depending on the target cell type, differentiation takes from 15 days to 120 days, and differentiation is monitored using suitable markers known in the art. To prove that no silencing occurs, transgene expression is stably detected (e.g., by flow cytometry) during the entire course of the experiment. Clones in which silencing of the transgene occurred succumb to cell death as the silencing inevitably spreads to at least one of the neighbouring essential genes (FIG. 1).

### Example 4: CRISPR Screen

Essential genes are those genes whose disruption leads to a loss of fitness or viability. In this example, it was assessed which of the 21 gene pairs nominated in Table 1 is essential. In addition, the region between the two respective essential genes was targeted. If targeting with CRISPR/ Cas9 in these intergenic regions is feasible, one would expect guide RNAs to behave as neutral, i.e., cells harbouring these guide RNAs will not have a fitness phenotype.

To test this at scale for the ten gene pairs mentioned above, a suitable customized sgRNA library was designed. In this library, each essential gene was targeted with approximately 10 independent sgRNAs. sgRNAs were selected to target coding exons of the respective genes. In addition, each intergenic region was targeted with as many sgRNAs as possible, i.e., each sgRNA starting with a G and harbouring an NGG PAM adjacent to its sequence was selected. In total, our library had the following composition:

TABLE 2

| ID | Gene 1 | Gene 2 | # of sgRNAs targeting gene 1 | # of sgRNAs targeting gene 2 | # of sgRNAs targeting intergenic region |
|---|---|---|---|---|---|
| NO 1 | NFS1 | ROMO1 | 10 | 10 | 4 |
| NO 2 | MED22 | RPL7A | 10 | 10 | 11 |
| NO 3 | DDX51 | NOC4L | 10 | 10 | 9 |
| NO 4 | CENPK | PPWD1 | 9 | 10 | 13 |
| NO 5 | ORC1 | PRPF38A | 10 | 10 | 24 |
| NO 6 | POLR3K | SNRNP25 | 10 | 10 | 28 |
| NO 7 | COPE | DDX49 | 10 | 10 | 31 |
| NO 8 | FTSJ3 | PSMC5 | 10 | 10 | 30 |
| NO 9 | MED20 | BYSL | 10 | 10 | 49 |
| NO 10 | AURKA | CSTF1 | 10 | 10 | 42 |
| NO 11 | NUP88 | RPAIN | 10 | 10 | 71 |
| NO 12 | ATP6V1D | EIF2S1 | 10 | 10 | 47 |
| NO 13 | POLR2I | TBCB | 10 | 10 | 63 |
| NO 14 | UFD1 | CDC45 | 10 | 10 | 29 |
| NO 15 | CCDC115 | IMP4 | 10 | 10 | 71 |
| NO 16 | NAA50 | ATP6V1A | 9 | 10 | 71 |
| NO 17 | SART3 | ISCU | 10 | 10 | 94 |
| NO 18 | C1orf109 | CDCA8 | 10 | 10 | 215 |
| NO 19 | POLR3A | RPS24 | 10 | 4 | 114 |
| NO 20 | RPS16 | SUPT5H | 9 | 10 | 402 |
| NO 21 | RPS29 | LRR1 | 3 | 10 | 67 |

The sgRNA library also targeted a set of 10 non-essential genes with 10 sgRNAs each.

The sgRNA was cloned as is known in the field. In brief, a pooled oligonucleotide library comprising 1989 sgRNAs was cloned into the recipient sgRNA expression vector. Lentivirus was manufactured using Lenti-X cells (based on 293T cells) and RKO cells bearing Cas9 were infected at low multiplicity of infection. After 2 days, cells were quality controlled by flow cytometry to retrieve a population of cells that was 10.2 % GFP-positive. Cells harboring sgRNAs were enriched by puromycin selection (at 0.5 µg/ml concentration).

Cells were harvested at day 3 or day 21 and genomic DNA was isolated. The sgRNA cassette was amplified by two rounds of PCR using the following primer pairs: Round 1:

PCR1_1_FW, SEQ ID NO:31:
ACACGACGCTCTTCCGATCTACATAACGGTGTGGAAAGGACGAAACACCG

(for amplification of gDNA from day 3) Or

PCR1_2_FW, SEQ ID NO:32:
ACACGACGCTCTTCCGATCTTAGTTACGGTGTGGAAAGGACGAAACACCG

(for amplification of gDNA from day 21)

PCR1_REV (NEW SEQ ID NO:49):
GGTCTAACCAGAGAGAGCCAG

Round 2:

PCR2_FW, SEQ ID NO:33:
AATGATACGGCGACCACCGAGATCTACACTCTTTCCCTACACGACGCTCTTCCGA
TCT

PCR2_REV, SEQ ID NO:34:
CAAGCAGAAGACGGCATACGAGATGGTCTAACCAGAGAGAGCCAG

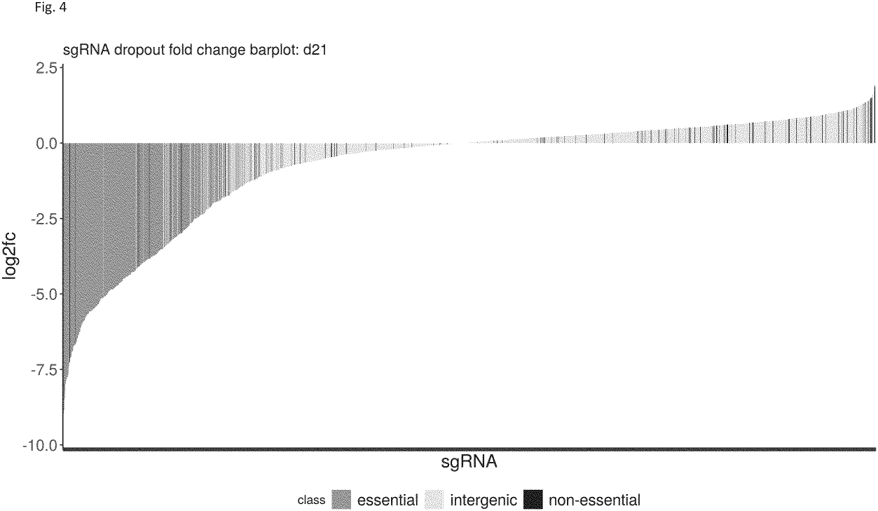

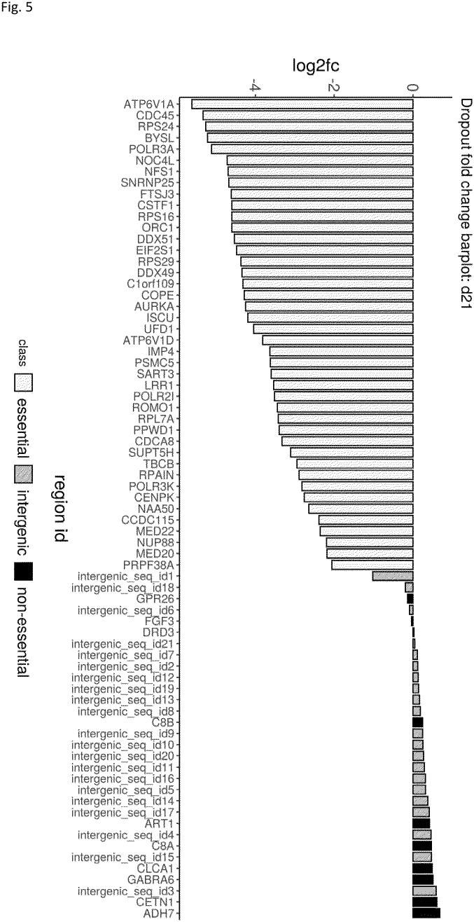

PCR products were subjected to next generation sequencing and the abundance of each sgRNA was quantified by calculating the log fold change (day 21/day 3; FIG. 4). The figure shows a strong depletion of cells bearing sgRNAs targeting essential genes (highlighted). This suggests that Cas9 was functional in the cells tested here and that genes marked as essential are effectively depleted using CRISPR/Cas9.

Next, an aggregated score/ log fold change was calculated on a per gene/ locus basis. The aggregated log fold change for each gene/locus was calculated based on individual sgRNA counts using the MAGeCK software (version 0.5.9.2).

Plotting the fold changes for all genes/ loci (FIG. 5), it was noted that essential genes were clearly depleted from the library, whereas non-essential genes were distributed randomly amongst the intergenic loci. This suggests that most intergenic loci selected here tolerate the cleavage by Cas9 and the subsequent repair by the endogenous NHEJ machinery. It also implies that many of the loci chosen here tolerate at least small insertions or deletions, such as the ones that arise from NHEJ. Importantly, one intergenic region did not tolerate CRISPR/Cas9 cleavage and of note, this region, SEQ ID NO:1, pertains to the gene pair where transcriptional start sites of the two essential genes are very close to one another (52 bp), leaving very little room for insertions/ deletions. Conversely, it suggests that all other loci (SEQ ID NO:2 to SEQ ID NO:21) may tolerate CRISPR/Cas9 cleavage. This is surprising, particularly since this experiment establishes that all of the genes chosen here are clearly essential in RKO cells.

### Example 5: Transgene Insertion at Two Selected Loci

To assess the feasibility of inserting a transgene between two essential genes, two of the selected loci were used: The first one comprised the intergenic region between the genes FTSJ3 and PSMC5 (SEQ ID NO:8), the second one comprised the intergenic region between the genes MED20 and BYSL (SEQ ID NO:9).

It was decided to utilize Lenti-X 293T cells (Takara Bio) because they are easy to transfect. In brief, 1.2 million Lenti-X 293T cells were seeded per well of a 6-well plate in the morning. In the afternoon, cells were co-transfected with 500 ng of plasmid containing a fluorescent marker (SV40-mKate2-pA; obtained by gene synthesis flanked by the respective homology arms for integration by HDR into the different safe harbor loci (SEQ ID NO:35 and SEQ ID NO:36) as well as 1500 ng of a plasmid encoding SpCas9 and an sgRNA targeting the specific genomic region (SEQ ID NO:37). We included a donor targeting the AAVS1 locus (SEQ ID NO:38) which is a commonly used safe harbor locus. sgRNAs used for CRISPR/ Cas9-mediated targeting are depicted as SEQ ID NO:39, SEQ ID NO:40 and SEQ ID NO:41 (sequences provided in FIG. 9).

After 10 days of culture and expansion, mKate2-positive cells were sorted on a BD FACSAria III system and plated in T25 flasks. Cells were cultured and expanded for 3 weeks followed by DNA extraction of 5 million cells for each condition with the Monarch Genomic DNA Purification Kit (#T3010; NEB) according to manufacturer’s instructions. Genotyping PCRs were performed using Phusion High-Fidelity DNA Polymerase (#M0530; NEB) with the indicated primer pairs.

PCRs conducted

| PCR Name | Primer Forward | Primer Reverse | Amplicon size (bp) |
|---|---|---|---|
| AAVS1-mKate2 | AAVS1-genomic | mKate2-insert1 | 1539 |
| MED20-mKate2 | MED20-genomic1 | mKate2-insert1 | 1422 |
| mKate2-MED20 | mKate2-insert2 | MED20-genomic2 | 1481 |
| FTSJ3-mKate2 | FTSJ3-genomic1 | mKate2-insert1 | 1310 |
| mKate2-FTSJ3 | mKate2-insert2 | FTSJ3-genomic2 | 1276 |

### Primers Used for Genotyping

>AAVS1-genomic, SEQ ID NO:42: GTCTGGTCTATCTGCCTGGC

>MED20-genomic1, SEQ ID NO:43: AAACAGACACAAGCGGGTCT

>MED20-genomic2, SEQ ID NO:44: TCCTGCTGCAATCGGAGAAG

>FTSJ3-genomic1, SEQ ID NO:45: GTTACGAACCATCCCCCTGG

>FTSJ3-genomic2, SEQ ID NO:46: ACCCTTCCTAGCTCCCTCTG

> mKate2-insert1, SEQ ID NO:47: GGTAGCCAGGATGTCGAAGG

> mKate2-insert2, SEQ ID NO:48: CCGGCGTCTACTATGTGGAC

PCR reaction

| | |
|---|---|
| Component | Amount |
| Nuclease-free water | 20 µl |
| 5X Phusion HF Buffer | 4 µl |
| 10 mM dNTPs | 0.4 µl |
| 10 µM Forward Primer | 1 µl |
| 10 µM Reverse Primer | 1 µl |
| Genomic DNA | 50 ng |
| DMSO | 0.6 µl |
| Phusion DNA Polymerase | 0.2 µl |

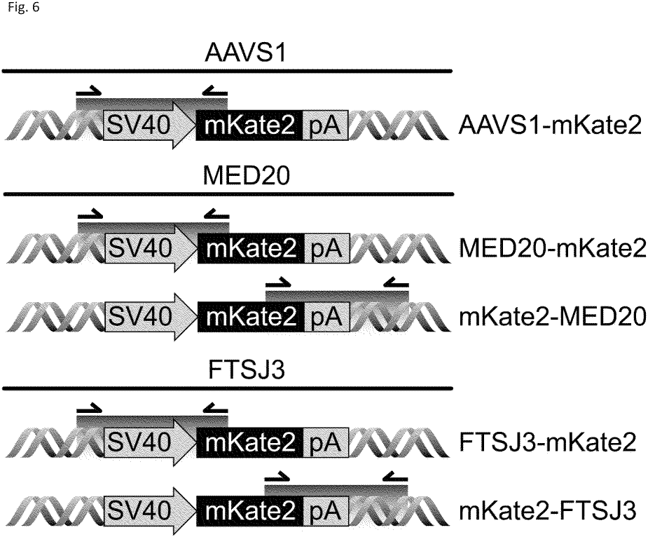

A schematic of the engineered loci, alongside with the location of the primers used for genotyping, is shown in FIG. 6. For each of the loci of interest (MED20/ BYSL or FTSJ3/ PSMC5), it was chosen to confirm on-target integration using two different primer pairs. One of them targets the 5’ end of the inserted transgene, the other one targets the 3’ end of the inserted transgene.

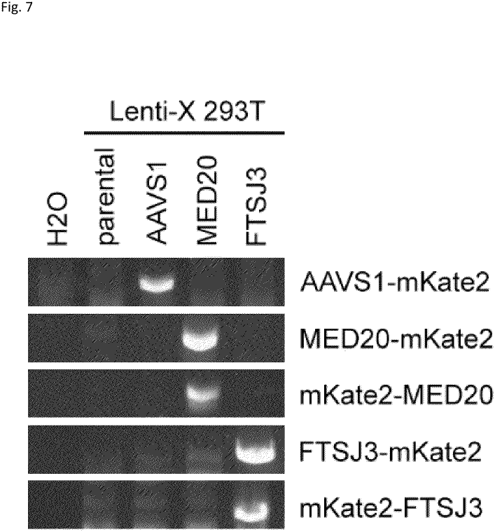

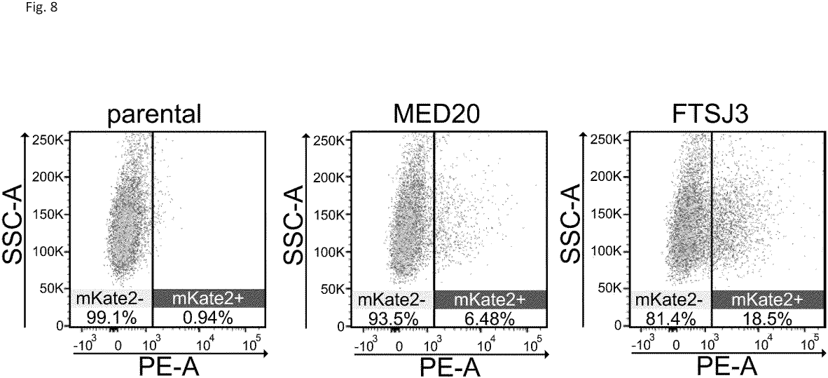

FIG. 7 shows the PCR products obtained in the genotyping reactions described above. All PCRs were conducted across all cell lines, thus establishing the specificity of the PCRs conducted here. PCRs confirm the insertion of the SV40-mKate2-pA transgene at all loci tested here including AAVS1 (as a reference point), MED20/ BYSL and FTSJ3/ PSMC5. They thus establish the technical feasibility of targeting the loci described here. Importantly, the transgene cassette also contained active gene regulatory elements such as a promoter and polyadenylation signal, the insertion of which did not interfere with the expression of the neighbouring essential genes. This suggests that similar cassettes can be inserted at the loci specified here without affecting cellular fitness.

Finally, the expression of the transgene mKate2 was also assessed in the cells in which the transgene had been inserted by CRISPR/Cas9 (see above). To do so, 10,000 cells were subjected to flow cytometry analysis on a BD LSR Fortessa™ Flow Cytometer with default settings for Phycoerythrin (PE) measurement to assess mKate2 expression (FIG. 8). In the cells in which the transgene had been introduced into the MED20/ BYSL locus, mKate2 expression was detected in 6.5% of the cells. In cells bearing a transgene in the intergenic region between FTSJ3 and PSMC5, mKate2 was even detected in 18.5% of the cells. Importantly, these data were recorded at day 28 post transduction, excluding the possibility that transgene expression arose from an episomal plasmid.

Overall, this example indicates that transgene insertion at the two loci picked here is feasible and that the transgene can be expressed stably over time and to levels detectable by flow cytometry.

1. An isolated mammalian host cell comprising a heterologous gene of interest (GOI) chromosomally integrated at a target site within an intergenic region between a pair of adjacent essential genes, wherein each of the adjacent essential genes of said pair has a transcription start site (TSS), and wherein the distance between the TSSs is less than 20,000 nucleotides (nt).

2. The host cell of claim 1, wherein the distance between the TSSs is at least 80 nt.

3. The host cell of claim 1, wherein the length of the intergenic region is less than 20,000 nt.

4. The host cell of claim 1, wherein the intergenic region comprises a nucleic acid sequence which is at least 90% identical to any one of SEQ ID NOs:1-21.

5. The host cell of claim 1, wherein the essential genes of the pair are oriented such that their promoters face in opposite directions.

6. The host cell of claim 1, wherein the GOI is comprised in a heterologous expression cassette integrated at the target site.

7. The host cell of claim 1, wherein the host cell is
a hematopoietic stem cell, an embryonic stem cell, a pluripotent stem cell, an induced pluripotent stem cell, an endothelial cell,
or an immune effector cell selected from a Natural Killer (NK) cell, a microglial cell, a macrophage, and a T cell, such as a cytotoxic T lymphocyte (CTL), a regulatory T cell, or a T helper cell.

8. The host cell of claim 7, wherein the host cell is an immune effector cell comprising a chimeric antigen receptor (CAR), and wherein the GOI encodes the CAR expressed by said immune effector cell.

9. A preparation comprising a population of mammalian host cells, wherein at least 1% of the cells are the host cell of claim 1 .

10. An in vitro host cell culture comprising a host cell of claim 1, maintained under conditions sufficient for expression of the GOI.

11. A method of producing a gene product encoded by a gene of interest (GOI), the method comprising transforming a host cell to chromosomally integrate the GOI within an intergenic region between a pair of adjacent essential genes, and culturing the transformed host cell under conditions sufficient to express the GOI, wherein each of the adjacent essential genes of said pair has a transcription start site (TSS) and the distance between the TSSs is at least 80 nt and less than 20,000 nt.

12. A method of modifying a mammalian cell, the method comprising site-directed chromosomal integration of a heterologous gene of interest (GOI) within an intergenic region between a pair of adjacent essential genes, wherein each of the adjacent essential genes of said pair has a transcription start site (TSS) and the distance between the TSSs is at least 80 nt and less than 20,000 nt.

13. The method of claim 12, wherein the site-directed chromosomal integration is performed using any one of a programmable nuclease, a transcription activator-like effector nuclease (TALEN), a CRISPR endonuclease, or an Argonaute protein for chromosomal integration.

14-17. (canceled)

18. The method of claim 12, wherein the method further comprises differentiating an induced pluripotent stem (iPS) cell.

19. The method of claim 18, wherein the iPS cell is differentiated to generate a neuron or a cardiomyocyte.

20. The method of claim 18, wherein the iPS cell is differentiated to generate a neuron.

21. The method of claim 18, wherein the iPS cell is differentiated to generate a cardiomyocyte.

22. The method of claim 12, wherein the method comprises generating an iPS cell.

23. A method of treating a disease in a subject in need thereof, the method comprising administering the host cell of claim 1 to the subject.

24. The method of claim 23, wherein the host cell is an immune effector cell and the GOI encodes a chimeric antigen receptor (CAR).
